(19) United States
(12) Patent Application Publication
(10) Pub. No.: US 2012/0041061 A1
(43) Pub. Date: Feb. 16, 2012

(54) USE OF PRODRUGS OF GABA ANALOGS FOR TREATING DISEASES

(75) Inventors: Ronald W. Barrett, Saratoga, CA (US); Kenneth C. Cundy, Redwood City, CA (US)

(73) Assignee: XenoPort Inc.

(21) Appl. No.: 13/283,469

(22) Filed: Oct. 27, 2011

Related U.S. Application Data
(63) Continuation of application No. 12/001,067, filed on Dec. 6, 2007.
(60) Provisional application No. 60/873,561, filed on Dec. 8, 2006.

(51) Int. Cl. A61K 31/27; A61P 11/00; A61P 11/14; A61P 25/16; A61P 11/06

(52) U.S. Cl. 514/484

(57) ABSTRACT

Methods of using prodrugs of GABA analogs and pharmaceutical compositions thereof to treat migraine, fibromyalgia, amyotrophic lateral sclerosis, irritable bowel syndrome, social phobia, Parkinson's disease, asthma, cough, or chronic obstructive pulmonary disease, and pharmaceutical compositions of prodrugs of GABA analogs useful in treating migraine, fibromyalgia, amyotrophic lateral sclerosis, irritable bowel syndrome, social phobia, Parkinson's disease, asthma, cough, or chronic obstructive pulmonary disease are disclosed.

This application claims the benefit under 35 U.S.C. §119(e) of U.S. Provisional Application Ser. No. 60/873,561 filed Dec. 8, 2006, which is incorporated by reference herein in its entirety.

### FIELD

Methods and compositions disclosed herein relate to methods of using prodrugs of GABA analogs and pharmaceutical compositions thereof to treat migraine, fibromyalgia, amyotrophic lateral sclerosis, irritable bowel syndrome, social phobia, Parkinson's disease, asthma, cough, or chronic obstructive pulmonary disease in patients and to pharmaceutical compositions of prodrugs of GABA analogs useful in treating migraine, fibromyalgia, amyotrophic lateral sclerosis, irritable bowel syndrome, social phobia, Parkinson's disease, asthma, cough, or chronic obstructive pulmonary disease.

### BACKGROUND

Migraine, fibromyalgia, irritable bowel syndrome, cough, asthma, and social phobia, are estimated to affect between 5% and 20% of the population. While less prevalent, amyotrophic lateral sclerosis and Parkinson's disease are significant neurodegenerative diseases. Chronic obstructive pulmonary disease is a major and increasing global health problem and is expected to become the third most common cause of death and the fifth most common cause of disability in the world by 2020.

The γ-aminobutyric acid (γ-aminobutyric acid is abbreviated herein as GABA) analog, gabapentin (1), has been approved in the United States for the treatment of epileptic seizures, diabetic neuropathy, post-herpetic neuralgia, and restless legs syndrome (Backonja et al., JAMA 1998, 280, 1831-36; Rose et al., Anaesthesia 2002, 57, 451-62). Pregabalin (2), another GABA analog, has been approved in the United States for the treatment of post-herpetic neuralgia. Gabapentin and/or pregabalin have also been shown or are proposed to be effective in treating a number of other medical disorders (Magnus, Epilepsia 1999, 40, S66-72) including migraine (see, e.g., Chronicle and Mulleners, Anticonvulsant drugs for migraine prophylaxis, The Cochrane Database of Systemic Reviews 2004, Issue 3; Mathew et al., Headache 2001, 41(2), 119-128; Mathew et al., Cephalalgia 1996, 16, 367; Wessely et al., Cephalalgia, 1987, 7, 477-78; Mathew, Headache 2001, November-December (Suppl), S18-S24; Di Trapani et al., Clin Ter 2000, 151, 145-148; and Capuano et al., Clin Ter 2004, 155(2-3), 79-87); fibromyalgia (see e.g., Nampiaparampil and Schmerling, Am J Manage Care 2004, 10, 794-800; Crofford, Curr Rheumatol Rep, 2004, 6, 274-80; Zareba, Drugs Today, 2005, 41(8), 509-516; and Dooley et al., U.S. Application Publication No. 2004/0180959), amyotrophic lateral sclerosis (see, e.g., Taylor, Rev Neurol, 1997 153(Suppl 1), S39-45; and Cory, Ann Pharmacother 1995, 29(11), 1160-61), irritable bowel syndrome (see, e.g., Taylor, Rev Neurol, 1997 153(Suppl 1), S39-45; and Cory, Ann Pharmacother 1995, 29(11), 1160-61), social phobia (see e.g., Pande et al., J Clin Psychopharmacol 1999, 19, 341-348; and Pande et al., J Clin Psychopharmacol 2000, 20(5), 544-546; Pande et al., J Clin Psychopharmacol 2004, 24(2), 141-149; Selak, Curr Opin Investig Drugs 2001, 2(6), 828-834; Lauria-Horner and Pohl, Expert Opin Investig Drugs 2003, 12(4), 663-672; Kasper et al., Eur Neuropsychopharmacol 2002, 12 (Suppl), S341-S342; Rickels et al., 2002, Int J Neuropsychopharmacol 2002, 5, 14-15; and Smith et al., Eur Neuropsychopharmacol 2002, 12, S350), Parkinson's disease (see e.g., Olson et al., Am J. Med 1997, 102(1), 60-6; Faulkner et al., Ann Pharmacother 2003, 37(2), 282-286; Marjama-Lyons and Koller, Drugs Aging 2000, 16(4), 273-278; and Van Blercom et al., Clin Neuropharmacol 2004, 27(3), 124-128), cough (Lee and Woo, Ann Oto Rhinol Laryngol 2005, 114(4), 253-7; Magistro, International Publication. No. WO 00/67742; and Mintz and Lee, Am J Med 2006, 119, e13-e15), and pulmonary diseases such as asthma, bronchial conditions, and chronic obstructive pulmonary disease (Lomia, International Application No. WO 00/66096; Shrier and Taylor, EP 1192944; Magistro, WO 00/67742; and Bertrand et al., US 2004/0143014).

The broad pharmaceutical activities of GABA analogs such as gabapentin (1) and pregabalin (2):

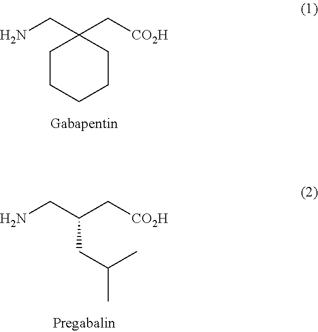

have stimulated intensive interest in preparing related compounds that have superior pharmaceutical properties in comparison to GABA, e.g., the ability to cross the blood-brain-barrier (see, Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, International Publication No. WO 92/09560; Silverman et al., International Publication No. WO 93/23383; Horwell et al., International Publication No. WO 97/29101, Horwell et al., International Publication No. WO 97/33858; Horwell et al., International Publication No. WO 97/33859; Bryans et al., International Publication No. WO 98/17627; Guglietta et al., International Publication No. WO 99/08671; Bryans et al., International Publication No. WO 99/21824; Bryans et al., International Publication No. WO 99/31057; Belliotti et al., International Publication No. WO 99/31074; Bryans et al., International Publication No. WO 99/31075; Bryans et al., International Publication No. WO 99/61424; Bryans et al., International Publication No. WO 2000/15611; Belliot et al., International Publication No. WO 00/31020; Bryans et al., International Publication No. WO 00/50027; and Bryans et al., International Publication No. WO 02/00209).

One significant problem associated with the clinical use of many GABA analogs, including gabapentin and pregabalin, is rapid systemic clearance. Consequently, these drugs require frequent dosing to maintain a therapeutic or prophylactic concentration in the systemic circulation (Bryans et al., Med. Res. Rev. 1999, 19, 149-177). For example, dosing regimens of 300-600 mg doses of gabapentin administered three times per day are typically used for anticonvulsive therapy. Higher doses (1800-3600 mg/day in three or four divided doses) are typically used for the treatment of neuropathic pain states. Doses of gabapentin up to 2400 mg/day with 300 mg administered eight times a day have been shown to be effective in treating migraine (see, e.g., Mathew et al., Headache 2001, 41, 119-128; Mathew, Cephalalgia 1996, 16, 367; Magnus-Miller et al., American Pain Society Program, 17th Annual Meeting, Abstract No. 645, San Diego, Calif., Nov. 5-8, 1998; Wessely et al., Cephalalgia 1987, 7(Suppl 6), 477-478; Di Trapani et al., Clin Ter 2000, 151, 145-148; and Capuano et al., Clin Ter 2004, 155(2-3), 79-87). Although oral sustained released formulations are conventionally used to reduce the dosing frequency of drugs that exhibit rapid systemic clearance, oral sustained release formulations of gabapentin and pregabalin have not been developed because these drugs are not absorbed via the large intestine. Rather, these compounds are typically absorbed in the small intestine by one or more amino acid transporters (e.g., the “large neutral amino acid transporter,” see Jezyk et al., Pharm. Res. 1999, 16, 519-526). The limited residence time of both conventional and sustained release oral dosage forms in the proximal absorptive region of the gastrointestinal tract necessitates frequent daily dosing of conventional oral dosage forms of these drugs, and has prevented the successful application of sustained release technologies to many GABA analogs.

One method for overcoming rapid systemic clearance of GABA analogs is to administer an extended release dosage formulation containing a colonically absorbed GABA analog prodrug (Gallop et al., U.S. Pat. Nos. 6,818,787, 6,972,341, 7,026,351, and 7,060,727; and U.S. Application Publication Nos. 2006/0122125 and 2005/0154057; and International Publication Nos. WO 02/100347 and WO 02/100349, each of which is incorporated by reference herein in its entirety). Sustained release formulations enable a colonically absorbed GABA analog prodrug to be absorbed over a wider region of the gastrointestinal tract than the parent drug including across the wall of the colon where sustained release oral dosage forms typically spend a significant portion of gastrointestinal transit time. These prodrugs are typically converted to the parent GABA analog upon absorption in vivo.

### SUMMARY

Therefore, there is a need for a method of treating migraine, fibromyalgia, amyotrophic lateral sclerosis, irritable bowel syndrome, social phobia, Parkinson's disease, asthma, cough, or chronic obstructive pulmonary disease, by delivering an agent, such as a prodrug of a GABA analog, for example, in a sustained release dosage form, with a reduced rate of systemic clearance, and without significant side effects.

In a first aspect, methods of treating a disease chosen from migraine, fibromyalgia, amyotrophic lateral sclerosis, irritable bowel syndrome, social phobia, Parkinson's disease, asthma, cough, or chronic obstructive pulmonary disease in a patient are provided comprising administering to a patient in need of such treatment a therapeutically effective amount of at least one compound chosen from Formula (I), Formula (II), Formula (III), and Formula (IV):

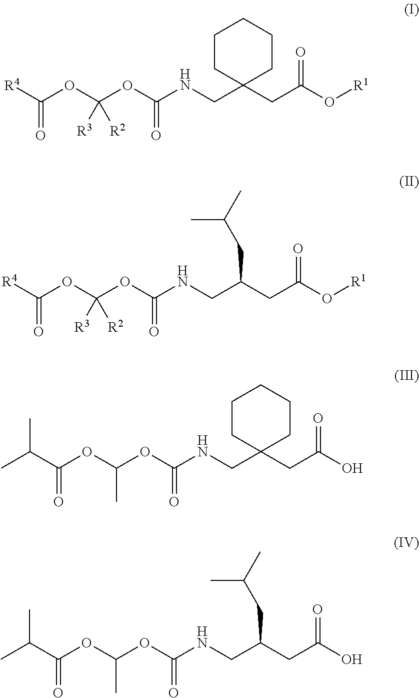

a pharmaceutically acceptable salt of any of the foregoing, a pharmaceutically acceptable solvate of any of the foregoing, and a pharmaceutically acceptable N-oxide of any of the foregoing, wherein:

R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are each independently chosen from hydrogen, alkyl,, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a ring chosen from a cycloalkyl, substituted cycloalkyl, cycloheteroalkyl, and substituted cycloheteroalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In a second aspect, methods of treating a disease chosen from migraine, fibromyalgia, amyotrophic lateral sclerosis, irritable bowel syndrome, social phobia, Parkinson's disease, asthma, cough, or chronic obstructive pulmonary disease in a patient are provided comprising administering to a patient in need of such treatment a pharmaceutical composition comprising a therapeutically effective amount of at least one compound chosen from Formula (I), Formula (II), Formula (III), and Formula (IV), a pharmaceutically acceptable salt of any of the foregoing, a pharmaceutically acceptable solvate of any of the foregoing, and a pharmaceutically acceptable N-oxide of any of the foregoing, together with a pharmaceutically acceptable vehicle.

Reference is now made in detail to embodiments provided by the present disclosure. The disclosed embodiments are not intended to be limiting of the claims.

### DETAILED DESCRIPTION

### Definitions

A dash (“-”) that is not between two letters or symbols is used to indicate a point of attachment for a moiety or substituent. For example, —CONH₂ is attached through the carbon atom.

“Alkyl” by itself or as part of another substituent refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl(allyl), prop-1-yn-1-yl, prop-2-yn-1-yl, etc.; butyls such as butan-1-yl, butan-2-yl, 2-methyl-propan-1-yl; 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group can have from 1 to 20 carbon atoms, in certain embodiments, from 1 to 10 carbon atoms, in certain embodiments, from 1 to 6 carbon atoms, and in certain embodiments, from 1 to 3 carbon atoms.

“Alkanyl” by itself or as part of another substituent refers to a saturated branched or straight-chain alkyl radical derived by the removal of one hydrogen atom from a single carbon atom of a parent alkane. Examples of alkanyl groups include, but are not limited to, methanyl; ethanyl; propanyls such as propan-1-yl and propan-2-yl (isopropyl), etc.; butanyls such as butan-1-yl, butan-2-yl(sec-butyl), 2-methyl-propan-1-yl(isobutyl), 2-methyl-propan-2-yl(t-butyl), etc.; and the like.

“Alkenyl” by itself or as part of another substituent refers to an unsaturated branched or straight-chain alkyl radical having at least one carbon-carbon double bond derived by the removal of one hydrogen atom from a single carbon atom of a parent alkene. The group may be in either the cis or trans conformation about the double bond(s). Examples of alkenyl groups include, but are not limited to, ethenyl; propenyls such as prop-1-en-1-yl, prop-1-en-2-yl, prop-2-en-1-yl(allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, etc.; and the like.

“Alkynyl” by itself or as part of another substituent refers to an unsaturated branched or straight-chain alkyl radical having at least one carbon-carbon triple bond derived by the removal of one hydrogen atom from a single carbon atom of a parent alkyne. Examples of alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is chosen from hydrogen, alkyl, cycloalkyl, cycloheteroalkyl, aryl, arylalkyl, heteroalkyl, heteroaryl, and heteroarylalkyl, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR³¹ where R³¹ is chosen from alkyl, cycloalkyl, cycloalkylalkyl, aryl, and arylalkyl, as defined herein. Examples of alkoxy groups include, but are not limited to, methoxy, ethoxy, propoxy, butoxy, cyclohexyloxy, and the like.

“Alkoxycarbonyl” by itself or as part of another substituent refers to a radical —C(O)OR³² where R³² represents an alkyl, as defined herein. Examples of alkoxycarbonyl groups include, but are not limited to, methoxycarbonyl, ethoxycarbonyl, propoxycarbonyl, and butoxycarbonyl, and the like.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 5 to 20 carbon atoms, and in certain embodiments, from 5 to 12 carbon atoms. Aryl, however, does not encompass or overlap in any way with heteroaryl, separately defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound or metabolite thereof in a biological fluid in a patient as a function of time following administration of the compound to the patient. In certain embodiments, the compound can be a prodrug and the metabolite can be a drug. Examples of biological fluids include plasma and blood. The AUC may be determined by measuring the concentration of a compound or metabolite thereof in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the present disclosure, an AUC for a GABA analog or metabolite thereof may be determined by measuring the concentration of the GABA analog or metabolite thereof in the plasma or blood of a patient following administration of a compound of Formula (I), Formula (II), Formula (III), or Formula (IV) to the patient.

“Carbamoyl” by itself or as part of another substituent refers to the radical —C(O)NR⁵⁰R⁵¹ where R⁵⁰ and R⁵¹ are independently chosen from hydrogen, alkyl, cycloalkyl, and aryl as defined herein.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to maximum concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the maximum concentration of a drug in the plasma or blood of a patient following administration of a dose of the drug or prodrug to the patient.

“Tₘₐₓ^{”} is the time to the maximum (peak) concentration (Cₘₐₓ) of a drug in the plasma or blood of a patient following administration of a dose of the drug or prodrug to the patient.

“Compounds” of Formula (I), Formula (II), Formula (III), and Formula (IV) disclosed herein, also referred to as “compounds provided by the present disclosure” include any specific compounds within these formulae. Compounds may be identified either by their chemical structure and/or chemical name. When the chemical structure and chemical name conflict, the chemical structure is determinative of the identity of the compound. The compounds described herein may comprise one or more stereogenic centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds of Formula (I), Formula (II), Formula (III), and Formula (IV) include, but are not limited to, optical isomers of compounds of Formula (I), Formula (II), Formula (III), and Formula (IV), racemates thereof, and other mixtures thereof. In such embodiments, the single enantiomers or diastereomers, i.e., optically active forms, can be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column. In addition, compounds of Formula (I), Formula (II), Formula (III), and Formula (IV) include Z- and E-forms (or cis- and trans-forms) of equilibrium forms with double bonds.

Compounds of Formula (I), Formula (II), Formula (III), and Formula (IV) may also exist in several tautomeric forms including an enol form, a keto form, and combinations thereof, where such forms are possible. Compounds of Formula (I) Formula (II), Formula (III), and Formula (IV) may also exist in an aza-enol form, an aza-keto form, and combinations thereof, wherein the Z-E tautomerism is with respect to the carbamate bond. Accordingly, the chemical structures depicted herein encompass all possible tautomeric forms of the illustrated compounds. Compounds of Formula (I), Formula (II), Formula (III), and Formula (IV) also include isotopically labeled compounds where one or more atoms have an atomic mass different from the atomic mass conventionally found in nature. Examples of isotopes that may be incorporated into the compounds disclosed herein include, but are not limited to, ²H, 3_{H}, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated, or N-oxides. Certain compounds may exist in multiple crystalline or amorphous forms. Compounds of Formula (I), Formula (II), Formula (III), and Formula (IV) include pharmaceutically acceptable salts of any of the foregoing, pharmaceutically acceptable solvates of any of the foregoing, as well as crystalline forms of any of the foregoing.

Further, when partial structures of the compounds are illustrated, an asterisk (*) indicates the point of attachment of the partial structure to the rest of the molecule.

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₅₋₁₂ cycloalkyl.

“Cycloheteroalkyl” by itself or as part of another substituent refers to a saturated or partially unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Examples of cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“GABA analog” refers to a compound having the following structure:

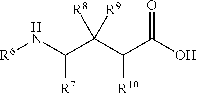

wherein:

R⁶ is hydrogen, or R⁶ and R¹⁰ together with the atoms to which they are bonded form a ring chosen from an azetidine, substituted azetidine, pyrrolidine, and substituted pyrrolidine ring;

R⁷ and R¹⁰ are independently chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl; and

R⁸ and R⁹ are independently chosen from hydrogen, alkyl, substituted alkyl, acyl, substituted acyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R⁸ and R⁹ together with the carbon atom to which they are bonded form a ring chosen from a cycloalkyl, substituted cycloalkyl, cycloheteroalkyl, substituted cycloheteroalkyl, and bridged cycloalkyl ring.

In certain embodiments of a GABA analog, each substituent is independently chosen from halogen, —NH₂, —OH, —CN, —COON, —C(O)NH₂, —C(O)OR⁵, and —NR⁵₃⁺ wherein each R⁵ is independently C₁₋₃ alkyl.

In certain embodiments of a GABA analog, R⁶ is hydrogen.

In certain embodiments of a GABA analog, R⁶ is hydrogen, R⁷ is hydrogen, R¹⁰ is hydrogen,,and R⁸ and R⁹ together with the carbon atom to which they are bonded form a cyclohexyl ring.

In certain embodiments of a GABA analog, R⁶ is hydrogen, R⁷ is hydrogen, R¹⁰ is hydrogen, R⁸ is hydrogen, and R⁹ is isobutyl.

In certain embodiments, a GABA analog is chosen from gabapentin and pregabalin.

“Halogen” refers to a fluoro, chloro, bromo, or iodo group.

“Heteroalkyl” by itself or as part of another substituent refer to an alkyl group in which one or more of the carbon atoms (and any associated hydrogen atoms) are independently replaced with the same or different heteroatomic groups. Examples of heteroatomic groups include, but are not limited to, —O—, —S—, —O—O—, —S—S—, —O—S—, —NR³⁷R³⁸—, ═N—N═, —N═N—, —N═N—R³⁹R⁴⁰, —PR⁴¹—, —P(O)₂—, —POR⁴²—, —O—P(O)₂—, —SO—, —SO₂—, —SnR⁴³R⁴⁴—, and the like, where R³⁷, R³⁸, R³⁹, R⁴⁰, R⁴¹, R⁴², R^{43,} and R⁴⁴ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, or substituted heteroarylalkyl. Where a specific level of saturation is intended, the nomenclature “heteroalkanyl,” “heteroalkenyl,” or “heteroalkynyl” is used.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 10-membered heteroaryl. In certain embodiments heteroaryl groups are those derived from thiophene, pyrrole, benzothiophene, benzofuran, indole, pyridine, quinoline, imidazole, oxazole, or pyrazine

“Heteroarylalkyl” by itself or as part of another substituent refers to an acyclic alkyl radical in which one of the hydrogen atoms bonded to a carbon atom, is replaced with a heteroaryl group. Typically a terminal or sp³ carbon atom is the atom replaced with the heteroaryl group. Where specific alkyl moieties are intended, the nomenclature “heteroarylalkanyl,” “heteroarylalkenyl,” and “heterorylalkynyl” is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Migraine” means a symptom complex occurring periodically that is characterized by one or more of the following symptoms: pain in the head that may be exacerbated by movement or physical activity, nausea and/or vomiting, diarrhea, photophobia, visual disturbances, including scintillating appearances of light, alternations in consciousness including seizure, syncope, and confused state, vertigo, light-headedness, scalp tenderness, or paresthesia. The particular combination of symptoms and their frequency and severity are used to classify migraine into numerous subclasses (see, e.g., Headache Classification Committee of the International Headache Society: Yje, International Classification of Headache Disorders, 2^{nd} edition, Cephalalagia 2004, 24 (suppl. 1), Blackwell Publishing). Not every migraine needs to meet all migraine criteria to be classified as migraine. For example, a person may have a left-temporal throbbing headache of moderate intensity worsened by physical activity. These headache features meet migraine criteria. However, this headache may not be accompanied by nausea or hypersensitivity to light or noise and therefore not fulfill all the criteria for migraine. Furthermore, if some of this person's other headaches meet all the migraine criteria, then the headache can also be classified as a migraine.

“Fibromyalgia” means a symptom complex occurring periodically that is characterized by aching and pain in the muscles, tendons, and joints all over the body, but especially along the spine. The body also is tender to touch in specific areas called tender or trigger points. Other symptoms associated with fibromyalgia pain include sleep disturbance, depression, daytime tiredness, headaches, alternating diarrhea and constipation, numbness and tingling in the hands and feet, feelings of weakness, memory difficulties, and dizziness. The etiology of fibromyalgia is unknown. The American College of Rheumatology's classification criteria for fibromyalgia include diffuse soft tissue pain of at least 3 months’ duration and pain on palpation in at least 11 of 18 paired tender points (see, e.g., Nampiaparampil, et al., Am J Manag Care 2004, 10, 794-800; Kranzler et al., U.S. Application Publication No. 2003/0130353; Taylor et al., U.S. Application Publication No. 2004/0138305; Dooley et al., U.S. Application Publication No. 2004/0180959; and Zeldis et al., U.S. Application Publication No. 2005/0119194). Fibromyalgia can be classified by the combination of symptoms and by the severity and frequency of the symptoms (see e.g., Nampiaparampil, et al., Am J Manag Care 2004, 10, 794-800). For example, a person may have pain in 8 to 10 tender points, which alone may not meet the criteria for fibromyalgia, but if accompanied by other symptoms such as morning stiffness, fatigue, and sleep disturbance these features fulfill the criteria for fibromyalgia.

“Amyotrophic lateral sclerosis” (ALS) is a chronic, progressive, almost invariably fatal neurological disease. ALS is marked by gradual degeneration of the nerve cells in the central nervous system that control voluntary muscle movement. ALS symptoms typically begin in the limbs with patients experiencing awkwardness when walking or running or difficulty with simple tasks requiring manual dexterity, or can experience difficulty in speaking clearly. Regardless of the part of the body first affected by ALS, muscle weakness and atrophy spread to other parts of the body as the disease progresses. ALS patients may have increasing problems with moving, swallowing (dysphagia), speaking or forming words (dysarthria), tight and stiff muscles (spasticity), exaggerated reflexes (hyperreflexia) including an overactive gag reflex, Babinski's sign in which the large toe extends upward as the sole of the foot is stimulated in a certain way, muscle weakness and atrophy, muscle cramps, muscle twitches (fasciculations), and/or the pseudobulbar affect in which a patient uncontrollable laughs or cries. Because ALS affects only motor neurons, the disease does not impair a person's mind, personality, intelligence, memory or a person's ability to see, smell, taste, hear, feel touch, or control eye muscles and bladder and bowel function. Weakening of the diaphragm and intercostal muscles typically lead to respiratory failure or pneumonia.

ALS diagnosis requires that patients have sign and symptoms of both upper and lower motor neuron damage that cannot be attributed to other causes. Tests used to diagnose ALS include electromyography, nerve conduction velocity, and magnetic resonance image. Biomarkers for ALS have also been identified (see e.g., Denger et al., Neurodegener Dis 2005, 2(3-4), 177-84; and Bowser et al., Expert Rev Mol Diagn 2006 May, 6(3), 387-98). ALS includes all classifications of ALS known in the art, including, classical ALS typically affecting both lower and upper motor neurons; primary lateral sclerosis typically affecting only the upper motor neurons; progressive bulbar palsy typically beginning with difficulties swallowing, chewing, or speaking; progressive muscular atropy typically affecting only the lower motor neurons; and familial ALS, which is a genetic version of ALS.

“Irritable bowel syndrome” is a functional bowel disorder characterized by abdominal pain and changes in bowel habits, which are not associated with any abnormalities seen on routine clinical testing. Typical symptoms include lower abdominal pain, and bloating associated with alternation of bowel habits and abdominal discomfort relieved with defecation. Intestinal bowel disorder (IBS) can be associated with stress, chronic pelvic pain, fibromyalgia, chronic fatigue syndrome, headache, sexual dysfunction, sleep disturbances, and certain mental disorders.

IBS may be diagnosed using Rome II Diagnostic criteria (Thompson et al., Gut. 1999, 45(suppl 2), 1143-47; and Rome 11: Functional Gastrointestinal Disorders, Diagnosis, Pathophysiology and Treatment. A Multinational Consensus, Drossman et al., Eds, Allen Press, Lawrence K S, 2000), generally summarized as abdominal discomfort for 12 weeks or more in the preceding 12 months accompanied by 2 or more of the following: relief of abdominal discomfort with defecation; onset associated with a change in stool frequency; and onset associated with a change in stool form. IBS can be classified as diarrhea-predominant, constipation-predominant, IBS with alternating stool pattern, and post-infectious IBS. IBS patients also report non-gastrointestinal symptoms such as fatigue, muscle pain, sleep disturbances, and sexual dysfunction, low back pain, and headache.

“Social phobia” or “social anxiety disorder” is a psychiatric anxiety disorder characterized by significant anxiety induced by exposure to certain social or performance situations, often resulting in avoidance. Social phobia includes diseases and conditions classified under DSM-IV 300.23 (Diagnostic and Statistical manual of Mental Disorders, DSM-IV-TR, 4^{th} Ed., Am. Psychiatric Assoc., pages 450-456, 2000). Features commonly associated with social phobia include hypersensitivity to criticism, negative evaluation, or rejection; difficulty being assertive; and low self-esteem or feeling of inferiority. Social phobia may be associated with other anxiety disorders, mood disorders, substance-related disorders, and bulimia nervosa. Social phobia can include other avoidant personality disorders such as global social phobia, specific social phobia, simple phobia, agoraphobia, apiphobia, tropophobia, astrapophobia, triskaidekaphobia, blennophobia, thalassophobia, claustrophobia, spheksophobia, cynophobia, sciophobia, decidophobia, eletrophobia, scholionophobia, eremophobia, pyrophobia, gamophobia, pnigerophobia, ophidiophobia, odynophobia, nyctophobia, ochlophobia, musophobia, keraunophobia, katagelophobia, kakorraphiophobia, hydrophobia, gynophobia, gatophobia, gephyrophobia, acrophobia, and amathophobia.

“Parkinson's disease” is a clinical syndrome comprising bradykinesia (slowness and poverty of movement), muscular rigidity, resting tremor (which usually abates during voluntary movement), and an impairment of postural balance leading to disturbance of gait and falling. Other symptoms include gait and posture disturbances such as shuffling, decreased arm swing, turning “en bloc,” stooped, forward-reflexed posture, festination, gait freezing and dystonia; speech and swallowing disturbances such as hypophonia, festinating speech, drooling, non-motor causes of speech/language disturbance in both expressive and receptive language, and dysphagia; as well as fatigue, masked facies, micropgraphia, impaired fine motor dexterity and coordination, impaired gross motor coordination, and poverty of movement. Non-motor mood disturbances associated with Parkinson's disease include mood disturbances such as depression; cognitive disturbances such as slowed reaction time, executive dysfunction, dementia, memory loss, and medication effects; sleep disturbances such as excessive daytime somnolence, insomnia, and disturbances in REM sleep; sensation disturbances such as impair visual perception, dizziness and fainting, impaired proprioception, reduction or loss of sense of smell, and pain; and autonomic disturbances such as oily skin and seborrheic dermatitis, urinary incontinence, constipation and gastric dysmotility, altered sexual function, and weight loss.

The Unified Parkinson's disease Rating scale is the primary clinical tool used for the diagnosis of Parkinson's disease (see e.g., Gelb et al., Arch Neurol 1999, 56(1), 33-9; and Goetz, Mov Disord 2003 July, 18(7), 738-50).

Cough includes acute and chronic cough of any type, etiology, or pathogenesis, and in particular cough associated with laryngeal sensory neuropathy.

Asthma is an acute or chronic disorder characterized by widespread and largely reversible reduction in the caliber of bronchi and bronchioles, due in varying degrees to smooth muscle spasm, mucosal edema, and excessive mucus in the lumens of the airway. Asthma includes atopic asthma, non-atomic asthma, allergic asthma, atopic bronchial IgE mediated asthma, bronchial asthma, essential asthma, true asthma, intrinsic asthma caused by pathophysiologic disturbances, extrinsic asthma caused by environmental factors, essential asthma of unknown or unapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome.

Chronic obstructive pulmonary disease includes chronic or acute chronic or acute bronchoconstriction, large airway obstruction, chronic bronchitis, small airways obstruction, and emphysema; pneuomconiosis of whatever type, etiology, or pathogenesis, such as aluminosis, anthracosis, asbestosis, chalicosis, ptilosis, siderosis, silicosis, byssinosis, and talc pneumoconiosis; bronchitis of whatever type, etiology, or pathogenesis, such as acute bronchitis, acute laryngotracheal bronchitis, arachidic bronchitis, catarrhal bronchitis, cropus bronchitis, dry bronchitis, infectious asthmatic bronchitis, productive bronchitis, staphylococcus or streptococcal bronchitis, and vesicular bronchitis; and bronchiectasis of whatever type, etiology, or pathogenesis, such as cylindric bronchiectasis, sacculated bronchiectasis, fusiform bronchiectasis, capillary bronchiectasis, cystic bronchiectasis, dry bronchiectasis, and follicular bronchiectasis.

“N-oxide” refers to the zwitterionic nitrogen oxide of a tertiary amine base.

“Parent aromatic ring system” refers to an unsaturated cyclic or polycyclic ring system having a conjugated 90 (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, beniene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom. Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, for example, a human.

“Pharmaceutically acceptable” refers to approved or approvable by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound, which possesses the desired pharmacological activity of the parent compound and which is pharmaceutically acceptable. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure can be administered to a patient and which does not destroy the pharmacological activity thereof and which is non-toxic when administered in doses sufficient to provide a therapeutically effective amount of the compound.

“Pharmaceutical composition” refers to at least one compound of Formula (I), Formula (II), Formula (III), or Formula (IV) and at least one pharmaceutically acceptable vehicle, with which the at least one compound of Formula (I), Formula (II), Formula (III), or Formula (IV) is administered to a patient.

“Prodrug” refers to a derivative of a drug molecule that requires a transformation within the body to release the active drug. Prodrugs are frequently, although not necessarily, pharmacologically inactive until converted to the parent drug. Compounds of Formula (I), Formula (II), Formula (III), and Formula (IV) are prodrugs of GABA analogs that can be metabolized within a patient's body to form the corresponding GABA analog parent drug.Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach, or the agent may be supplied exogenously. For example, for a prodrug of Formula (I) and Formula (II), the drug is gabapentin or pregabalin, respectively, and the promoiety has the structure:

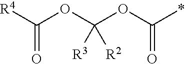

and, for a prodrug of Formula (III) and Formula (IV), the drug is gabapentin or pregabalin, respectively, and the promoiety has the structure:

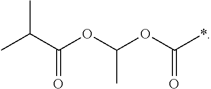

“Protecting group” refers to a grouping of atoms, which when attached to a reactive group in a molecule masks, reduces, or prevents that reactivity. Examples of protecting groups can be found in Wuts and Greene, “Protective Groups in Organic Synthesis,” John Wiley & Sons, 4th ed. 2006; Harrison et al., “Compendium of Organic Synthetic Methods,” Vols. 1-11, John Wiley & Sons 1971-2003; Larock “Comprehensive Organic Transformations,” John Wiley & Sons, 2nd ed. 2000; and Paquette, “Encyclopedia of Reagents for Organic Synthesis,” John Wiley & Sons, 11th ed. 2003. Examples of amino protecting groups include, but are not limited to, formyl, acetyl, trifluoroacetyl, benzyl, benzyloxycarbonyl (CBZ), tert-butoxycarbonyl (Boc), trimethylsilyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like. Examples of hydroxy protecting groups include, but are not limited to, those in which the hydroxy group is either acylated or alkylated such as benzyl, and trityl ethers as well as alkyl ethers, tetrahydropyranyl ethers, trialkylsilyl ethers, and allyl ethers.

“Solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules are water.

“Substituted” refers to a group in which one or more hydrogen atoms are independently replaced with the same or different substituent(s). Examples of substituents include, but are not limited to, -M, —R⁶⁰, —O⁻, ═O, —OR⁶⁰, —SR⁶⁰, —S⁻, ═S, —NR⁶⁰R⁶¹, ═NR⁶⁰, CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, and —C(NR⁶²)NR⁶⁰R⁶¹ where M is independently a halogen; R⁶⁰, R⁶¹, R⁶², and R⁶³ are independently chosen from hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl, and substituted heteroaryl, or R⁶⁰ and R⁶¹ together with the nitrogen atom to which they are bonded form a ring chosen from a cycloheteroalkyl and substituted cycloheteroalkyl ring.

In certain embodiments, each substituent group is independently chosen from halogen, —NH₂, —OH, —CN, —CF₃, —COOH, —C(O)NH₂, —C(O)OR⁶⁴, and —NR⁶⁴₂, wherein each R⁶⁴ is independently C₁₋₃ alkyl.

“Sustained release” refers to release of a compound from a pharmaceutical composition dosage form at a rate effective to achieve a therapeutic or prophylactic concentration of the compound or active metabolite thereof, in the systemic circulation of a patient over a prolonged period of time relative to that achieved by administration of an immediate release formulation of the same compound by the same route of administration. In some embodiments, release of a compound occurs over a time period of at least about 4 hours, such as at least about 8 hours, at least about 12 hours, at least about 16 hours, at least bout 20 hours, and in some embodiments, at least about 24 hours.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

In certain embodiments, the terms “treating” and “treatment” and “to treat” refer to preventing, reducing, or eliminating migraine, fibromyalgia, amyotrophic lateral sclerosis, irritable bowel syndrome, social phobia, Parkinson's disease, asthma, cough, or chronic obstructive pulmonary disease and/or the accompanying symptoms of migraine, fibromyalgia, amyotrophic lateral sclerosis, irritable bowel syndrome, social phobia, Parkinson's disease, asthma, cough, or chronic obstructive pulmonary disease in a patient. Treatment refers to any indicia of success in prevention, reduction, or elimination or amelioration of migraine, fibromyalgia, amyotrophic lateral sclerosis, irritable bowel syndrome, social phobia, Parkinson's disease, asthma, cough, or chronic obstructive pulmonary disease including any objective or subjective parameter such as abatement, remission, diminishing of symptoms, prevention, or lessening of migraine, fibromyalgia, amyotrophic lateral sclerosis, irritable bowel syndrome, social phobia, Parkinson's disease, asthma, cough, or chronic obstructive pulmonary disease symptoms or making the condition more tolerable to the patient, making the migraine, fibromyalgia, amyotrophic lateral sclerosis, irritable bowel syndrome, social phobia, Parkinson's disease, asthma, cough, or chronic obstructive pulmonary disease less debilitating, or improving a patient's physical or mental well-being. For example, success of treatment by methods of treating migraine; fibromyalgia, amyotrophic lateral sclerosis, irritable bowel syndrome, social phobia, Parkinson's disease, asthma, cough, or chronic obstructive pulmonary disease provided by the present disclosure may be measured by comparing the frequency and severity of migraine attacks in the year before treatment with a GABA analog prodrug provided by the present disclosure was initiated, with the frequency and/or severity of migraine, fibromyalgia, amyotrophic lateral sclerosis, irritable bowel syndrome, social phobia, Parkinson's disease, asthma, cough, or chronic obstructive pulmonary disease during the year following the initiation of treatment. The prevention, treatment, or amelioration of migraine, fibromyalgia, amyotrophic lateral sclerosis, irritable bowel syndrome, social phobia, Parkinson's disease, asthma, cough, or chronic obstructive pulmonary disease symptoms may be based on objective or subjective parameters, including the results of a physical examination, or personal interview regarding symptom severity and quality of life, or any other appropriate means known in the art.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The “therapeutically effective amount” may vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

### GABA Analog Prodrugs

In certain embodiments, a prodrug of a GABA analog is chosen from at least one compound of Formula (I), Formula (II), Formula (III), and Formula (IV):

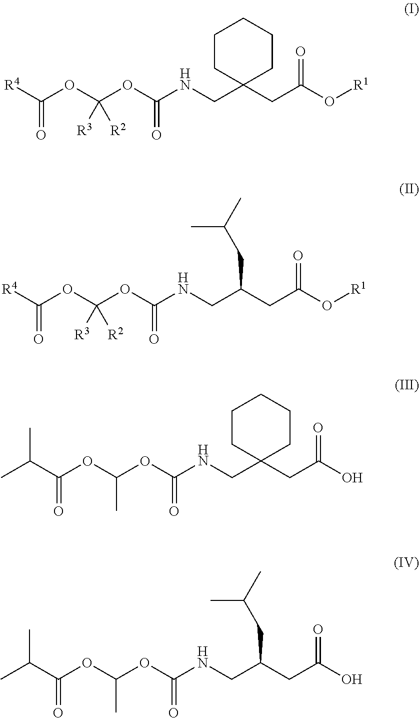

a pharmaceutically acceptable salt of any of the foregoing, a pharmaceutically acceptable solvate of any of the foregoing, and a pharmaceutically acceptable N-oxide of any of the foregoing, wherein:

R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a ring chosen from a cycloalkyl, substituted cycloalkyl, cycloheteroalkyl, and substituted cycloheteroalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In certain embodiments, for example, wherein R⁴ is substituted alkyl, each substituent group is independently chosen from halogen, —NH₂, —OH, —CN, —CF₃, —COOH, —C(O)NH₂, —C(O)OR⁵, and —NR⁵₂ wherein each R⁵ is independently C₁₋₃ alkyl.

In certain embodiments of compounds of Formula (I) and (II), R¹ is hydrogen.

In certain embodiments of compounds of Formula (I) and (II), R² and R³ are independently chosen from hydrogen and C₁₋₆ alkyl.

In certain embodiments of compounds of Formula (I) and (II), at least one of R² and R³ is other than hydrogen.

In certain embodiments of compounds of Formula (I) and (II), R³ is chosen from methyl, ethyl, n-propyl, isopropyl, n-butyl, isobutyl, and sec-butyl, and R² is hydrogen.

In certain embodiments of compounds of Formula (I) and (II), R³ is chosen from methyl, ethyl, n-propyl, and isopropyl. In certain embodiments of compounds of Formula (I) and (II), R² is hydrogen, and R³ is chosen from methyl, ethyl, n-propyl, and isopropyl.

In certain embodiments of compounds of Formula (I) and (II), R⁴ is chosen from C₁₋₆ alkyl and C₁₋₆ substituted alkyl. In certain embodiments of compounds of Formula (I) and (II) wherein R⁴ is chosen from C₁₋₆ substituted alkyl, the substituent group is chosen from halogen, —NH₂, —OH, —CN, —CF₃, —COOH, —C(O)NH₂, —C(O)OR⁵, and —NR⁵₂ wherein each R⁵ is independently C₁₋₃ alkyl.

In certain embodiments of compounds of Formula (I) and (II), R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formula (I) and (II), R⁴ is chosen from methyl, ethyl, n-propyl, isopropyl, n-butyl, and isobutyl.

In certain embodiments of compounds of Formula (I) and (II), R¹ and R² are each hydrogen, R³ is C₁₋₆ alkyl, and R⁴ is chosen from C₁₋₆ alkyl and C₁₋₆ substituted alkyl. In certain embodiments of compounds of Formula (I) and (II), wherein R¹ and R² are each hydrogen, R³ is C₁₋₆ alkyl, and R⁴ is chosen from C₁₋₆ substituted alkyl, each substituent group is independently chosen from halogen, —NH₂, —OH, —CN, —CF₃, —COOH, —C(O)NH₂, —C(O)OR⁵, and —NR⁵₂ wherein each R⁵ is independently C₁₋₃ alkyl.

In certain embodiments of compounds of Formula (I) and (II), R¹ and R² are each hydrogen, R³ is chosen from methyl, ethyl, n-propyl, isopropyl, n-butyl, isobutyl, and sec-butyl, and R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formula (I) and (II), R¹ and R² are each hydrogen, R³ is chosen from methyl, ethyl, n-propyl, and isopropyl, and R⁴ is chosen from methyl, ethyl, n-propyl, isopropyl, n-butyl, and isobutyl.

In certain embodiments, the compound of Formula (I) wherein R⁴ is isopropyl, R² is hydrogen, and R³ is methyl, is 1-{[(α-isobutanoyloxyethoxy)carbonyl]aminomethyl}-1-cyclohexane acetic acid (compound of Formula (III)), a pharmaceutically acceptable salt thereof, a pharmaceutically acceptable solvate of any of the foregoing, or a pharmaceutically acceptable N-oxide of any of the foregoing.

In certain embodiments, the compound of Formula (I) wherein R⁴ is isopropyl, R² is hydrogen, and R³ is methyl, is a crystalline form of 1-{[(α-isobutanoyloxyethoxy)carbonyl]aminomethyl}-1-cyclohexane acetic acid (compound of Formula (III)) as disclosed in Estrada et al., U.S. Application Publication No. 2005/015405, which is incorporated by reference herein in its entirety. In certain embodiments, crystalline 1-{[(α-isobutanoyloxyethoxy)carbonyl]aminomethyl}-1-cyclohexane acetic acid has characteristic absorption peaks at 7.0°±0.3°, 8.2°±0.3°, 10.5°±0.3°, 12.8°±0.3°, 14.9°±0.3°, 16.4°±0.3°, 18.1°±0.3°, 18.9°±0.3°, 20.9°±0.3°, 23.3°±0.3°, 25.3°±0.3°, and 26.6°±0.3° in an X-ray powder diffractogram. In certain embodiments, crystalline 1-{[(α-isobutanoyloxyethoxy)carbonyl]aminomethyl}-1-cyclohexane acetic acid has a melting point range from about 63° C. to about 64° C., in certain embodiments, from about 64° C. to about 66° C., and in certain embodiments, from about 63° C. to about 66° C.

Examples of compounds of Formula (I) include: 1-{[(α-Acetoxyethoxy)carbonyl]aminomethyl}-1-cyclohexane acetic acid, 1-{[(α-Propanoyloxyethoxy)carbonyl]aminomethyl}-1-cyclohexane acetic acid, 1-{[(α-Butanoyloxyethoxy)carbonyl]aminomethyl}-1-cyclohexane acetic acid, 1-{[(α-Isobutanoyloxyethoxy)carbonyl]aminomethyl}-1-cyclohexane acetic acid (compound of Formula (III)), 1-{[(α-Pivaloxyethoxy)carbonyl]aminomethyl}-1-cyclohexane acetic acid, 1-{[α-Acetoxymethoxy)carbonyl]aminomethyl}-1-cyclohexane acetic acid, 1-{[(α-Propanoyloxymethoxy)carbonyl]aminomethyl}-1-cyclohexane acetic acid, 1-{[(α-Butanoyloxymethoxy)carbonyl]aminomethyl}-1-cyclohexane acetic acid, 1-{[(α-Isobutanoyloxymethoxy)carbonyl]aminomethyl}-1-cyclohexane acetic acid, 1-{[(α-Pivaloxymethoxy)carbonyl]aminomethyl1-1-cyclohexane acetic acid, 1-{[(α-Acetoxypropoxy)carbonyl]aminomethyl}-1-cyclohexane acetic acid, 1-{[(α-Propanoyloxypropoxy)carbonyl]aminomethyl}-1-cyclohexane acetic acid, 1-{[(α-Butanoyloxypropoxy)carbonyl]aminomethyl}-1-cyclohexane acetic acid, 1-{[(α-Isobutanoyloxypropoxy)carbonyl]aminomethyl}-1-cyclohexane acetic acid, 1-{[(α-Pivaloxypropoxy)carbonyl]aminomethyl}-1-cyclohexane acetic acid, 1-{[(α-Acetoxyisopropoxy)carbonyl]aminomethyl}-1-cyclohexane acetic acid, 1-{[(α-Propanoyloxyisopropoxy)carbonyl]aminomethyl}-1-cyclohexane acetic acid, 1-{[(α-Butanoyloxyisopropoxy)carbonyl]aminomethyl}-1-cyclohexane acetic acid, 1-{[(α-Isobutanoyloxyisopropoxy)carbonyl]aminomethyl}-1-cyclohexane acetic acid, 1-{[(α-Pivaloxyisopropoxy)carbonyl]aminomethyl}-1-cyclohexane acetic acid, 1-{[(α-Acetoxybutoxy)carbonyl]aminomethyl}-1-cyclohexane acetic acid, 1-{[(α-Propanoyloxybutoxy)carbonyl]aminomethyl}-1-cyclohexane acetic acid, 1-{[(α-Butanoyloxybutoxy)carbonyl]aminomethyl}-1-cyclohexane acetic acid, 1-{[(α-Isobutanoyloxybutoxy)carbonyl]aminomethyl}-1-cyclohexane acetic acid, 1-{[(α-Pivaloxybutoxy)carbonyl]aminomethyl}-1-cyclohexane acetic acid, pharmaceutically acceptable salts of any of the foregoing, pharmaceutically acceptable solvates of any of the foregoing, and pharmaceutically acceptable N-oxides of any of the foregoing.

Examples of compounds of Formula (II) include: 3-{[(α-Acetoxyethoxy)carbonyl]aminomethyl}-5-methyl hexanoic acid, 3-{[(α-Propanoyloxyethoxy)carbonyl]aminomethyl}-5-methyl hexanoic acid, 3-{[(α-Butanoyloxyethoxy)carbonyl]aminomethyl}-5-methyl hexanoic acid, 3-{[(α-Isobutanoyloxyethoxy)carbonyl]aminomethyl}-5-methyl hexanoic acid (compound of Formula (IV)), 3-{[(α-Pivaloxyethoxy)carbonyl]aminomethyl}-5-methyl hexanoic acid, 3-{[(α-Acetoxymethoxy)carbonyl]aminomethyl}-5-methyl hexanoic acid, 3-{[(α-Propanoyloxymethoxy)carbonyl]aminomethyl}-5-methyl hexanoic acid, 3-{[(α-Butanoyloxymethoxy)carbonyl]aminomethyl}-5-methyl hexanoic acid, 3-{[(α-Isobutanoyloxymethoxy)carbonyl]aminomethyl}-5-methyl hexanoic acid, 3-{[(α-Pivaloxymethoxy)carbonyl]aminomethyl}-5-methyl hexanoic acid, 3-{[(α-Acetoxypropoxy)carbonyl]aminomethyl}-5-methyl hexanoic acid, 3-{[(α-Propanoyloxypropoxy)carbonyl]aminomethyl}-5-methyl hexanoic acid, 3-{[(α-Butanoyloxypropoxy)carbonyl]aminomethyl}-5-methyl hexanoic acid, 3-{[(α-Isobutanoyloxypropoxy)carbonyl]aminomethyl}-5-methyl hexanoic acid, 3-{[(α-Pivaloxypropoxy)carbonyl]aminomethyl}-5-methyl hexanoic acid, 3-{[(α-Acetoxyisopropoxy)carbonyl]aminomethyl}-5-methyl hexanoic acid, 3-{[(α-Propanoyloxyisopropoxy)carbonyl]aminomethyl}-5-methyl hexanoic acid, 3-{[(α-Butanoyloxyisopropoxy)carbonyl]aminomethyl}-5-methyl hexanoic acid, 3-{[(α-Isobutanoyloxyisopropoxy)carbonyl]aminomethyl}-5-methyl hexanoic acid, 3-{[(α-Pivaloxyisopropoxy)carbonyl]aminomethyl}-5-methyl hexanoic acid, 3-{[(α-Acetoxybutoxy)carbonyl]aminomethyl}-5-methyl hexanoic acid, 3-{[(α-Propanoyloxybutoxy)carbonyl]aminomethyl}-5-methyl hexanoic acid, 3-{[(α-Butanoyloxybutoxy)carbonyl]aminomethyl}-5-methyl hexanoic acid, 3-{[(α-Isobutanoyloxybutoxy)carbonyl]aminomethyl}-5-methyl hexanoic acid, 3{[(α-Pivaloxybutoxy)carbonyl]aminomethyl}-5-methyl hexanoic acid, pharmaceutically acceptable salts of any of the foregoing, pharmaceutically acceptable solvates of any of the foregoing, and pharmaceutically acceptable N-oxides of any of the foregoing.

In certain embodiments, the compound of Formula (II) is 3-{[(α-isobutanoyloxyethoxy)carbonyl]aminomethyl}-5-methyl hexanoic acid (compound of Formula (IV)), a pharmaceutically acceptable salt thereof, a pharmaceutically acceptable solvate of any of the foregoing, or a pharmaceutically acceptable N-oxide of any of the foregoing.

In certain embodiments, the compound of Formula (III) is 1-{[(α-isobutanoyloxyethoxy)carbonyl]aminomethyl}-1-cyclohexane acetic acid, a pharmaceutically acceptable salt thereof, a pharmaceutically acceptable solvate of any of the foregoing, or a pharmaceutically acceptable N-oxide of any of the foregoing.

In certain embodiments, the compound of Formula (IV) is 3-{[(α-isobutanoyloxyethoxy)carbonyl]aminomethyl}-5-methyl hexanoic acid, a pharmaceutically acceptable salt thereof, a pharmaceutically acceptable solvate of any of the foregoing, or a pharmaceutically acceptable N-oxide of any of the foregoing.

### Methods of Synthesizing Prodrugs of GABA Analogs

Methods of synthesizing prodrugs of GABA analogs, including methods of synthesizing compounds of structural Formula (I) Formula (II), Formula (III), and (IV) are disclosed in Gallop et al., PCT International Publication No. WO 02/100347, Gallop et al., U.S. Application Publication No. 2004/0077553, and Bhat et al., U.S. Application Publication No. 2006/0070715, each of which is incorporated by reference herein in its entirety. Other methods for synthesizing prodrugs of GABA analogs have also been disclosed (see Bryans et al., PCT International Publication No. WO 01/90052; U.K. Application GB 2,362,646; European Applications EP 1,201,240 and 1,178,034; Yatvin et al., U.S. Pat. No. 6,024,977; Gallop et al., PCT International Publication No. WO 02/28881; Gallop et al., PCT International Publication No. WO 02/28883; Gallop et al., International Publication No. WO 02/28411; Gallop et al., PCT International Publication No. WO 02/32376; and Gallop et al., PCT International Publication No. WO 02/42414).

### Methods of Use

In certain embodiments, a prodrug of a GABA analog or pharmaceutical composition thereof may be administered to a patient suffering from migraine, fibromyalgia, amyotrophic lateral sclerosis, irritable bowel syndrome, social phobia, Parkinson's disease, asthma, cough, or chronic obstructive pulmonary disease. The suitability of GABA analog prodrugs or pharmaceutical compositions thereof to treat migraine, fibromyalgia, amyotrophic lateral sclerosis, irritable bowel syndrome, social phobia, Parkinson's disease, asthma, cough, or chronic obstructive pulmonary disease may be determined by methods known to those skilled in the art.

Other methods, directed to new therapeutic uses of prodrugs of GABA analogs, including methods of using compounds of structural Formula (I), Formula (II), Formula (III), and Formula (IV) are disclosed in Barrett, U.S. Application Publication Nos. 2005/0090550, 2004/0254246, and 2005/0192353; and Tran, U.S. Application Publication Nos. 2007/0049626 and 2007/0049627, each of which is incorporated by reference herein in its entirety.

When used in the present methods of treatment, upon releasing a prodrug of a GABA analog in vivo, a dosage form comprising a GABA analog or pharmaceutical composition thereof may provide the GABA analog (e.g., gabapentin or pregabalin) in the systemic circulation of a patient. The promoiety or promoieties of the prodrug may be cleaved either chemically and/or enzymatically. One or more enzymes present in the intestinal lumen, intestinal tissue, blood, liver, brain, or any other suitable tissue of a mammal may cleave the promoiety or promoieties of the prodrug. The mechanism of cleavage is not important to the current methods. In certain embodiments, a GABA analog that is formed by cleavage of the promoiety or promoieties from the corresponding GABA analog prodrug does not contain substantial quantities of lactam contaminant (such as, less than about 0.5% by weight, for example, less than about 0.2% by weight, and in certain embodiments, less than about 0.1% by weight) for the reasons described in Augart et al., U.S. Pat. No. 6,054,482. The extent of release of lactam contaminant from a GABA analog prodrug may be assessed using standard in vitro analytical methods.

Some therapeutically effective GABA analogs, e.g., gabapentin and pregabalin, have poor passive permeability across the gastrointestinal mucosa, possibly because of their zwitterionic character at physiological pH. Gabapentin, pregabalin, and other GABA analogs are actively transported across the gastrointestinal tract by one or more amino acid transporters (e.g., the “large neutral amino acid transporter”). However, the large neutral amino acid transporter is expressed predominantly within cells lining the lumen of a limited region of the small intestine, which provides a limited window for drug absorption and leads to an overall dose-dependent drug bioavailability that decreases with increasing dose.

The compounds disclosed herein, for example the gabapentin prodrug 1-{[(α-isobutanoyloxyethoxy)carbonyl]aminomethyl}-1-cyclohexane acetic acid (compound of Formula (III)), may be more efficacious than the parent drug molecule (e.g., gabapentin or other GABA analog) in treating migraine, fibromyalgia, amyotrophic lateral sclerosis, irritable bowel syndrome, social phobia, Parkinson's disease, asthma, cough, or chronic obstructive pulmonary disease because the disclosed compounds require less time to reach a therapeutic concentration in the systemic circulation, i.e., the compounds disclosed herein have a shorter Tₘₐₓ than their parent drug counterparts when taken orally. It is believed that the compounds disclosed herein, for example, the gabapentin prodrug 1-{[(α-isobutanoyloxyethoxy)carbonyl]aminomethyl}-1-cyclohexane acetic acid (compound of Formula (III)), are absorbed from the gastrointestinal lumen into the blood by a different mechanism than that by which gabapentin and other known GABA analogs are absorbed. For example, gabapentin is believed to be actively transported across the gut wall by a carrier transporter localized in the human small intestine. The gabapentin transporter is easily saturated which means that the amount of gabapentin absorbed into the blood may not be proportional to the amount of gabapentin that is administered orally, because once the transporter is saturated, further absorption of gabapentin does not occur to any significant degree. In comparison to gabapentin, the compounds disclosed herein, for example, the gabapentin prodrug 1-{[(α-isobutanoyloxyethoxy)carbonyl]aminomethyl}-1-cyclohexane acetic acid (compound of Formula (III)), are believed to be absorbed across the gut wall along a greater portion of the gastrointestinal tract, including the colon.

Because the compounds disclosed herein can be effectively formulated in sustained release formulations, which provide for sustained release of a GABA analog prodrug into the gastrointestinal tract, for example, within the colon, over a period of hours, the compounds, such as the gabapentin prodrug 1-{[(α-isobutanoyloxyethoxy)carbonyl]aminomethyl}-1-cyclohexane acetic acid (compound of Formula (III)), may be more efficacious than their respective parent drugs (e.g., gabapentin or other GABA analog) in treating migraine, fibromyalgia, amyotrophic lateral sclerosis, irritable bowel syndrome, social phobia, Parkinson's disease, asthma, cough, or chronic obstructive pulmonary disease. The ability of the compounds provided by the present disclosure to be used in sustained release oral dosage forms may reduce the dosing frequency necessary for maintenance of a therapeutically effective drug concentration in the systemic circulation.

Dosage forms comprising a GABA analog prodrug provided by the present disclosure may be administered or applied singly or in combination with each other or with other agents. The dosage forms may also deliver a prodrug of a GABA analog to a patient in combination with another pharmaceutically active agent including another prodrug of a GABA analog and/or another active agent known or believed to be capable of treating migraine, fibromyalgia, amyotrophic lateral sclerosis, irritable bowel syndrome, social phobia, Parkinson's disease, asthma, cough, or chronic obstructive pulmonary disease.

In certain embodiments, GABA analog prodrugs may be suitable for oral administration. In certain embodiments, the promoiety or promoieties are cleaved after absorption of the GABA analog prodrug by the gastrointestinal tract (e.g., in intestinal tissue, blood, liver or other suitable tissue of the patient) following oral administration of the GABA analog prodrug. The promoiety or promoieties may render the prodrug a substrate for one or more transporters expressed in the large intestine (i.e., colon), and/or, for GABA analogs that are poorly absorbed across the gastrointestinal mucosa (e.g., gabapentin and pregabalin), and/or may facilitate the ability of the prodrug to be passively absorbed across the gastrointestinal mucosa.

Migraine is a neurological disorder that is characterized by recurrent attacks of headache, with pain most often occurring on one side of the head, accompanied by various combinations of symptoms such as nausea, vomiting, and sensitivity to light, sound, and odors. Migraine is estimated to affect 28 million Americans older than 12 years of age (˜10% of the U.S. population), with a 17.6% preponderance in females, a 5.7% in males. Migraine is familial and often hereditary, and is most common in women, particularly young adult women. Migraine is recognized as a chronic illness, not simply as a headache.

The exact mechanism of migraine initiation and progress is not known. Migraine can occur at any time of day or night, but occurs most frequently on arising in the morning. Migraine can be triggered by various factors, such as hormonal changes, stress, foods, lack of sleep, excessive sleep, or visual, auditory, olfactory, or somatosensory stimulation. In general, there are four phases to a migraine: the prodrome, auras, the attack phase, and postdrome. The prodrome phase is a group of vague symptoms that may precede a migraine attack by several hours, or even a few days before a migraine episode. Prodrome symptoms can include sensitivity to light and sound, changes in appetite, fatigue and yawning, malaise, mood changes, and food cravings. Auras are sensory disturbances that occur before the migraine attack in one in five patients. Positive auras include bright or shimmering light or shapes at the edge of the field of vision. Other positive aura experiences are zigzag lines or stars. Negative auras are dark holes, blind spots, or tunnel vision. Patients may have mixed positive and negative auras. Other neurologic symptoms that may occur at the same time as the aura include speech disturbances, tingling, numbness, or weakness in an arm or leg, perceptual disturbances such as space or size distortions, and confusion. A migraine attack usually lasts from 4 to 72 hours and typically produces throbbing pain on one side of the head, pain worsened by physical activity, nausea, visual symptoms, facial tingling or numbness, extreme sensitivity to light and noise, looking pale and feeling cold, and less commonly tearing and redness in one eye, swelling of the eyelid, and nasal congestion. During the attack the pain may migrate from one part of the head to another, and may radiate down the neck into the shoulder. Scalp tenderness occurs in the majority of patients during or after an attack. After a migraine attack, there is usually a postdrome phase, in which patients may feel exhausted, irritable, and/or be unable to concentrate. Other types of migraine include menstrual migraines, ophthalmologic migraine, retinal migraine, basilar migraine, familial hemiplegic migraine, and status migrainosus.

It is theorized that persons prone to migraine have a reduced threshold for neuronal excitability, possibly due to reduced activity of the inhibitory neurotransmitter γ-aminobutyric acid (GABA). GABA normally inhibits the activity of the neurotransmitters serotonin (5-HT) and glutamate, both of which appear to be involved in migraine attacks. The excitatory neurotransmitter glutamate is implicated in an electrical phenomenon called cortical spreading depression, which can initiate a migraine attack, while serotonin is implicated in vascular changes that occur as the migraine progresses.

There are a number of drugs that are currently available for prophylactic treatment of migraine, including propanolol, amitriptyline, valproate, verapamil, phenelzine, and methysergide, as well as aspirin-like drugs, such as aspirin, naproxen, ibuprofen, mefenamic acid, flufenamic acid, and tolfenamic acid. Typically, these drugs must be taken daily, and some are associated with severe adverse effects or the high dosage amounts required for effectiveness make them undesirable. The estimated probability of success with any one of these prophylactic anti-migraine drugs is about 60% to 75% (see, e.g., Harrison's Principles of Internal Medicine, eds. Isselbacher et al., McGraw-Hill, Inc., New York, p. 69 (1994)).

Many anticonvulsant compounds, e.g., topiramate, valproic acid, gabapentin, levetiracetuamm, carbamazepine, isovaleramide, and α-methyl isovaleramide used in the treatment of seizure disorders are also effective prophylactic migraine treatments. While the mechanism by which these anticonvulsant compounds alleviate migraine is not known, it is believed that the anticonvulsant compounds stabilize episodic phenomena from various biochemical or physical origins and generally result in a decrease in central nervous system (CNS) excitability. In particular, anti-epileptic drugs that increase brain levels of GABA, either by increasing GABA synthesis or reducing its breakdown, appear to be effective in preventing migraine in certain individuals. A number of studies have shown that gabapentin is useful for preventing migraine (see, e.g., Mathew et al., Headache 2001, 41, 119-128; Mathew, Cephalalgia 1996, 16, 367; Magnus-Miller et al., American Pain Society Program, 17th Annual Meeting, Abstract No. 645, San Diego, Calif., Nov. 5-8, 1998; and Wessely et al., Cephalalgia 1987, 7(Suppl 6), 477-478).

GABA analog prodrugs provided by the present disclosure or pharmaceutical composition thereof may be administered to a patient after initiation of the migraine. For example, a patient may be in the headache phase of the migraine or the postdrome phase before the prodrug or composition is administered. Alternatively, GABA analog prodrugs provided by the present disclosure or pharmaceutical composition thereof may be administered to the patient before the migraine starts, such as once the patient senses that a migraine is developing or when the early symptoms of the migraine have begun. GABA analog prodrugs provided by the present disclosure may also be administered to a patient on an ongoing or chronic basis to treat recurrent or frequent occurrences of migraine episodes. This is known as prophylactic treatment.

Migraine may be diagnosed by determining whether some of a person's recurrent headaches meet migraine criteria as disclosed in, for example, The International Classification of Headache Disorders, 2nd edition, Headache Classification Committee of the International Headache Society, Cephalalgia 2004, 24 (suppl 1).

Fibromyalgia is a condition characterized by aching and pain in muscles, tendons and joints all over the body, but especially along the spine. The body also is tender to touch in specific areas referred to as tender or trigger points. Other symptoms of fibromyalgia include sleep disturbance, depression, daytime tiredness, headaches, alternating diarrhea and constipation, numbness and tingling in the hands and feet, feelings of weakness, memory difficulties, and dizziness. Although the etiology of fibromyalgia is not known, stress, disordered sleep patterns, abnormal production of pain-related chemicals in the nervous system, and/or low levels of growth hormone are believed to contribute to the onset of fibromyalgia.

Fibromyalgia usually occurs in people between 20 and 60 years of age and is estimated to affect 3.4% of women and 0.5% of men. The incidence of juvenile primary fibromyalgia in school age girls is estimated to be about 1.2%.

Current treatment of fibromyalgia is based on symptoms, with the goal of alleviating pain, restoring sleep, and improving general quality of life. Several nonpharmacologic treatments include exercise, education, and behavioral and physical therapy. Pharmacologic treatments include tricyclic compounds, serotonin reuptake inhibitors, analgesics, muscle relaxants, and ACE inhibitors. Studies suggest that fibromyalgia antiepileptic drugs such as gabapentin and pregabalin are effective in treating fibromyalgia (see e.g., Nampiaparampil and Schmerling, Am J Manage Care 2004, 10, 794-800; Crofford, Curr Rheumatol Rep, 2004, 6, 274-80; Zareba, Drugs Today, 2005, 41(8), 509-516; and Dooley et al., U.S. Application Publication No. 2004/0180959). Pregabalin is an α₂-δ ligand that is shown to have analgesic, anxiolytic-like, and anticonvulsant activity in animal models (Crofford et al., Arthritis & Rheumatism, 2005, 52(4), 1264-1273). Doses of 300 or 450 mg/day of pregabalin have been shown to improve the quality of life by decreasing pain and fatigue (Crofford et al., Arthritis & Rheumatism, 2005, 52(4), 1264-1273).

GABA analog prodrugs provided by the present disclosure may be administered to a patient to treat fibromyalgia and diseases, disorders, and conditions associated with fibromyalgia. Treatment of fibromyalgia includes decreasing pain and fatigue, and increasing the patient's quality of life.

Amyotrophic lateral sclerosis (ALS), also known as Lou Gehrig's disease, is a rapidly progressive, usually fatal neurological disease that attacks the nerve cells responsible for controlling voluntary muscles. The disease belongs to a group of disorders known as motor neuron disease, which is characterized by the gradual degeneration and death of motor neurons. In ALS, both the upper motor neurons and the lower motor neurons degenerate or die, ceasing to send messages to muscles. Unable to function, the muscles gradually weaken, atrophy, and twitch. Eventually, the ability of the brain to start and control voluntary movement is lost. When muscles in the diaphragm and chest wall fail, patients lose the ability to breathe without ventilary support. Most people with ALS die from respiratory failure, usually within 3 to 5 years from the onset of symptoms.

About 20,000 persons in the United States have ALS, and an estimated 5,000 people are diagnosed with the disease each year. The etiology of ALS is not known.

Riluzole, the first drug approved by the FDA for treating ALS, is believed to reduce damage to motor neurons by decreasing the release of glutamate (see e.g., Hurko and Walsh, J Neurol Sci 2000, 180(1-2), 21-21; and Gordon, Curr Neurol Neurosci Rep 2005, 5(1), 48-54). Studies suggest that gabapentin is also effective in preventing neuronal cell death and therefore may be useful in the treatment of ALS (see, e.g., Taylor, Rev Neurol, 1997, 153(Suppl 1), S39-45; and Cory, Ann Pharmacother 1995, 29(11), 1160-61) although other studies have shown negative results (see e.g., Miller et al., Neurology, 2001, 56, 843-848).

GABA analog prodrugs provided by the present disclosure may be administered to a patient to treat ALS and diseases, disorders, and conditions associated with ALS.

Irritable bowel syndrome (IBS) is a disorder of bowel function (see e.g., Mertz, N Engl J Med 2003, 349, 2136-46). Patients suffering from IBS have changes in bowel habits such as constipation or diarrhea, and abdominal pain along with other gastrointestinal symptoms such as heartburn, premature satiety, nausea, abdominal fullness bloating, dyspepsia, and/or urgency. Non-gastrointestinal symptoms include fatigue, muscle aches and pain, fibromyalgia, headaches, back pain, sleep disturbances, sexual dysfunction, and urinary symptoms including urinary urgency, urinary hesitation or a feeling of spasm in the bladder. IBS is estimated to affect between 15% and 20% of the United States population. The symptoms of IBS are believed to be produced by abnormal function of the nerves and muscles in the bowel to cause the bowel to become irritated or overly sensitive to stimuli. Other factors postulated to contribute to IBS include infection, immune modulation, and inflammation, as well as genetic factors (see e.g., Gilkin, Clin Ther 2005, 27(11), 1696-709; Quigley, World J Gastroenterol 2006, 12(1), 1-5; Mayer and Collins, Gastroenterology 2002, 122(7), 2032-48; and Mayer et al., Dig Dis 2001, 19(3), 212-218).

Based on positive results from clinical studies (see e.g., Lee et al., Aliment Pharmacol Ther 2005, 22(10), 981-988), the use of gabapentin and other GABA analogs for treating IBS has been proposed (see e.g., Bryans et al., WO 00/50027, Bueno et al., U.S. Pat. No. 6,127,418; Gaeta et al., U.S. Application Publication No. 2003/0119756; and Gaeta and Cintron, U.S. Application Publication No. 2003/0119756). Doses of gabapentin up to 600 mg/day have been shown to be effective in treating IBS (Lee et al., Aliment Pharmacol Ther 2005, 22, 291-988).

GABA analog prodrugs provided by the present disclosure may be administered to a patient to treat IBS and diseases, disorders, and conditions associated with IBS.

Social phobia, also termed social anxiety disorder, is a common psychiatric illness that imposes persistent functional impairment and disability on persons having the disorder. Social phobia is characterized by extreme anxiety in social and performance situations that include fear of humiliation, embarrassment, or scrutiny by other people (American Psychiatric Association, Diagnostic and Statistical Manual of Mental Disorders, DSM-IV-TR, American Psychiatry Association, Washington D.C., 2000). In global social phobia, all social situations are poorly tolerated, while specific social phobia includes performance anxiety or well-delineated phobia. Because of the persistent fear, social interactions or performance situations are either avoided or endured with intense discomfort, significantly interfering with normal routine or life functioning.

Social phobia is estimated to affect 13% to 16% of the population with a mean age of onset during the critical period between 11 and 15 years of age (see e.g., Connor et al., Epilepsia, 1999, 40 (Suppl. 6), S60-S65; Li et al., J Psychiatry Neurosci 2001, 26(3), 190-202. Social phobia tends to be chronic, affecting all areas of a person's life, and is associated with significantly increased co-morbidity. For example, persons having social phobia are more likely to also experience simple phobia, agoraphobia alcohol abuse, major depression, generalized anxiety, and panic disorder, and to attempt suicide. If left untreated, social phobia will become associated with extensive morbidity and disability, leading to lifelong impairment in social development and occupational functioning.

Deregulation of neurotransmitter function in the brain, including the noradrenaline, serotonin, γ-aminobutyric acid, and dopamine neurotransmitter systems, is thought to play a role in social phobia (see e.g., Millan, Progress in Neurobiology, 2003, 70, 83-244; and Li et al., J Psychiatry Neurosci 2001, 26(3), 190-202). Clinical studies have shown gabapentin to be effective in treating social phobia as measured by several social phobia scales (Pande et al., J Clin Psychiatry 1994, 54 Suppl), 31-35; Pande et al., J Clin Psychopharrnacol 1999, 19, 341-348; and Pande et al., J Clin Psychopharmacol 2000, 20(5), 544-546). Other studies have shown the therapeutic efficacy of pregabalin for treating social phobia (see e.g., Pande et al., J Clin Psychopharmacol 2004, 24(2), 141-149; Selak, Curr Opin Investig Drugs 2001, 2(6), 828-834; Lauria-Horner and Pohl, Expert Opin Investig Drugs 2003, 12(4), 663-672; Kasper et al., Eur Neuropsychopharmacol 2002, 12 (Suppl), S341-S342; Rickels et al., 2002, Int J Neuropsychopharmacol 2002, 5, 14-15; and Smith et al., Eur Neuropsychopharmacol 2002, 12, S350). It is hypothesized that gabapentin and pregabalin mediate their anxiolyitic actions by interacting with α₂/δ₁ and α₂/δ₂ subunits of voltage-dependent calcium channels, potentially by suppressing noradrenaline release via a reduction of glutamate release (see Millan, Progres in Neurobiology, 2003, 70, pp. 107-108 and 165, and references therein).

GABA analog prodrugs provided by the present disclosure may be administered to a patient to treat social phobia and diseases, disorders, and conditions associated with social phobia. Doses of gabapentin from 600-3600 mg per day have been shown effective in alleviating the symptoms of social phobia (Pande et al., J Clin Psychiatry 1994, 54 (Suppl), S31-S35; Pande et al., J Clin Psychopharmacol 1999, 19, 341-348; and Pande et al., J Clin Psychopharmacol 2000, 20(5), 544-546).

Social phobia may be diagnosed using, for example, any of the following tests: the Liebowitz Social Anxiety Scale (LSAS), the CGI-Severity of Illness scale, the Hamilton Rating Scale for Anxiety (HAM-A), the Hamilton Rating Scale for Depression (HAM-D), the axis V Social and Occupational Functioning Assessment Scale of DSM-IV, the axis II (ICD10) World Health Organization Disability Assessment, Schedule 2 (DAS-2), the Sheehan Disability Scales, the Schneier Disability Profile, or the World Health Organization Quality of Life-100 (WHOQOL-100).

Parkinson's disease (PD) is a progressive neurodegenerative disorder that affects about 1% of the population over 55 years of age. The pathological manifestation of PD is the loss of dopaminergic neurons in the Substantia Nigra pars compacta and the presence of intracytoplasmic inclusions, called Lewy bodies, formed mainly by α-synuclein and ubiquitin. The main symptoms of PD are tremor, bradykinesia, hypokinesia, and balance and coordination disturbances. Dopamine replacement therapy can alleviate the symptoms of PD, however as the disease progresses, drug-related side effects emerge as well as disabling symptoms that are not responsive to the treatment. Although the cause of PD is unknown, dopaminergic cell loss has been associated with several mechanisms of cell damage including excitotoxicity, disturbed calcium homeostasis, inflammation, apoptosis, distress energy metabolism, and protein aggregation. Because patients with PD have a normal lifespan, they must endure crippling symptoms for many years, severely impacting their quality of life. Therefore, a neuroprotective therapy that can stop or reduce the continual loss of dopaminergic neurons is needed.

Clinical studies suggest that gabapentin is useful in improving rigidity, bradykinesia, and tremor associated with PD (see e.g., Olson et al., Am J. Med 1997, 102(1), 60-6; Faulkner et al., Ann Pharmacother 2003, 37(2), 282-286; Marjama-Lyons and Koller, Drugs Aging 2000, 16(4), 273-278; and Van Blercom et al., Clin Neuropharmacol 2004, 27(3), 124-128).

GABA analog prodrugs provided by the present disclosure may be administered to a patient to treat Parkinson's disease and diseases, disorders, and conditions associated with Parkinson's disease. Doses of gabapentin up to 1800 mg/day have been shown to be effective in treating tremors associated with PD (Faulkner et al., Ann Pharmacother 2003, 37(2), 282-286).

Cough reflex, elicited by activation of cough receptors located in the respiratory tract, clears inhaled irritants and foreign substances from the respiratory tract and in conjunction with the mucociliary system can expel excessive airway secretion produced under abnormal conditions from the respiratory tract. Cough can be caused by mild acuate upper respiratory tract infections, allergies, asthma, chronic obstructive pulmonary disease, lung cancer, gastroesophageal reflux disease, post-nasal drip, and heart or ear disorders. However, chronic non-productive cough having no identifiable cause accounts for a significant percent of patients presenting with cough. Chronic cough is associated with exacerbation of asthmatic symptoms, rib fractures, breathlessness, ruptured abdominal muscles, pneumothorax, syncope, second and third degree heart block, and loss of consciousness. Persistent and uncontrollable cough can lead to morbidity and severely impairs the quality of life of these patients.

Gabapentin at doses of 100 mg/day to 900 mg/day has been shown to be effective in treating chronic cough or throat-clearing as a manifestation of sensory neuropathy involving the superior or recurrent laryngeal nerve (Lee and Woo, Ann Oto Rhinol Laryngol 2005, 114(4), 253-7), and in a one-year study in which 200 mg/day to 16000 mg/day was administered to patients with idiopathic chronic cough (Mintz and Lee, Am J Med 2006, 119, e13-e15). It has been proposed that sensory receptors and pathways regulating cough are abnormally regulated in chronic cough (Mazzone, Cough 2005, 1(2); and Bastian et al., Otol—Head Neck Surgery 2006, 135, 17-21). It is also suggested that GABA analogs may be used to treat pulmonary diseases such as cough and chronic obstructive pulmonary disease as substance P modulating agents (Magistro, International Publication No. WO 00/67742; and). Thus, GABA analog prodrugs provided by the present disclosure may be administered to a patient to treat cough and diseases, disorders, and conditions associated with cough, and may be particularly useful in the treatment of cough in which there is an allodynia-like pathology in the laryngeal and bronchial nerves.

Asthma is reversible airway obstruction in which the airway occasionally constricts becomes inflamed, and is lined with an excessive amounts of mucus. Symptoms of asthma include dyspnea, wheezing, chest tightness, and cough. Asthma episodes may be induced by airborne allergens, food allergies, medications, inhaled irritants, physical exercise, respiratory infection, psychological stress, hormonal changes, cold weather, or other factors. One of the characteristic features of asthma is the propensity of the airways to respond to stimuli that are otherwise innocuous to healthy subjects, and the similarities in the mechanisms contributing to hyperalgesia and allodynia in clinical pain syndromes and bronchial hyperresponsiveness in asthma has been recognized (Spina, Pulm Pharmacol Ther 2003, 16(1), 31-44). GABA analogs such as gabapentin and pregabalin are known to be effective in treating neuropathic pain and therefore can be expected to be effective in treating asthma associated with hyperalgesia and allodynia. Therefore, GABA analog prodrugs provided by the present disclosure may be administered to a patient to treat asthma and diseases, disorders, and conditions associated with asthma.

Chronic obstructive pulmonary disease (COPD), also known as chronic obstructive airway disease, is a group of diseases characterized by the pathological limitation of airflow in the airway that is not fully reversible, and includes conditions such as chronic bronchitis, emphysema, as well as other lung disorders such as asbestosis, pneumoconiosis, and pulmonary neoplasms (see, e.g., Barnes, Pharmacological Reviews, 2004, 56(4), 515-548). The airflow limitation is usually progressive and associated with an abnormal inflammatory response of the lungs to noxious particles and gases. COPD is characterized by a shortness of breath the last for months or years, possibly accompanied by wheezing, and a persistent cough with sputum production. COPD is most often caused by tobacco smoking, although it can also be caused by other airborne irritants such as coal dust, asbestos, urban pollution, or solvents. COPD encompasses chronic obstructive bronchiolitis with fibrosis and obstruction of small airways, and emphysema with enlargement of airspaces and destruction of lung parenchyma, loss of lung elasticity, and closure of small airways.

It has been proposed that GABA analogs such as gabapentin and pregabalin can be useful in treating chronic obstructive pulmonary disease (Bertrand, U.S. Application Publication No. 2004/0143014; and Magistro, WO 00/67742. Therefore, GABA analog prodrugs provided by the present disclosure may be administered to a patient to treat chronic obstructive pulmonary disease and diseases, disorders, and conditions associated with chronic obstructive pulmonary disease. Other pulmonary diseases, disorders, or conditions for which GABA analog prodrugs provided by the present disclosure can be useful in treating include chronic or acute chronic or acute bronchoconstriction, large airway obstruction, chronic bronchitis, small airway obstruction, and emphysema; pneuomconiosis such as aluminosis, anthracosis, asbestosis, chalicosis, ptilosis, siderosis, silicosis, byssinosis, and talc pneumoconiosis; bronchitis such as acute bronchitis, acute laryngotracheal bronchitis, arachidic bronchitis, catarrhal bronchitis, cropus bronchitis, dry bronchitis, infectious asthmatic bronchitis, productive bronchitis, staphylococcus or streptococcal bronchitis, and vesicular bronchitis; and bronchiectasis such as cylindric bronchiectasis, sacculated bronchiectasis, fusiform bronchiectasis, capillary bronchiectasis, cystic bronchiectasis, dry bronchiectasis, and follicular bronchiectasis

### Pharmaceutical Compositions

Pharmaceutical compositions provided by the present disclosure comprise at least one compound of Formula (I), Formula (II), Formula (III), and/or Formula (IV) and at least one pharmaceutically acceptable vehicle. A pharmaceutical composition may comprise a therapeutically effective amount of compound of Formula (I), Formula (II), Formula (III), and/or Formula (IV) and at least one pharmaceutically acceptable vehicle. In certain embodiments, a pharmaceutical composition may comprise more than one compound of Formula (I), Formula (II), Formula (III), and/or Formula (IV). Pharmaceutically acceptable vehicles include diluents, adjuvants, excipients, and carriers.

Pharmaceutical compositions may be produced using procedures and methods known in the art. For example, pharmaceutical compositions may be manufactured by means of conventional mixing, dissolving, granulating, dragee-making, levigating, emulsifying, encapsulating, entrapping, or lyophilizing processes. Pharmaceutical compositions may be formulated using one or more physiologically acceptable carriers, diluents, excipients, or auxiliaries, which facilitate processing of compounds disclosed herein into preparations, which can be used pharmaceutically. Proper formulation can depend, in part, on the route of administration.

Pharmaceutical compositions provided by the present disclosure may provide therapeutic or prophylactic levels of a GABA analog upon administration to a patient. The promoiety or promoieties of a GABA analog prodrug may be cleaved in vivo either chemically and/or enzymatically to release the corresponding GABA analog. One or more enzymes present in the intestinal lumen, intestinal tissue, blood, liver, brain, or any other suitable tissue of a mammal may enzymatically cleave the promoiety or promoieties of the administered prodrugs. For example, the promoiety or promoieties may be cleaved after absorption from the gastrointestinal tract (e.g., in intestinal tissue, blood, liver, or other suitable tissue of a mammal). In certain embodiments, a GABA analog remains conjugated to the promoiety or promoieties during transit across the intestinal mucosal barrier to provide protection from presystemic metabolism. In certain embodiments, a GABA analog prodrug is essentially not metabolized to release the corresponding GABA analog within enterocytes, but is metabolized to the parent drug within the systemic circulation. Cleavage of the promoiety or promoieties of a GABA analog prodrug after absorption by the gastrointestinal tract may allow the prodrug to be absorbed into the systemic circulation either by active transport, passive diffusion, or by a combination of both active and passive processes.

GABA analog prodrugs may remain intact until after passage of the prodrug through a biological barrier, such as the blood-brain-barrier. In certain embodiments, prodrugs provided by the present disclosure may be partially cleaved, e.g., one or more, but not all, of the promoieties can be cleaved before passage through a biological barrier or prior to being taken up by a cell, tissue, or organ.

GABA analog prodrugs may remain intact in the systemic circulation and be absorbed by cells of an organ, either passively or by active transport mechanisms. In certain embodiments, a GABA analog prodrug will be lipophilic and can passively translocate through cellular membranes. Following cellular uptake, the GABA analog prodrug may be cleaved chemically and/or enzymatically to release the corresponding GABA analog into the cellular cytoplasm, resulting in an increase in the intracellular concentration of the GABA analog.

In certain embodiments, a pharmaceutical composition may comprise at least one compound of Formula (I), Formula (II), Formula (III), or Formula (IV) in an amount effective for the treatment of migraine, fibromyalgia, amyotrophic lateral sclerosis, irritable bowel syndrome, social phobia, Parkinson's disease, asthma, cough, and chronic obstructive pulmonary disease, in a patient.

In certain embodiments, a pharmaceutical composition may include an adjuvant that facilitates absorption of at least one compound of Formula (I); Formula (II), Formula (III), or Formula (IV) through the gastrointestinal epithelia. Such enhancers may, for example, open the tight-junctions in the gastrointestinal tract or modify the effect of cellular components, such as p-glycoprotein and the like. Suitable enhancers can include alkali metal salts of salicylic acid, such as sodium salicylate, caprylic or capric acid, such as sodium caprylate or sodium caprate, and the like. Enhancers can include, for example, bile salts, such as sodium deoxycholate. Various p-glycoprotein modulators are described in Fukazawa et al., U.S. Pat. No. 5,112,817 and Pfister et al., U.S. Pat. No. 5,643,909. Various absorption enhancing compounds and materials are described in Burnside et al., U.S. Pat. No. 5,824,638, and Meezam et al., U.S. Application Publication No. 2006/0046962. Other adjuvants that enhance permeability of cellular membranes include resorcinol, surfactants, polyethylene glycol, and bile acids.

In certain embodiments, a pharmaceutical composition can include an adjuvant that reduces enzymatic degradation of at least one compound of Formula (I), Formula (II), Formula (III), or Formula (IV). Microencapsulation using protenoid microspheres, liposomes, or polysaccharides can also be effective in reducing enzymatic degradation of administered compounds

A pharmaceutical composition may also include one or more pharmaceutically acceptable vehicles, including excipients, adjuvants, carriers, diluents, binders, lubricants, disintegrants, colorants, stabilizers, surfactants, fillers, buffers, thickeners, emulsifiers, wetting agents, and the like. Vehicles may be selected to alter the porosity and permeability of a pharmaceutical composition, alter hydration and disintegration properties, control hydration, enhance manufacturability, etc.

In certain embodiments, a pharmaceutical composition may be formulated for oral administration. Pharmaceutical compositions formulated for oral administration may provide for uptake of at least one compound of Formula (I), Formula (II), Formula (III), or Formula (IV) throughout the gastrointestinal tract, or in a particular region or regions of the gastrointestinal tract. In certain embodiments, a pharmaceutical composition may be formulated to enhance uptake of at least one compound of Formula (I), Formula (II), Formula (III), or Formula (IV) from the lower gastrointestinal tract, and in certain embodiments, from the large intestine, including the colon. Such compositions may be prepared in a manner known in the pharmaceutical art and may further comprise, in addition to at least one compound of Formula (I), Formula (II), Formula (III), and Formula (IV), one or more pharmaceutically acceptable vehicles, permeability enhancers, and/or a second therapeutic agent.

In certain embodiments, a pharmaceutical composition may further comprise substances to enhance, modulate and/or control release, bioavailability, therapeutic efficacy, therapeutic potency, stability, and the like. For example, to enhance therapeutic efficacy of at least one compound of Formula (I), Formula (II), Formula (III), or Formula (IV) may be co-administered with one or more active agents to increase the absorption or diffusion of at least one compound of Formula (I), Formula (II), Formula (III), or Formula (IV) from the gastrointestinal tract, or to inhibit degradation of the drug in the systemic circulation. In certain embodiments, at least one compound of Formula (I), Formula (II), Formula (III), or Formula (IV) may be co-administered with active agents having pharmacological effects that enhance the therapeutic efficacy of at least one compound of Formula (I), Formula (II), Formula (III), or Formula (IV).

Pharmaceutical compositions may take the form of solutions, suspensions, emulsions, tablets, pills, pellets, capsules, capsules containing liquids, powders, sustained-release formulations, suppositories, emulsions, aerosols, sprays, mists, suspensions, or any other form suitable for use.

Pharmaceutical compositions comprising at least one compound of Formula (I), Formula (II), Formula (III), or Formula (IV) may be formulated for oral administration. Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame and/or saccharin, flavoring agents such as peppermint, oil of wintergreen, cherry, or other suitable flavorings, coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions may include standard vehicles such as mannitol, lactose, starch, magnesium stearate, sodium saccharine, cellulose, magnesium carbonate, etc. Such vehicles may be of pharmaceutical grade.

For oral liquid preparations such as, for example, suspensions, elixirs, and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines, and the like may be added.

When the compound of Formula (I), Formula (II), Formula (III), or Formula (IV) is acidic, it may be included in any of the formulations provided by the present disclosure as the free acid, a pharmaceutically acceptable salt, a solvate, or a hydrate. Pharmaceutically acceptable salts substantially retain the activity of the free acid, may be prepared by reaction with bases, and tend to be more soluble in aqueous and other protic solvents than the corresponding free acid form. In some embodiments, sodium salts of at least one compound of Formula (I), Formula (II), Formula (III), or Formula (IV) can be used in a formulation.

Pharmaceutical compositions provided by the present disclosure may be formulated for parenteral administration including administration by injection, for example, into a vein (intravenously), an artery (intraarterially), a muscle (intramuscularly), under the skin (subcutaneously or in a depot formulation), to the pericardium, to the coronary arteries, or used as a solution for delivery to a tissue or organ, for example, use in a cardiopulmonary bypass machine or to bathe transplant tissues or organs. Injectable compositions may be pharmaceutical compositions for any route of injectable administration, including, but not limited to, intravenous, intrarterial, intracoronary, pericardial, perivascular, intramuscular, subcutaneous, intradermal, intraperitoneal, and intraarticular. In certain embodiments, an injectable pharmaceutical composition may be a pharmaceutically appropriate composition for administration directly into the heart, pericardium, or coronary arteries.

Pharmaceutical compositions provided by the present disclosure suitable for parenteral administration may comprise one or more compounds of Formula (I), Formula (II), Formula (III), or Formula (IV) in combination with one or more pharmaceutically acceptable sterile isotonic aqueous, water-miscible, or non-aqueous vehicles. Pharmaceutical compositions for parenteral use may include substances that increase and maintain drug solubility such as complexing agents and surface acting agents, compounds that make the solution isotonic or near physiological pH such as sodium chloride, dextrose, and glycerin, substances that enhance the chemical stability of a solution such as antioxidants, inert gases, chelating agents, and buffers, substances that enhance the chemical and physical stability, substances that minimize self aggregation or interfacial induced aggregation, substances that minimize protein interaction with interfaces, preservatives including antimicrobial agents, suspending agents, emulsifying agents, and combinations of any of the foregoing. Pharmaceutical compositions for parenteral administration can be formulated as solutions, suspensions, emulsions, liposomes, microspheres, nanosystems, and powder to be reconstituted as solutions.

For prolonged delivery, a pharmaceutical composition may be provided as a depot preparation, for administration by implantation, e.g., subcutaneous, intradermal, or intramuscular injection. Thus, in certain embodiments, a pharmaceutical composition may be formulated with suitable polymeric or hydrophobic materials, e.g., as an emulsion in a pharmaceutically acceptable oil, ion exchange resins, or as a sparingly soluble derivative, e.g., as a sparingly soluble salt form of a compound of Formula (I), Formula (II), Formula (III), or Formula (IV).

Pharmaceutical compositions provided by the present disclosure may be formulated so as to provide immediate, sustained, or delayed release of a compound of Formula (I), Formula (H), Formula (III), or Formula (IV) after administration to a patient by employing procedures known in the art (see, e.g., Allen et al., “Ansel's Pharmaceutical Dosage Forms and Drug Delivery Systems,” 8th edition, Lippincott, Williams & Wilkins, August 2004). In certain embodiments, a pharmaceutical composition comprising at least one compound of Formula (I), Formula (II), Formula (III), or Formula (IV) may be formulated for sustained release formulation.

Furthermore, pharmaceutical compositions provided by the present disclosure and as disclosed herein may be formulated so as to contain any combination of at least one compound of Formula (I), Formula (II), Formula (III), or Formula (IV).

### Dosage Forms

Pharmaceutical compositions provided by the present disclosure may be formulated in a unit dosage form. A unit dosage form refers to a physically discrete unit suitable as a unitary dose for patients undergoing treatment, with each unit containing a predetermined quantity of at least one compound of Formula (I), Formula (II), Formula (III), or Formula (IV) calculated to produce the intended therapeutic effect. A unit dosage form may be for a single daily dose, 1 to 2 times per day, or one of multiple daily doses, e.g., 2 to 4 times per day. When multiple daily doses are used, a unit dosage may be the same or different for each dose. One or more dosage forms may comprise a dose, which may be administered to a patient at a single point in time or during a time interval.

Pharmaceutical compositions provided by the present disclosure may be used in dosage forms that provide immediate release and/or controlled release of at least one compound of Formula (I), Formula (II), Formula (III), or Formula (IV). The appropriate type of dosage form can depend on the type or severity of migraine, fibromyalgia, amyotrophic lateral sclerosis, irritable bowel syndrome, social phobia, Parkinson's disease, asthma, cough, or chronic obstructive pulmonary disease being treated, and on the method of administration. In certain embodiments, dosage forms may be adapted to be administered to a patient no more than twice per day, and in certain embodiments, only once per day. Dosing may be provided alone or in combination with other drugs and may continue as long as required for effective treatment of the disease, disorder, or condition.

Pharmaceutical compositions comprising at least one compound of Formula (I), Formula (II), Formula (III), or Formula (IV) may be formulated for immediate release for parenteral administration, oral administration, or by any other appropriate route of administration.

Controlled drug delivery systems may be designed to deliver a drug in such a way that the drug level is maintained within a therapeutically effective blood concentration range and effective and safe blood levels are maintained for a period as long as the system continues to deliver the drug at a particular rate. Controlled drug delivery may produce substantially constant blood levels of a drug as compared to fluctuations observed with immediate release dosage forms administered by the same route of administration. For some drugs, maintaining a constant blood and tissue concentration throughout the course of therapy is the most desirable mode of treatment. Immediate release of these drugs may cause blood levels to peak above the level required to elicit the desired response, which wastes the drug and may cause or exacerbate toxic side effects. Controlled drug delivery may result in optimum therapy, and not only may reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

In certain embodiments, oral dosage forms provided by the present disclosure may be controlled release dosage forms. Controlled delivery technologies can improve the absorption of a drug in a particular region or regions of the gastrointestinal tract. The appropriate oral dosage form for a particular pharmaceutical composition provided by the present disclosure can depend, at least in part, on the gastrointestinal absorption properties of the compound of Formula (I), Formula (II), Formula (III), or Formula (IV), the stability of the compound of Formula (I), Formula (II), Formula (III), or Formula (IV) in the gastrointestinal tract, the pharmacokinetics of the compound of Formula (I), Formula (II), Formula (III), or Formula (IV), and the intended therapeutic profile of the corresponding GABA analog. An appropriate controlled release oral dosage form may be selected for a particular compound of Formula (I), Formula (II), Formula (III), or Formula (IV). For example, gastric retention oral dosage forms may be appropriate for compounds absorbed primarily from the upper gastrointestinal tract, and sustained release oral dosage forms may be appropriate for compounds absorbed primarily from the lower gastrointestinal tract.

Pharmaceutical compositions provided by the present disclosure may be practiced with a number of different dosage forms, which can be adapted to provide sustained release of at least one compound of Formula (I), Formula (II),

Formula (III), or Formula (IV) upon oral administration. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

In diffusion-controlled systems, a water-insoluble polymer controls the flow of fluid and the subsequent egress of dissolved drug from the dosage form. Both diffusional and dissolution processes are involved in release of drug from the dosage form. In reservoir devices, a core comprising a drug is coated with the polymer, and in matrix systems, the drug is dispersed throughout the matrix. Cellulose polymers such as ethylcellulose or cellulose acetate can be used in reservoir devices. Examples of materials useful in matrix systems include methacrylates, acrylates, polyethylene, acrylic acid copolymers, polyvinylchloride, high molecular weight polyvinylalcohols, cellulose derivates, and fatty compounds such as fatty acids, glycerides, and carnauba wax.

In dissolution-controlled systems, the rate of dissolution of the drug is controlled by slowly soluble polymers or by microencapsulation. Once the coating is dissolved, the drug becomes available for dissolution. By varying the thickness and/or the composition of the coating or coatings, the rate of drug release can be. controlled. In some dissolution-controlled systems, a fraction of the total dose can comprise an immediate-release component. Dissolution-controlled systems include encapsulated/reservoir dissolution systems and matrix dissolution systems. Encapsulated dissolution systems can be prepared by coating particles or granules of drug with slowly soluble polymers of different thickness or by microencapsulation. Examples of coating materials useful in dissolution-controlled systems include gelatin, carnauba wax, shellac, cellulose acetate phthalate, and cellulose acetate butyrate. Matrix dissolution devices can be prepared, for example, by compressing a drug with a slowly soluble polymer carrier into a tablet form.

The rate of release of drug from osmotic pump systems is determined by the inflow of fluid across a semipermeable membrane into a reservoir, which contains an osmotic agent. The drug is either mixed with the agent or is located in a reservoir. The dosage form contains one or more small orifices from which dissolved drug is pumped at a rate determined by the rate of entrance of water due to osmotic pressure. As osmotic pressure within the dosage form increases, the drug is released through the orifice(s). The rate of release is constant and can be controlled within tight limits yielding relatively constant plasma and/or blood concentrations of the drug. Osmotic pump systems can provide a constant release of drug independent of the environment of the gastrointestinal tract. The rate of drug release can be modified by altering the osmotic agent and/or the size of the one or more orifices.

The release of drug from erosion-controlled systems is determined by the erosion rate of a carrier matrix. Drug is dispersed throughout the polymer and the rate of drug release depends on the erosion rate of the polymer. The drug-containing polymer may degrade from the bulk and/or from the surface of the dosage form.

Sustained release oral dosage forms may be in any appropriate form for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, and the like.

In certain embodiments, sustained release oral dosage forms may comprise a therapeutically effective amount of at least one compound of Formula (I), Formula (II), Formula (III), or Formula (IV) and a pharmaceutically acceptable vehicle. In certain embodiments, sustained release oral dosage forms may comprise less than a therapeutically effective amount of at least one compound of Formula (I), Formula (II), Formula (III), or Formula (IV) a pharmaceutically effective vehicle. Multiple sustained release oral dosage forms, each dosage form comprising less than a therapeutically effective amount of at least one compound of Formula (I), Formula (II), Formula (III), or Formula (IV), may be administered at a single time or over a period of time to provide a therapeutically effective dose or regimen for treating migraine, fibromyalgia, amyotrophic lateral sclerosis, irritable bowel syndrome, social phobia, Parkinson's disease, asthma, cough, or chronic obstructive pulmonary disease.

Sustained release oral dosage forms provided by the present disclosure may release at least one compound of Formula (I), Formula (II), Formula (III), or Formula (IV) from the dosage form to facilitate the ability of the compound of Formula (I), Formula (II) Formula (III), or Formula (IV) to be absorbed from an appropriate region of the gastrointestinal tract, for example, in the colon. In certain embodiments, sustained release oral dosage forms release at least one compound of Formula (I), Formula (II), Formula (III), or Formula (IV) from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms release at least one compound of Formula (I), Formula (II), Formula (III), or Formula (IV) from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 24 hours. In certain embodiments, sustained release oral dosage forms release at least one compound of Formula (I), Formula (II), Formula (III), or Formula (IV) from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 20 hours. In certain embodiments, sustained release oral dosage forms release at least one compound of Formula (I), Formula (II), Formula (III), or Formula (IV) from the dosage form in a delivery pattern of from about 0 wt % to about 20 wt % in about 0 to about 2 hours, about 20 wt % to about 50 wt % in about 0 to about 4 hours, about 55 wt % to about 85 wt % in about 0 to about 7 hours, and about 80 wt % to about 100 wt % in about 0 to about 8 hours.

Sustained release oral dosage forms comprising at least one compound of Formula (I), Formula (II), Formula (III), or Formula (IV) may provide a concentration of the corresponding GABA analog in the plasma, blood, or tissue of a patient over time, following oral administration to the patient. The concentration profile of a GABA analog may exhibit an AUC that is proportional to the dose of the corresponding compound of Formula (I), Formula (II), Formula (III), or Formula (IV).

Regardless of the specific form of controlled release oral dosage form used, a compound of Formula (I), Formula (II), Formula (III), or Formula (IV) may be released from an orally administered dosage form over a sufficient period of time to provide prolonged therapeutic concentrations of the compound of Formula (I), or Formula (II), Formula (III), or Formula (IV) in the plasma and/or blood of a patient that is effective for treating migraine, fibromyalgia, amyotrophic lateral sclerosis, irritable bowel syndrome, social phobia, Parkinson's disease, asthma, cough, or chronic obstructive pulmonary disease. Following oral administration, an oral dosage form comprising at least one compound of Formula (I), or Formula (II), Formula (III), and Formula (IV) can provide a therapeutically effective concentration of the corresponding GABA analog in the plasma and/or blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous time periods during which a therapeutically effective concentration of a GABA analog is maintained may be the same or different. The continuous period of time during which a therapeutically effective plasma concentration of a GABA analog is maintained may begin shortly after oral administration or after a time interval.

In certain embodiments, dosage forms can release from about 0% to about 30% of the prodrug in from about 0 hours to about 2 hours, from about 20% to about 50% of the prodrug in from about 2 hours to about 12 hours, from about 50% to about 85% of the prodrug in from 3 hours to about 20 hours and greater than about 75% of the prodrug in from about 5 hours to about 18 hours. In certain embodiments, sustained release oral dosage forms can provide a concentration profile of a GABA analog in the blood and/or plasma of a patient over time, which has an area under the curve (AUC) that is proportional to the dose of the GABA analog prodrug-administered, and a maximum concentration Cₘₐₓ. In certain embodiments, the Cₘₐₓ may be less than about 75%, and in certain embodiments, may be less than about 60%, of the Cₘₐₓ obtained from administering an equivalent dose of the compound from an immediate release oral dosage form and the AUC is substantially the same as the AUC obtained from administering an equivalent dose of the prodrug from an immediate release oral dosage form.

In certain embodiments, dosage forms may be administered twice per day, and in certain embodiments, once per day, to provide a therapeutically effective concentration of a GABA analog, e.g., gabapentin or pregabalin, in the systemic circulation of a patient.

Examples of sustained release oral dosage forms of GABA analogs are disclosed in Cundy et al., U.S. Pat. No. 6,833,140, U.S. Application Publication Nos. 2004/0198820 and 2006/0141034, each of which is incorporated by reference herein in its entirety.

### Methods of Administration and Doses

Methods for the treatment a disease chosen from migraine, fibromyalgia, amyotrophic lateral sclerosis, irritable bowel syndrome, social phobia, Parkinson's disease, asthma, cough, or chronic obstructive pulmonary disease comprise administering at least one GABA analog prodrug of Formula (I), Formula (II), Formula (III), or Formula (IV), or a pharmaceutical composition of any of the foregoing, to a patient in need of such treatment.

A compound of Formula (I), Formula (II), Formula (III), or Formula (IV), a pharmaceutically acceptable salt of any of the foregoing, a pharmaceutically acceptable solvate of any of the foregoing, or a pharmaceutical composition thereof may be administered by any appropriate route. Examples of suitable routes of administration include, but are not limited to, intradermal, intramuscular, intraperitoneal, intravenous, subcutaneous, intranasal, epidural, oral, sublingual, intracerebral, intravaginal, transdermal, rectal, inhalation, or topical. Administration may be systemic or local. Administration may be bolus injection, continuous infusion, or by absorption through epithelial or mucocutaneous linings, e.g., oral mucosa, rectal, and intestinal mucosa, etc. Administration may be systemic or local. In certain embodiments, at least one GABA analog prodrug of Formula (I), Formula (II), Formula (III), or Formula (IV), or pharmaceutical composition of any of the foregoing may be administered orally.

In certain embodiments, the at least one GABA analog prodrug of Formula (I), Formula (II), Formula (III), or Formula (IV), or pharmaceutical compositions of any of the foregoing can be delivered to a patient via sustained release dosage forms, for example, via oral sustained release dosage forms. When used to treat migraine, fibromyalgia, amyotrophic lateral sclerosis, irritable bowel syndrome, social phobia, Parkinson's disease, asthma, cough, or chronic obstructive pulmonary disease a therapeutically effective amount of one or more GABA analog prodrugs of Formula (I), Formula (II), Formula (III), or Formula (IV) can be administered or applied singly or in combination with other agents. A therapeutically effective amount of one or more GABA analog prodrugs of Formula (I), Formula (II), Formula (III), or Formula (IV) can also deliver a GABA analog prodrug provided by the present disclosure in combination with another pharmaceutically active agent, including another compound provided by the present disclosure. For example, in the treatment of a patient suffering from migraine, fibromyalgia, amyotrophic lateral sclerosis, irritable bowel syndrome, social phobia, Parkinson's disease, asthma, cough, or chronic obstructive pulmonary disease, a dosage form comprising a GABA analog prodrug of Formula (I), Formula (II), Formula (III), and/or Formula (IV) can be administered in conjunction with a therapeutic agent known or believed to be capable of treating migraine, fibromyalgia, amyotrophic lateral sclerosis, irritable bowel syndrome, social phobia, Parkinson's disease, asthma, cough, or chronic obstructive pulmonary disease, respectively, at least one symptom of migraine, fibromyalgia, amyotrophic lateral sclerosis, irritable bowel syndrome, social phobia, Parkinson's disease, asthma, cough, or chronic obstructive pulmonary disease, respectively, or at least one condition associated with migraine, fibromyalgia, amyotrophic lateral sclerosis, irritable bowel syndrome, social phobia, Parkinson's disease, asthma, cough, or chronic obstructive pulmonary disease, respectively.

The amount of GABA analog prodrug of Formula (I), Formula (II), Formula (III), or Formula (IV) that will be effective in the treatment of migraine, fibromyalgia, amyotrophic lateral sclerosis, irritable bowel syndrome, social phobia, Parkinson's disease, asthma, cough, or chronic obstructive pulmonary disease in a patient will depend, in part, on the nature of the condition and can be determined by standard clinical techniques known in the art. In addition, in vitro or in vivo assays may be employed to help identify optimal dosage ranges. A therapeutically effective amount of prodrug of Formula (I), Formula (II), Formula (III), or Formula (IV) to be administered may also depend on, among other factors, the subject being treated, the weight of the subject, the severity and/or symptoms of the migraine, fibromyalgia, amyotrophic lateral sclerosis, irritable bowel syndrome, social phobia, Parkinson's disease, asthma, cough, or chronic obstructive pulmonary disease, the manner of administration and the judgment of the prescribing physician.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

In some embodiments, oral sustained release dosage forms are adapted to be administered to a patient from 1 to 3 times per day. In some embodiments, an oral sustained release dosage forms are adapted to be administered to a patient 1 or 2 times per day. Dosing can be provided alone or in combination with other drugs and may continue as long as required for effective treatment of migraine, fibromyalgia, amyotrophic lateral sclerosis, irritable bowel syndrome, social phobia, Parkinson's disease, asthma, cough, or chronic obstructive pulmonary disease.

Suitable dosage ranges for oral administration may depend on the potency of the particular GABA analog drug (once cleaved from the promoiety or promoieties) but may be from about 0.1 mg to about 200 mg of drug per kilogram body weight per day, for example, from about 1 to about 100 mg/kg-body weight per day. In certain embodiments, a compound of Formula (I) may be administered to a patient in an amount from about 10 mg-equivalents to about 3600 mg-equivalents of gabapentin per day, in certain embodiments, from about 200 mg-equivalents to about 2400 mg-equivalents of gabapentin per day, and in certain embodiments, from about 400 mg-equivalents to about 1600 mg-equivalents of gabapentin per day, to treat migraine, fibromyalgia, amyotrophic lateral sclerosis, irritable bowel syndrome, social phobia, Parkinson's disease, asthma, cough, or chronic obstructive pulmonary disease.

In certain embodiments, a compound of Formula (II) may be administered to a patient in an amount from about 10 mg-equivalents to about 1200 mg-equivalents of pregabalin per day, in certain embodiments, from about 50 mg-equivalents to about 800 mg-equivalents of pregabalin per day, and in certain embodiments, from about 100 mg-equivalents to about 600 mg-equivalents of pregabalin per day to treat migraine, fibromyalgia, amyotrophic lateral sclerosis, irritable bowel syndrome, social phobia, Parkinson's disease, asthma, cough, or chronic obstructive pulmonary disease. Dosage ranges may be determined by methods known to those skilled in the art.

In certain embodiments, a compound of Formula (III) may be administered to a patient in an amount from about 10 mg-equivalents to about 3600 mg-equivalents of gabapentin per day, in certain embodiments, from about 200 mg-equivalents to about 2400 mg-equivalents of gabapentin per day, and in certain embodiments, from about 400 mg-equivalents to about 1600 mg-equivalents of gabapentin per day, to treat migraine, fibromyalgia, amyotrophic lateral sclerosis, irritable bowel syndrome, social phobia, Parkinson's disease, asthma, cough, or chronic obstructive pulmonary disease.

In certain embodiments, a compound of Formula (IV) may be administered to a patient in an amount from about 10 mg-equivalents to about 1200 mg-equivalents of pregabalin per day, in certain embodiments, from about 50 mg-equivalents to about 800 mg-equivalents of pregabalin per day, and in certain embodiments, from about 100 mg-equivalents to about 600 mg-equivalents of pregabalin per day to treat migraine. Other GABA analogs may be more potent than gabapentin or pregabalin and lower doses may be appropriate for both the cleaved drug and any prodrug (measured on an equivalent molar basis). Dosage ranges may be determined by methods known to those skilled in the art.

A dose may be administered in a single dosage form or in multiple dosage forms. When multiple dosage forms are used the amount of compound contained within each dosage form may be the same or different. The amount of a compound of Formula (I), Formula (II), Formula (III), or Formula (IV) contained in a dose may depend on the route of administration and whether the migraine, fibromyalgia, amyotrophic lateral sclerosis, irritable bowel syndrome, social phobia, Parkinson's disease, asthma, cough, or chronic obstructive pulmonary disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. A dose of a pharmaceutical composition provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

The efficacy of administering at least one compound of Formula (I), Formula (II), Formula (III), or Formula (IV) for treating migraine can be assessed using animal and human models of migraine and clinical studies. Animal and human models of migraine are known (see, e.g., Iversen, Cephalalgia, 2001, 21, 781-785; Gras et al., Eur J Pharmacol 2000, 410, 43-51; Reuter et al., Funct Neurol 2000, 15 (Suppl), 9-18; Parsons and Bingham, Funct Neurol 2000, 15 (Suppl), 36-43; and De Vries et al., Eur J Pharamcol 1999, 375, 61-74). For example, to delineate and assess the effectiveness of a GABA analog prodrug provided by the present disclosure, the frequency of migraine attacks, their severity and their accompanying symptoms may be recorded and measured at baseline, and at 3 months, and 6 months, etc., following initiation of treatment.

Therapeutic activity of the pharmaceutical composition for treating migraine may be determined in various animal models of neuropathic pain or in clinically relevant studies of different types of neuropathic pain (see e.g., Eaton, J Rehabilitation Research and Development, 2003, 40(4), 41S-54S). The therapeutic activity may be determined without determining a specific mechanism of action. Animal models for neuropathic pain are known in the art and include, but are not limited to, animal models that determine analgesic activity or compounds that act on the CNS to reduce the phenomenon of central sensitization that results in pain from non-painful or non-noxious stimuli. Other animal models are known in the art, such as hot plate tests, model acute pain and are useful for determining analgesic properties of compounds that are effective when painful or noxious stimuli are present. The progression of migraine is believed to be similar to the progress of epilepsy because an episodic phenomenon underlies the initiation of the epileptic episode and, as such, it is believed that epilepsy animal models may be useful in determining a component of the therapeutic activity of the pharmaceutical composition. The therapeutic activity of the pharmaceutical composition may also be determined in animal models (see e.g., Gras et al., European J Pharmacology 2000, 410, 43-51; De Vries et al., European J Pharmacology 1999, 375, 61-74; and Reuter et al., Functional Neurology, 2000, 15(3), S9-S18) and human models (see e.g., Iverson, Cephalalgia, 2001, 21, 781-785) of migraine.

The efficacy of administering at least one compound of Formula (I), Formula (II), Formula (III), or Formula (IV) for treating fibromyalgia may be assessed using animal and human models of fibromyalgia and on clinical results (see e.g., Dooley et al., U.S. Application Publication No. 2004/0180959; Taylor et al., U.S. Application Publication No. 2004/0138305; Crofford et al., Arthritis & Rheumatism 2005, 52, 4, 1264-1273; Eaton, J Rehabilitation Research and Development 2003, 40(4), 41S-54S; Guay, Am J Geriatr Pharmacother, 2005, 3, 274-287; Freynhagen et al., Pain, 2005, 115, 254-263; Backonja et al., Clin Ther. 2003, 25, 81-104; Gidal et al., Am J Manag Care. 2006, 12, S269-S278; and Argoff, JAOA, 2002, Suppl. 3, 102(9), S21-S26). In particular, animal models of neuropathic pain or clinically relevant studies of different types of neuropathic pain have been found useful in assessing therapeutic activity for treating fibromyalgia.

The efficacy of administering at least one compound of Formula (I), Formula (II), Formula (III), or Formula (IV) for treating ALS may be assessed using animal and human models of ALS and clinical studies. Natural disease models of ALS include mouse models (motor neuron degeneration, progressive motor neuropathy, and wobbler) and the hereditary canine spinal muscular atrophy canine model (Pioro and Mitsumoto, Clin Neurosci, 1995-1996, 3(6), 375-85). Experimentally produced and genetically engineered animal models of ALS are also useful in assessing therapeutic efficacy (see e.g., Doble and Kennelu, Amyotroph Lateral Scler Other Motor Neuron Disord. 2000, 1(5), 301-12; Grieb, Folia Neuropathol. 2004, 42(4), 239-48; and Price et al., Rev Neurol (Paris), 1997, 153(8-9), 484-95). Specifically, the SOD 1-G93A mouse model is a recognized model for ALS. Examples of clinical trial protocols useful in assessing treatment of ALS are described, for example, in Mitsumoto, Amyotroph Lateral Scler Other Motor Neuron Disord. 2001, 2 Suppl 1, S 10-S 14, and Ludolph and Sperfeld, Neurodegener Dis. 2005, 2(3-4), 215-9.

The efficacy of administering at least one compound of Formula (I), Formula (II), Formula (III), or Formula (IV) for treating IBS may be assessed in clinical studies (see e.g., Lesbros-Pantoflickova et al., Aliment Pharmacol Ther. 2004, 20(11), 1253-1269; and Lee et al., Aliment Pharmacol Ther. 2005, 22(1), 981-88) and/or using animal models (see e.g., Mayer and Collins, Gastroenterology. 2002, 1222(7), 2032-48). An animal model of chronic visceral hypersensitivity using mechanical and chemical irritation of the colon of neonatal rats provides a convenient model for studying IBS, validating the neurogenic components of functional abdominal pain, and testing agents that may reduce visceral hypersensitivity (Al-Chaer et al., Gastroenterology 2000, 119, 1276-1285). For clinical studies, patients may be evaluated for IBS based on the Rome H criteria. Patients maintain a daily diary of their IBS symptoms, such as for example, pain relieved by defecation, stool frequency, stool consistency, abdominal distension, passage of mucus, and completion of evacuation, prior to and during treatment with a test compound. In addition, patients may be asked to globally assess their subjective improvement on a scale of 0-4.

The efficacy of administering at least one compound of Formula (I), Formula (II), Formula (III), or. Formula (IV) for treating social phobia may be assessed in clinical trials using criteria based on, for example, the Social Interaction Anxiety Scale (see, e.g., Rodebaugh et al., Psychol Assess 2006, 18(2), 231-7), the Liebowitz Social Anxiety Score (Stein et al., Psychopharmacology (Berl). 2001, 158(3), 267-72; Liebowitz et al., J Clin Psychiatry. 2002, 63(1), 66-74; and Stein et al., JAMA. 1998, 280(8), 708-13), and/or other social phobia assessment tools (Tharwani and Davidson, Psychiatr Clin North Am. 2001, 24(4), 643-59). Animal models of social anxiety disorders such as the subordination stress model, the variable-foraging-demand model, and animal attachment models, which may also be employed for assessing the efficacy of compounds provided by the present disclosure for treating social phobia and are described, for example, in Mathew et al., Am J Psychiatry. 2001, 158(1), 1558-1567.

The efficacy of administering at least one compound of Formula (I), Formula (II), Formula (III), or Formula (IV) for treating Parkinson's disease may be assessed using animal and human models of Parkinson's disease and clinical studies. Animal and human models of PD are known (see, e.g., O'Neil et al., CNS Drug Rev. 2005, 11(1), 77-96; Faulkner et al., Ann. Pharmacother. 2003, 37(2), 282-6; Olson et al., Am. J. Med. 1997, 102(1), 60-6; Van Blercom et al., Clin Neuropharmacol. 2004, 27(3), 124-8; Cho et al., Biochem. Biophys. Res. Commun. 2006, 341, 6-12; Emborg, J. Neuro. Meth. 2004, 139, 121-143; Tolwani et al., Lab Anim Sci 1999, 49(4), 363-71; Hirsch et al., J Neural Transm Suppl 2003, 65, 89-100; Orth and Tabrizi, Mov Disord 2003, 18(7), 729-37; and Betarbet et al., Bioessays 2002, 24(4), 308-18).

The efficacy of administering at least one compound of Formula (I), Formula (II), Formula (III), or Formula (IV) for treating cough may be assessed using animal models of cough and in clinical studies. For example, animal models of cough are described, for example, by Bolser et al., Br. J Pharmacol 1993, 110, 491-495; Adcock et al., Br. J Pharmacology 2003, 138, 407-416; Bolser et al., Eur J Pharmacol 1995, 277, 159-164; Lewis et al., Pulmonary Pharmacology & Therapeutics 2007, 20, 325-333; Mackenzie et al., Drug Discovery Today 2004, 1(3), 297-302; and Bolser et al., J Applied Physiology 1999, 86(3), 1017-1024. A capsaicin cough model in healthy human subjects is described, for example, in Dicpinigaitis and Dobkin, Chest 1997, 111(4), 996-999.

The efficacy of administering at least one compound of Formula (I), Formula (II), Formula (III), or Formula (IV) for treating asthma may be assessed using animal models of asthma and in clinical studies. For example, murine models of asthma are described by Kips et al., Eur Respir J 2003, 22, 374-382.

The efficacy of administering at least one compound of Formula (I), Formula (II), Formula (III), or Formula (IV) for treating chronic obstructive pulmonary disease may be assessed using animal models of chronic obstructive pulmonary disease and in clinical studies. For example, murine models of chronic obstructive pulmonary disease are described by Brusselle et al., Pulmonary Pharmacology & Therapeutics 2006, 19, 155-165; and Vlahos et al., Pulmonary Pharmacology & Therapeutics 2006, 19, 12-17; Kinoshita et al., Biochemical Biophysical Res Commun 2007, 354, 712-719; Churg et al., Am J Respir Crit Care Med 2003, 168, 199-207; and Mortorana et al., Am J Crit Care Med 2005, 172, 848-853.

In certain embodiments, oral administration of an oral sustained release dosage form comprising a compound of Formula (I), Formula (II), Formula (III), or Formula (IV) can provide a therapeutically effective concentration of a GABA analog such as gabapentin or pregabalin, in the blood plasma of a patient for a time period of at least about 4 hours after administration of the dosage form, in certain embodiments, for a time period of at least about 8 hours, and in certain embodiments, for a time period of at least about 12 hours, and in certain embodiments, for a time period of at least about 24 hours.

A GABA analog prodrug provided by the present disclosure or pharmaceutical composition thereof may be administered to a patient in need of treatment for migraine, fibromyalgia, amyotrophic lateral sclerosis, irritable bowel syndrome, social phobia, Parkinson's disease, asthma, cough, or chronic obstructive pulmonary disease in a therapeutically effective amount. A therapeutically effective amount refers to a total amount of GABA analog prodrug that results in a detectable change in the frequency or severity of the patient's migraine, fibromyalgia, amyotrophic lateral sclerosis, irritable bowel syndrome, social phobia, Parkinson's disease, asthma, cough, or chronic obstructive pulmonary disease symptoms. A therapeutically effective amount may provide a concentration of the GABA analog prodrug that is pharmacologically active and therapeutically effective.

### Combination Therapy

In certain embodiments, at least one GABA analog prodrug of Formula (I), Formula (II), Formula (III), or Formula (IV), or pharmaceutical compositions of any of the foregoing may be used in combination therapy with at least one other therapeutic agent including a different GABA analog prodrug of Formula (I), Formula (II), Formula (III), or Formula (IV). The GABA analog prodrug of Formula (I), Formula (II), Formula (III), or Formula (IV), or pharmaceutical composition of any of the foregoing and the additional therapeutic agent may act additively or, in certain embodiments, synergistically, such that the combination of the therapeutic agents together are, for example, more effective, safer, and/or produce fewer or less severe side effects. In certain embodiments, a GABA analog prodrug of Formula (I), Formula (II), Formula (III), or Formula (IV), or a pharmaceutical composition of any of the foregoing can be administered concurrently with the administration of another therapeutic agent. In certain embodiments, a GABA analog prodrug of Formula (I), Formula (II), Formula (III), or Formula (IV), or pharmaceutical composition of any of the foregoing can be administered prior or subsequent to administration of another therapeutic agent and thus can have regimens with overlapping schedules. The additional therapeutic agent may be effective for treating migraine, fibromyalgia, amyotrophic lateral sclerosis, irritable bowel syndrome, social phobia, Parkinson's disease, asthma, cough, or chronic obstructive pulmonary disease, may be effective in treating at least one symptom of migraine, fibromyalgia, amyotrophic lateral sclerosis, irritable bowel syndrome, social phobia, Parkinson's disease, asthma, cough, or chronic obstructive pulmonary disease, may be effective in treating a side effect of administering the GABA analog prodrug of Formula (I), Formula (II), Formula (III), or Formula (IV) for treating migraine, fibromyalgia, amyotrophic lateral sclerosis, irritable bowel syndrome, social phobia, Parkinson's disease, asthma, cough, or chronic obstructive pulmonary disease, or may be effective for treating a disease, disorder, or condition other than migraine, fibromyalgia, amyotrophic lateral sclerosis, irritable bowel syndrome, social phobia, Parkinson's disease, asthma, cough, or chronic obstructive pulmonary disease. In certain embodiments in which a compound of Formula (I), Formula (II), Formula (III), or Formula (IV) is administered together with an additional therapeutic agent for treating migraine, fibromyalgia, amyotrophic lateral sclerosis, irritable bowel syndrome, social phobia, Parkinson's disease, asthma, cough, or chronic obstructive pulmonary disease each of the active agents may be used at lower doses than when used singly.

Methods provided by the present disclosure include administration of one or more compounds or pharmaceutical compositions provided by the present disclosure and one or more other therapeutic agents provided that the combined administration does not inhibit the therapeutic efficacy of the one or more compounds provided by the present disclosure and/or does not produce adverse combination effects.

In certain embodiments, compositions provided by the present disclosure may be administered concurrently with the administration of another therapeutic agent, which may be part of the same pharmaceutical composition or dosage form, or in a different composition or dosage form than that containing the compounds provided by the present disclosure. In certain embodiments, compounds provided by the present disclosure may be administered prior or subsequent to administration of an additional therapeutic agent. In certain embodiments of combination therapy, the combination therapy comprises alternating between administering a composition provided by the present disclosure and a composition comprising an additional therapeutic agent, e.g., to minimize adverse side effects associated with a particular drug. When a compound provided by the present disclosure is administered concurrently with another therapeutic agent that potentially can produce adverse side effects including, but not limited to, toxicity, the therapeutic agent may advantageously be administered at a dose that falls below the threshold at which the adverse side effect is elicited.

The weight ratio of a compound provided by the present disclosure to a second therapeutic agent may be varied and may depend upon the effective dose of each agent. A therapeutically effective dose of each compound will be used. Thus, for example, when a compound provided by the present disclosure is combined with another therapeutic agent, the weight ratio of the compound provided by the present disclosure to the second therapeutic agent can be from about 1000:1 to about 1:1000, and in certain embodiments, from about 200:1 to about 1:200.

Combinations of a compound provided by the present disclosure and a second therapeutic agent may also be within the aforementioned range, but in each case, an effective dose of each active compound can be used. In such combinations a compound provided by the present disclosure and second therapeutic agent may be administered separately or in conjunction. In addition, the administration of one element may be prior to, concurrent with, or subsequent to the administration of another therapeutic agent(s). Accordingly, compounds of Formula (I), Formula (II), Formula (III), or Formula (IV) may be used alone or in combination with other therapeutic agents that are known to be beneficial in treating migraine, fibromyalgia, amyotrophic lateral sclerosis, irritable bowel syndrome, social phobia, Parkinson's disease, asthma, cough, or chronic obstructive pulmonary disease or other therapeutic agents that affect receptors or enzymes that either increase the efficacy, safety, convenience, or reduce unwanted side effects or toxicity of the compounds provided by the present disclosure. A compound of Formula (I), Formula (II), Formula (III), or Formula (IV) and the other therapeutic agent may be co-administered, either in concomitant therapy or in a fixed combination. The additional therapeutic agent may be administered by the same or different route than the route used to administer a compound of Formula (I), Formula (II), Formula (III), Formula (IV), or pharmaceutical composition of any of the foregoing.

In certain embodiments, GABA analog prodrugs provided by the present disclosure or pharmaceutical compositions thereof may be administered to a patient for the treatment of migraine in combination with a therapy or therapeutic agent known or believed to be effective in the treatment of migraine, or in certain embodiments, a disease, disorder, or condition associated with migraine. Drug therapy for migraine may be tailored to the severity and frequency of migraine headaches. For occasional attacks, acute treatment may be indicated, but for attacks occurring two or more times per month, or when attacks greatly impact the patient's daily life, prophylactic therapy on an ongoing basis may be indicated.

Drugs useful for treating migraine can prevent a migraine from occurring, abort a migraine that is beginning, or relieve pain during the migraine episode.

Prophylactic migraine treatments reduce the frequency of migraines and include non-steroidal anti-inflammatory agents (NSAIDs), adrenergic beta-blockers, calcium channel blockers, tricyclic antidepressants, selective serotonin reuptake inhibitors, anticonvulsants, NMDA receptor antagonists, angiotensin converting enzyme (ACE) inhibitors, angiotensin-receptor blockers (ARBs), leukotriene-antagonists, dopamine agonists, selective 5HT-1D agonists, selective 5HT-1F agonists, AMPA/KA antagonists, CGRP (calcitonin gene related peptide) antagonists, NOS (nitric oxide synthase) inhibitors, blockers of spreading cortical depression, and other therapy. Examples of NSAIDs useful for preventing migraine include aspirin, ibuprofen, fenoprofen, flurbiprofen, ketoprofen, mefenamic acid, and naproxen. Examples of adrenergic beta-blockers useful for preventing migraine include acebutolol, atenolol, imilol, metoprolol, nadolol, pindolol, propranolol, and timolol. Examples of calcium channel blockers useful for preventing migraine include amlodipine, diltiazem, dotarizine, felodipine, flunarizine, nicardipine, nifedipine, nimodipine, nisoldipine, and verapamil. Examples of tricyclic antidepressants useful for preventing migraine include amitriptyline, desipramine, doxepin, imipramine, nortriptyline, and protriptyline. Examples of selective serotonin reuptake inhibitors (SSRIs) useful for preventing migraine include fluoxetine, methysergide, nefazodone, paroxetine, sertraline, and venlafaxine. Examples of other antidepressants useful for preventing migraine include bupropion, nefazodone, norepinephrine, and trazodone.

Examples of anticonvulsants (antiepileptics) useful for preventing migraine include divalproex sodium, felbamate, gabapentin, lamotrigine, levetiracetam, oxcarbazepine, tiagabine, topiramate, valproate, and zonisamide. Examples of NMDA receptor antagonists useful for preventing migraine include dextromethorphan, magnesium, and ketamine. Examples of angiotensin converting enzyme (ACE) inhibitors useful for preventing migraine include lisinopril. Examples of angiotensin-receptor blockers (ARBs) useful for preventing migraine include candesartan. Examples of leukotriene-antagonists useful for preventing migraine include zileuton, zafirlukast, montelukast, and pranlukast. Examples of dopamine agonists useful for preventing migraine include α-dihydroergocryptine. Examples of other therapy useful for preventing migraine include botulinum toxin, magnesium, hormone therapy, riboflavin, methylergonovine, cyproheptadine, and phenelzine, and complementary therapies such as counseling/psychotherapy, relaxation training, progressive muscle relaxation, guided imagery, diaphragmatic breathing, biofeedback, acupuncture, and physical and massage therapy.

Acute migraine treatments intended to eliminate or reduce the severity of the headache and any associated symptoms after a migraine has begun include serotonin receptor agonists, such as triptans (5-hydroxytryptophan (5-HT) agonists) for example almotriptan, eletriptan, frovatriptan, naratriptan, rizatriptan, sumatriptan, and zolmitriptan; ergotamine-based compounds such as dihydroergotamine and ergotamine; antiemetics such as metoclopramide and prochlorperazine; and compounds that provide analgesic effects.

Other examples of drugs used to treat migraine once started include, acetaminophen-aspirin, caffeine, cyproheptadine, methysergide, valproic acid, NSAIDs such as diclofenac, flurbiprofen, ketaprofen, ketorolac, ibuprofen, indomethacin, meclofenamate, and naproxen sodium, opioids such as codeine, meperidine, and oxycodone, and glucocorticoids including dexamethasone, prednisone and methylprednisolone.

GABA analog prodrugs provided by the present disclosure may also be administered in conjunction with drugs that are useful for treating symptoms associated with migraine such as nausea and vomiting, and depression. Examples of useful therapeutic agents for treating or preventing vomiting include, but are not limited to, 5-HT₃ receptor antagonists such as ondansetron, dolasetron, granisetron, and tropisetron; dopamine receptor antagonists such as prochlorperazine, thiethylperazine, chlorpromazine, metoclopramide, and domperidone; glucocorticoids such as dexamethasone; and benzodiazepines such as lorazepam and alprazolam. Examples of useful therapeutic agents for treating or preventing depression include, but are not limited to, tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as fluoxetine, fluvoxamine, paroxetine, and setraline; monoamine oxidase inhibitors such as isocarboxazid, pargyline, phenizine, and tranylcypromine; and psychostimulants such as dextroamphetamine and methylphenidate.

In certain embodiments, GABA analog prodrugs provided by the present disclosure and pharmaceutical compositions thereof may be administered to a patient for treating fibromyalgia in combination with a therapy or another therapeutic agent known or believed to be effective in treating fibromyalgia, or in certain embodiments, a disease, disorder, or condition associated with fibromyalgia. Drug therapy for fibromyalgia may be tailored to the severity and frequency of fibromyalgia episodes. For occasional episodes, acute treatment may be indicated. For fibromyalgia episodes occurring two or more times per month, or when attacks greatly impact the patient's daily life, chronic therapy on an ongoing basis may be appropriate.

Treatments for fibromyalgia that reduce the frequency of episodes include non-steroidal anti-inflammatory agents (NSAIDs), adrenergic beta-blockers, calcium channel blockers, tricyclic antidepressants, selective serotonin reuptake inhibitors, anticonvulsants, NMDA receptor antagonists, dopamine agonists, selective 5-HT₃ receptor antagonists, opioids, muscle relaxants, sedative hypnotics, and other therapy. Examples of NSAIDs useful for treating fibromyalgia include aspirin, ibuprofen, fenoprofen, flurbiprofen, ketoprofen, mefenamic acid, and naproxen. Examples of adrenergic beta-blockers useful for treating fibromyalgia include acebutolol, atenolol, imilol, metoprolol, nadolol, pindolol, propranolol, and timolol. Examples of calcium channel blockers useful for treating fibromyalgia include amlodipine, diltiazem, dotarizine, felodipine, flunarizine, nicardipine, nifedipine, nimodipine, nisoldipine, and verapamil. Examples of tricyclic antidepressants useful for treating fibromyalgia include amitriptyline, desipramine, doxepin, imipramine, nortriptyline, cyclobenzaprine, and protriptyline. Examples of selective serotonin reuptake inhibitors useful for treating fibromyalgia include fluoxetine, methysergide, nefazodone, paroxetine, sertraline, citalopram, and venlafaxine. Examples of other antidepressants useful for treating fibromyalgia include bupropion, nefazodone, norepinephrine, venlafaxine, duloxetine, and trazodone. Examples of anticonvulsants (antiepileptics) useful for treating fibromyalgia include divalproex sodium, felbamate, gabapentin, lamotrigine, levetiracetam, oxcarbazepine, tiagabine, topiramate, valproate, and zonisamide. Examples of NMDA receptor antagonists useful for treating fibromyalgia include dextromethorphan, magnesium, and ketamine: Examples of dopamine agonists useful for treating fibromyalgia include α-dihydroergocryptine. Examples of opioids useful for preventing fibromyalgia are tramadol, oxycodone, and methadone. An example of a muscle relaxant useful for treating fibromyalgia is cyclobenzaprine. Examples of therapies useful for treating fibromyalgia include exercise, interferon, growth hormone, hormone therapy, diet low in animal fat and high in fiber, and complementary therapies such as counseling/psychotherapy, relaxation training, progressive muscle relaxation, guided imagery, diaphragmatic breathing, biofeedback, acupuncture, and physical and massage therapy.

Acute fibromyalgia treatments intended to eliminate or reduce the severity of muscular/skeletal pain and any associated symptoms include serotonin receptor agonists, such as triptans (5-hydroxytryptophan (5-HT) agonists), for example, almotriptan, eletriptan, frovatriptan, naratriptan, rizatriptan, sumatriptan, and zolmitriptan; ergotamine-based compounds such as dihydroergotamine and ergotamine; antiemetics such as metoclopramide and prochlorperazine; and compounds that provide analgesic effects.

Other examples of drugs useful for treating fibromyalgia include acetaminophen-aspirin, caffeine, cyproheptadine, methysergide, valproic acid, NSAIDs such as diclofenac, flurbiprofen, ketaprofen, ketorolac, ibuprofen, indomethacin, meclofenamate, and naproxen sodium, opioids such as codeine, meperidine, and oxycodone, and glucocorticoids such as dexamethasone, prednisone, and methylprednisolone.

GABA analog prodrugs provided by the present disclosure can also be administered in conjunction with drugs that are useful for treating symptoms associated with fibromyalgia such as migraine headache, and depression. Examples of therapeutic agents useful for treating migraine include beta-blockers such as atenolol, metoprolol, proranolol, timolol, and nadolol; NSAIDS such as fenoprofen, flurbiprofen, ketoprofen, and naproxen; calcium channel blockers such as verapamil, diltiazem, nicardipine, nifedipine, and nimodipine; anti-epilepsy medication such gabapentin, divalproex sodium, and topiramate; tricyclic antidepressants such as amitriptyline, doxepin, imipramine, nortriptlyine, protriptyline, and desipramine; serotonin reuptake inhibitors such as fluoxetine, sertraline, paroxetine, nefazodone, and venlafazine. Examples of therapeutic agents useful for treating depression include tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as fluoxetine, fluvoxamine, paroxetine, and setraline; monoamine oxidase inhibitors such as isocarboxazid, pargyline, phenizine, and tranylcypromine; and psychostimulants such as dextroamphetamine and methylphenidate.

In certain embodiments, GABA analog prodrugs provided by the present disclosure and pharmaceutical compositions thereof may be administered to a patient for treating amyotrophic lateral sclerosis in combination with a therapy or another therapeutic agent known or believed to be effective in treating ALS, or in certain embodiments, a disease, disorder, or condition associated with ALS. Examples of therapeutic agents useful for treating ALS include baclofen, neurotrophic factors, riluzole, tizanidine, benzodiazepines such as clonazepan and dantrolene.

In certain embodiments, GABA analog prodrugs provided by the present disclosure and pharmaceutical compositions thereof may be administered to a patient for treating irritable bowel syndrome in combination with a therapy or another therapeutic agent known or believed to be effective in treating IBS, or in certain embodiments, a disease, disorder, or condition associated with IBS.

Examples therapeutic agents useful for treating IBS include muscarine receptor antagonists such as pirenzapine, methoctramine, ipratropium, tiotropium, scopolamine, methscopolamine, homatropine, homatropine methylbromide, and methantheline; 5-HT₄ agonists such as tegaserod, prucalopride; 5-HT₄ antagonists such as alosetron; 5-HT₃ antagonist such as cilansetron; 5-HT₄ agonists/5-HT₃ antagonist such as renzapride; κ-opiod agonists such as fedotozine and asimadoline; CRF-1 antagonists; chloride channel activators such as lubiprostone; benzodiazepines such as dextofisopam; neurokinin modulators such as talnetant; antibiotics and probiotics; α-2-adrenergic agents; cholecystokinin receptor antagonists such as loxiglumide, dexloxiglumide; antispasmodics such as hyoscyamine, and dicyclomine, anti-diarrheals such as loperamide and diphenoxylate; fiber supplements such as psyllium, methylcellulose, and calcium polycarbophil; tricyclic antidepressants such as nortriptyline, desipamine, amitriptyline, and imipramine; SSRIs; antidepressants such as fluoxetine and paroxetine, buproprion, and venlafaxine.

Treatment of IBS may comprise psychological therapies. Treatment of IBS may also comprise modification of a patient's diet. For example, it may be recommended that an IBS patient avoid beans, cabbage, sorbitol, and fructose: A low-fat, high-fiber diet may also help some IBS patients. Regular physical activity may also help maintain proper functioning of the gastrointestinal tract.

In certain embodiments, GABA analog prodrugs provided by the present disclosure and pharmaceutical compositions thereof may be administered to a patient for treating social phobia in combination with a therapy or another therapeutic agent known or believed to be effective in treating social phobia, or in certain embodiments, a disease, disorder, or condition associated with social phobia.

Examples of therapeutic agents useful in treating social phobia include sertraline.

Therapeutic agents useful for treating diseases, disorders, and conditions associated with social phobia such as other anxiety disorders, for example, panic attack, agoraphobia, panic disorder, specific phobia, obsessive-compulsive disorder, posttraumatic stress disorder, acute stress disorder, generalized anxiety disorder, anxiety disorder due to a general medical condition, substance-induced anxiety disorder, include anti-depressant agent include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Examples of useful norepinephrine reuptake inhibitors include tertiary amine tricyclics, and secondary amine tricyclics. Examples of useful tertiary amine tricyclics include amitriptyline, clomipramine, doxepin, imipramine, and trimipramine. Examples of useful secondary amine tricyclics include amoxapine, desipramine, maprotiline, nortriptyline, and protriptyline. Examples of useful selective serotonin reuptake inhibitors include fluoxetine, fluvoxamine, paroxetine, citalopram, venlafaxine, mirtazapine, nefazodone, and sertraline. Examples of useful monoamine oxidase inhibitors include isocarboxazid, phenelzine, tranylcypromine, and selegiline. Examples of useful reversible inhibitors of monoamine oxidase include moclobemide. Examples of useful serotonin and noradrenaline reuptake inhibitors include venlafaxine. Examples of useful atypical anti-depressants include bupropion, lithium, nefazodone, trazodone, and viloxazine. Anti-anxiety agents include benzodiazepines and 5-HT_{IA} agonists or antagonists, such as 5-HT_{IA} partial agonists, and corticotropin releasing factor (CRF) antagonists. Examples of useful benzodiazepines include alprazolam, chlordiazepoxide, clonazepam, chlorazepate, diazepam, halazepam, lorazepam, oxazepam, brotizolam, clobazam, demoxepam, estazolam, flumazenil, flurazepam, midazolam, nitrazepam, nordazepam, quazepam, temazepam, triazolam, and prazepam. Examples of useful 5-HT_{IA} receptor agonists or antagonists include buspirone, flesinoxan, gepirone, tiospirone, zolpicone, zolpidem, zalepion, and ipsapirone. Other therapeutic agents useful for treating social phobia and associated diseases, disorders, and conditions include tranquilizers, such as barbituates, e.g., amobarbital, aprobarbital, butabarbital, butalbital, mephobarbital, methohexital, pentobarbital, phenobarbital, secobarbital, and thiopental; and propanediol carbamates, such as meprobamate and tybamate.

In certain embodiments, GABA analog prodrugs provided by the present disclosure and pharmaceutical compositions thereof may be administered to a patient for treating Parkinson's disease in combination with a therapy or another therapeutic agent known or believed to be effective in treating Parkinson's disease, or in certain embodiments, a disease, disorder, or condition associated with Parkinson's disease. Therapeutic agents useful for treating Parkinson's disease include dopamine precursors such levodopa, dopamine agonists such as bromocriptine, pergolide, pramipexole, and ropinirole, MAO-B inhibitors such as selegiline, anticholinergic drugs such as benztropine, trihexyphenidyl, tricyclic antidepressants such as amitriptyline, amoxapine, clomipramine, desipramine, doxepin, imipramine, maprotiline, nortriptyline, protriptyline, amantadine, and trimipramine, some antihistamines such as diphenhydramine; antiviral drugs such as amantadine; and beta blockers such as propranolol.

In certain embodiments, GABA analog prodrugs provided by the present disclosure and pharmaceutical compositions thereof may be administered to a patient for treating cough in combination with a therapy or another therapeutic agent known or believed to be effective in treating cough, or in certain embodiments, a disease, disorder, or condition associated with cough. Examples of drugs useful for treating cough include acetaminophen, benzonatate, carbetapentane, carbinoxamine, chlorpheniramine, codeine, dextromethorphan, diphehydramine, guaiacol sulfonate, guaifenesin, homatropine, homatropine methyl bromide, hydrocodone, hydromorphone, moguisteine, potassium iodide, promethazine, and pseudoephedrine. Other antitussive therapies include nocicpetin/orphanin, tachykinins, transient receptor potential vallinoid receptor-1 (TRPV-1) antagonists, postassium channel openers, diuretics, and methylxanthines.

In certain embodiments, GABA analog prodrugs provided by the present disclosure and pharmaceutical compositions thereof may be administered to a patient for treating asthma in combination with a therapy or another therapeutic agent known or believed to be effective in treating asthma, or in certain embodiments, a disease, disorder, or condition associated with asthma. Examples of drugs useful in treating asthma include albuterol, aminophylline, beclomethasone, bitolterol, budesonide, cromolyn, ephedrine, epinephrine, flunisolide, fluticasone, formoterol, hydrocortisone, isoproterenol, levalbuterol, methylprednisolone, prednisolone, prednisone, pirbuterol, metaproterenol, racepinephrine, omalizumab, oxytriphylline, mometusone, montelukast, nedocromil, oxtriphylline, pirbuterol, salmeterol, terbutaline, theophylline,triamcinolone, zafirlukast, and zileuton.

In certain embodiments, GABA analog prodrugs provided by the present disclosure and pharmaceutical compositions thereof may be administered to a patient for treating chronic obstructive pulmonary disease in combination with a therapy or another therapeutic agent known or believed to be effective in treating chronic obstructive pulmonary disease, or in certain embodiments, a disease, disorder, or condition associated with chronic obstructive pulmonary disease. Examples of drugs useful for treating chronic obstructive pulmonary disease include albuterol, arformoterol, azithromycin, bitolterol, epinephrine, fluticasone, formoterol, ipratropium, isoproterenol, levabuterol, metaproterenol, pirbuterol, racepinephrine, salmeterol, and tiotropium. Useful drugs for treating chronic obstructive pulmonary disease include bronchodialators such as β2 agonists such as salbutamol, bambuterol, clenbuterol, fenoterol, and formoterol; M3 antimuscarinics such as ipratropium; leukotriene antagonists such as montelukast, pranlukast, and zafirlukast; cromones such as cromoglicate and nedocromil; xanthines such as theophylline; corticosteroids such as beclomethasone, mometasone, and fluticasone; and TNF antagonists such as infliximab, adalimumab, and etanercept. Other treatments for chronic obstructive pulmonary disease include oxygen therapy, and pulmonary rehabilitation.

### EXAMPLES

The invention is further defined by reference to the following examples, which describe synthesis of GABA analog prodrugs of Formula (I), Formula (II), Formula (III), and Formula (IV), preparation of sustained release dosage forms comprising at least one GABA analog prodrug, and methods of treating migraine fibromyalgia, amyotrophic lateral sclerosis, irritable bowel syndrome, social phobia, Parkinson's disease, asthma, cough, or chronic obstructive pulmonary disease comprising administering at least one GABA analog prodrug of Formula (I), Formula (II), Formula (III), or Formula (IV). It will be apparent to those skilled in the art that many modifications, both to materials and methods, may be practiced without departing from the scope of the invention. Examples 8-13 are prophetic.

In the examples below, the following abbreviations have the following meanings. If an abbreviation is not defined, the generally accepted meaning applies.
cm=centimeter
g=gram
h=hour
J=Joules
kp=kilopascal
kg=kilogram
kV=kilovolt
L=liter
LC/MS=liquid chromatography/mass spectroscopy
mA=milliamps

mg=milligram

min=minute

mol=moles

mL=milliliter

mm=millimeter

μg=microgram

μL=microliter

μm=micromolar

v/v=volume to volume

### Example 1

### 1-{[(α-Isobutanoyloxyethoxy)carbonyl]aminomethyl}-1-Cyclohexane Acetic Acid via a Trimethylsilyl Ester Intermediate

### Step A: 1-{[(α-Chloroethoxy)carbonyl]aminomethyl}-1-Cyclohexane Acetic Acid

In a 5-liter, 3-neck, round bottom flask containing dichloromethane (1.6 L) was added gabapentin (120.4 g, 0.704 mol) followed by triethylamine (294 mL, 2.11 mol). Chlorotrimethylsilane (178 mL, 1.40 mol) was slowly added while maintaining the reaction temperature below 15° C. and the resulting suspension was stirred for 30 min. 1-Chloroethyl chloroformate (100 g, 0.704 mol) was then slowly added while maintaining the temperature below 15° C. After the addition was complete, additional triethylamine (88 mL, 0.63 mol) was added and the resulting suspension was stirred at room temperature for 30 min. The resulting silyl ester was converted via acidic work-up to the corresponding acid by washing the reaction mixture with water (2×1 L), followed by 1N HCl (2×2 L), and then brine (2×500 mL). After drying over anhydrous sodium sulfate and removing the solvent in vacuo, the crude product (190 g) was obtained as an orange oil and used in Step B without further purification. ¹H NMR (CDCl₃, 400 MHz): δ 1.41-1.57 (m, 10H), 1.78 (d, 3H), 2.33 (s, 2H), 3.27 (d, 2H), 5.42 (br. s, 1H), 6.55 (q, 1H).

### Step B: 1-{[(α-Isobutanoyloxyethoxy)carbonyl]aminomethyl}-1-Cyclohexane Acetic Acid (3)

To a 3-liter, 3-neck, round bottom flask was added isobutyric acid (254 g, 2.9 mol) followed by triethylamine (395 mL, 2.84 mol). The reaction mixture was cooled to room temperature and a solution of crude acid from the above reaction step (190 g, 0.69 mol) in dichloromethane (80 mL) was added in a controlled fashion while maintaining the temperature below 30° C. The resulting pale yellow solution was stirred overnight. The reaction mixture was then diluted with one volume of dichloromethane and washed with water (6×500 mL), aqueous potassium bicarbonate (3×500 mL), and brine (2×500 mL). After drying over anhydrous sodium sulfate, removal of the solvent in vacuo afforded the crude product as a dark-red oil (87 g). A portion (35 g) of this product was loaded onto an 800 g Biotage™ normal phase silica gel flash column and eluted with 40% diethyl ether in hexane (6 L), which after removing the solvent in vacuo afforded the product as a colorless oil (13.5 g). This was repeated with a second 35 g portion of crude product yielding a further 13.5 g of 1-{[(α-isobutanoyloxyethoxy)carbonyl]aminomethyl}-1-cyclohexane acetic acid. A sample of the product (25 g) was recrystallized by dissolution in heptane (325 mL) at 70° C., followed by slow cooling to room temperature. The white crystalline product (23 g) was isolated by filtration. Melting point: 63-64° C.

### Example 2

### 1-{[(α-Isobutanoyloxyethoxy)carbonyl]aminomethyl}-1-Cyclohexane Acetic Acid via an Allyl Ester Intermediate

### Step A: Allyl 1-Aminomethyl-1-Cyclohexane Acetate Hydrochloride

A dry 3 L, three-neck, round-bottomed flash fitted with a magnetic stirring bar and a 500 mL pressure-equalizing addition funnel was flushed with nitrogen gas. The flask was charged with gabapentin (171 g, 1.0 mol) and ally! alcohol (1 L, 852 g, 14.6 mol) and the entire mixture was cooled to 0° C. in an ice-water bath. Thionyl chloride (225 mL, 360 g, 3.0 mol) was added dropwise over a period of 1 h to the stirred solution. The reaction mixture was stirred at room temperature for 16 h, and then was diluted with ethyl ether (2 L) and cooled to 0° C. while stirring. After several minutes white crystals formed, which were collected by filtration. The crude product was recrystallized from a 1/3 (v/v) mixture of ethanol and ethyl ether (2 L) to give the product as a white solid (220 g, 88% yield). m.p.: 138-142° C. ¹H NMR (CD₃OD, 400 MHz): δ 1.36-1.54 (m, 10H), 2.57 (s, 2H), 3.05 (s, 2H), 4.61 (d, J=6 Hz, 2H), 5.22 (dd, J=10.4, 1.2 Hz, 1H), 5.33 (dd, J=17.2, 1.4 Hz, 1H), 5.90-6.00 (m, 1H). MS (ESI) m/z 212.0 (M+Cl)⁺.

### Step B: Allyl 1-{[(α-Chloroethoxy)carbonyl]aminomethyl}-1-Cyclohexane Acetate

To a solution of the above hydrochloride salt (220 g, 0.89 mol) in dichloromethane (1 L) was slowly added 1-chloroethyl chloroformate (101.7 mL, 132.3 g, 0.92 mol). The reaction mixture was cooled to 0° C. and 4-methylmorpholine (205 mL, 188.9 g, 1.87 mol) slowly added over a period of 1 h while maintaining a temperature of less than 10° C. The resulting turbid solution was stirred at room temperature for 1 h. Ethanol (150 mL) was added and the reaction mixture was stirred at room temperature for 1 h. The reaction mixture was then diluted with ether (2.5 L), washed with water (1 L), and brine (1 L). The organic phase was dried over sodium sulfate and concentrated to give the title compound as a light yellow, viscous liquid (282 g, 100% yield). ¹H NMR (CDCl₃, 400 MHz): δ 1.35-1.58 (m, 10H), 1.78 (d, J=5.6 Hz, 3H), 2.32 (s, 2H), 3.22 (d, J=6.8 Hz, 2H), 4.57 (d, J=5.6 Hz, 2H), 5.25 (dd, J=10.4, 1 Hz, 1H), 5.32 (dd, J=17.2, 1.6 Hz, 1H), 5.52 (br, 1H, NH), 5.90-5.94 (m, 1H), 6.54 (q, J=5.6 Hz, 1H).

### Step C: Allyl 1-{[(α-Isobutanoyloxyethoxy)carbonyl]aminomethyl}-1-Cyclohexane Acetate

To a mixture of isobutyric acid (432 mL, 391.5 g, 4.4 mol) and 4-methylmorpholine (488 mL, 449 g, 4.4 mol) was added a solution of the chlorocarbamate from the previous step (282 g, 0.88 mol) in isobutyric acid (432 mL, 391.5 g, 4.4 mol). The addition occurred at 0° C. over a period of 30 min. The resulting turbid solution was stirred at room temperature for 16 h. The reaction mixture was diluted with ether (2.5 L) and washed with water (3×500 mL) followed by 10% aqueous potassium bicarbonate (6×500 mL), and then brine (500 mL). The organic phase was dried over sodium sulfate and concentrated to provide the title compound as a viscous liquid (328 g, 100% yield). ¹H NMR (CDCl₃, 400 MHz): δ 1.15 (d, J=7.2 Hz, 6H), 1.35-1.58 (m, 10H), 2.31 (s, 2H), 2.51 (m, 1H), 3.19 (d, J=5.6 Hz, 2H), 4.56 (d, J=5.6 Hz, 2H), 5.24 (dd, J=10, 1 Hz, 1H), 5.32 (dd, J=17, 1.2 Hz, 1H), 5.35 (br, 1H), 5.84-5.94 (m, 1H), 6.78 (q, J=5.6 Hz, 1H). MS (ESI) m/z 392.24 (M+H)⁺.

### Step D: Deprotection of Allyl 1-{[(α-Isobutanoyloxyethoxy)carbonyl]-aminomethyl}-1-Cyclohexane Acetate

To a stirred suspension of ammonium formate (112 g, 1.7 mol) in ethanol (500 mL) was added the above ally! ester (328 g, 0.88 mol) together with 10% Pd/C (15 g) under a nitrogen atmosphere. After 6 h, the reaction mixture was worked-up by filtering off the catalyst. The catalyst was washed with ethanol (2×250 mL) and the filtrates were combined and evaporated. The crude product was dissolved in ether (2 L) and the organic phase was washed with 2N HCl (2×2 L) to convert the ammonium salt into the acid form, followed by washing with water (1 L) and brine (1 L). The ether layer was dried over sodium sulfate and concentrated to give the crude product as a viscous liquid (240 g, 82% yield).

### Step E: Crystallization of 1-{[(α-Isobutanoyloxyethoxy)carbonyl]-aminomethyl}-1-Cyclohexane Acetic Acid

A 3 L round-bottom flask was equipped with a heating oil bath, a nitrogen inlet adapter, an internal thermometer, an overhead mechanical stirrer, and a reflux condenser. The flask was flushed with nitrogen and charged with a 1/10 (v/v) mixture of ethyl acetate/heptane (1.2 L) and the crude product from the preceding reaction (240 g). The flask was heated until the product dissolved, and then cooled according to the following schedule:

| Time | Internal | | |
|---|---|---|---|
| (min) | Temp (° C.) | Appearance | Remarks |
| 0 | 18 | Solid in solvent | Started heating oil |
| | | | bath |
| 10 | 48 | Turbid | Slow dissolution of |
| | | | product |
| 20 | 58 | Clear solution | Turn off oil bath |
| 25 | 60 | Clear solution | Maximum temp. |
| | | | reached |
| 45 | 43 | Turbid | Compound |
| | | | crystallizing |
| 60 | 36 | Milky solution | Seeded with pure ref. |
| | | | material |
| 90 | 24 | Solid in solution | — |

The flask was then cooled to 4° C. and stirred overnight (cooling improves the yield). The product was filtered and washed with heptane (2×100 mL), then dried under reduced pressure (25 mm of Hg (0.033 atm)) at 30° C. for 18 h to yield 1-{[(α-isobutanoyloxyethoxy)carbonyl]aminomethyl}-1-cyclohexane acetic acid (185 g) as a white crystalline solid.

### Example 3

### X-Ray Powder Diffraction Analysis of Crystalline 1-{[(α-Isobutanoyloxyethoxy)carbonyl]aminomethyl}-l-Cyclohexane Acetic Acid

X-ray powder diffractograms (XRPDs) of crystalline samples of 1-{[(α-isobutanoyloxyethoxy)carbonyl]aminomethyl}-1-cyclohexane acetic acid produced according to Examples 1 and 2 above were obtained using a Bruker D8 Discover X-ray powder diffractometer using Cu Kα radiation. The instrument was equipped with parallel beam optics and a two-dimensional HI-STAR area detector. The tube voltage and amperage were set to 40 kV and 40 mA, respectively. The collimated X-ray beam was reduced to a spot size of about 0.5 mm in diameter. The area detector was placed 15 cm from the center of the goniometer and the angular resolution is approximately 0.033°/pixel. The detector covered a range of 35° in 2-theta (2θ) within one frame. The angle between the X-ray beam and the horizontal sample plate was set to 4° and the center of the area detector was set to an angle of 18°. This geometry allowed the measurement of 2-theta from 4.5° to 39.5° within one frame. The typical averaging time was 3 minutes for each XRPD pattern collected. A corundum sample (NIST 1976) was used to calibrate the XRPD instrument. Both samples gave equivalent diffractogram patterns.

### Example 4

### Melting Point and Differential Scanning Calorimetry Analysis of Crystalline 1-{[(α-Isobutanoyloxyethoxy)carbonyl]aminomethyl}-1-Cyclohexane Acetic Acid

Melting points of crystalline samples of 1-{[(α-isobutanoyloxyethoxy)carbonyl]aminomethyl}-1-cyclohexane acetic acid produced according to Examples 1 and 2 above were measured using an Electrothermal 9200 melting point apparatus and determined to be 63-64° C.

Differential scanning calorimetry (DSC) analysis of crystalline samples of 1-{[(α-isobutanoyloxyethoxy)carbonyl]aminomethyl}-1-cyclohexane acetic acid produced according to Examples 1 and 2 above were measured using a Perkin Elmer Series 7 instrument, scanning from 25° C. to 250° C. at a scan rate of 5° C/min. A test portion of the sample was placed in an aluminum pan and the cap crimped to eliminate any visible seam between the cap and the pan. An empty pan was prepared in the same manner as a blank. The pans were placed in the Differential Scanning calorimeter. The samples were analyzed using an appropriate temperature gradient (Equilibration at Initial Temp, Isothermal, Ramp Rate, Final Temp). DSC analysis showed an endothermic transition with an onset temperature of 58.3° C. and a ΔH of 72.39 J/g. At the peak endotherm of 63-64° C. the sample visibly melted.

### Example 5

### {[1(1-Isobutanoyloxyethoxy)carbonyl]aminomethyl}-1-Cyclohexane Acetic Acid (3)

To a solution of gabapentin (6.8 g, 0.04 mol) in water (40 mL) was added a solution of [(1-isobutanoyloxyethoxy)carbonyloxy]succinimide (10 g, 0.036 mol) in acetonitrile (40 mL) over a period of 30 min. The reaction was stirred at ambient temperature for 3 hours. The reaction mixture was diluted with methyl tert-butyl ether (200 mL), washed with water (2×100 mL), and brine (50 mL). The organic phase was separated, dried over anhydrous sodium sulfate, filtered, and concentrated in vacuo to afford the title compound as a white solid (12 g, quantitative).

The following procedure was used to crystallize the title compound. The solid compound (12 g) was suspended in methylcyclohexane: methyl tert-butyl ether 10:1 (60 mL). The suspension was slowly heated up to 50° C. over a period of 30 min. The clear solution was then allowed to cool to room temperature. The turbid mixture was seeded with 5 mg of the title compound in crystalline form. The mixture was further cooled to 0-4° C. for 2 h. The solid product was filtered and washed with methylcyclohexane (2×10 mL) to provide the title compound (3) as a white crystalline solid (10 g, 83% yield). The crystalline solid material had a melting point of about 64-66° C. as determining using the open capillary melting point method.

### Example 6

### Preparation of a Sustained Release Oral Dosage Form of 1-{[(α-Isobutanoyloxyethoxy)carbonyl]aminomethyl}-Cyclohexane Acetic Acid (3)

Sustained release oral dosage forms containing the gabapentin prodrug, 1-{[(α-isobutanoyloxyethoxy)carbonyl]aminomethyl}-1-cyclohexane acetic acid (compound (3)), were prepared according the procedure disclosed in Cundy, U.S. Application Publication No. 2006/0141034, which is incorporated by reference herein in its entirety. Oral sustained release tablets containing compound (3) were made having the ingredients shown in Table 1:

TABLE 1
Ingredients of Oral Sustained Release Tablets

| | | Amount/ | Compo- | Ingredient |
|---|---|---|---|---|
| | | Tablet | sition | |
| Ingredient | Manufacturer | (mg/tablet) | (wt %) | Category |
| Compound | XenoPort | 600.00 | 45.80 | Prodrug |
| (3) | (Santa Clara, CA) | | | |
| Dibasic | Rhodia | 518.26 | 39.56 | Diluent |
| Calcium | (Chicago, IL) | | | |
| Phosphate, | | | | |
| USP | | | | |
| Glyceryl | Gattefosse | 60.05 | 4.58 | Lubricant/ |
| Behenate, NF | (Saint Pirest, | | | Release |
| | Cedex, France) | | | controlling |
| | | | | agent |
| Talc, USP | Barrett Minerals | 80.02 | 6.11 | Anti- |
| | (Mount Vernon, IN) | | | adherent |
| Colloidal | Cabot | 5.43 | 0.41 | Glidant |
| Silicon | (Tuscola, IL) | | | |
| Dioxide, NF | | | | |
| Sodium | Fisher | 24.00 | 1.84 | Surfactant |
| Lauryl | (Fairlawn, NJ) | | | |
| Sulfate, NF | | | | |
| Magnesium | Mallinckrodt | 22.22 | 1.69 | Lubricant |
| Stearate, NF | (Phillipsburg, NJ) | | | |
| Total | | 1310.00 | 100 | |

The tablets were made according to the following steps. Compound (3), dibasic calcium phosphate, glyceryl behenate, talc, and colloidal silicon dioxide were weighed out, screened through a #20 mesh screen and mixed in a V-blender for 15 minutes. The slugging portion of the sodium lauryl sulfate was weighed and passed through a #30 mesh screen. The slugging portion of the magnesium stearate was weighed and passed through a #40 mesh screen. Screened sodium lauryl sulfate and magnesium stearate were added to the V-blender and blended for 5 min. The blend was discharged and compressed into slugs of approximately 400 mg weight on a tablet compression machine. The slugs were then passed through a Comil 194 Ultra mill (Quadro Engineering, Inc., Millburn, N.J.) to obtain the milled material for further compression. The tableting portion of the sodium lauryl sulfate was weighed and passed through a #30 mesh screen. The tableting portion of the magnesium stearate was weighed and passed through a #40 mesh screen. The milled material and the tableting portions of the sodium lauryl sulfate and magnesium stearate were added to the V-blender and blended for 3 min. The blended material was discharged and compressed to form tablets having a total weight of about 1310 mg and a compound (3) loading of about 600 mg (45.8 wt %). The tablets had a mean final hardness of 16.1 to 22.2 kp (158 to 218 Newtons). It will be appreciated that the sustained release oral dosage form may optionally be coated. For example, a tablet may be coated with Opadry II (39.3 mg/tablet).

### Example 7

### Pharmacokinetics of Orally Administered 1-{[(α-Isobutanoyloxyethoxy)carbonyl]aminomethyl}-1-Cyclohexane Acetic Acid (3)

A randomized, crossover, fed/fasted single-dose study of the safety, tolerability, and pharmacokinetics of oral administration of 1-{[(α-isobutanoyloxyethoxy)carbonyl]aminomethyl}-1-cyclohexane acetic acid (3) in healthy adult subjects was conducted. The oral sustained release dosage form of Example 6 (uncoated) was used in this study. The study was designed to evaluate the performance of this formulation in humans in comparison with the commercial gabapentin capsule formulation (Neurontin®, Pfizer). Twelve healthy adult volunteers (7 males and 5 females) participated in the study. Mean body weight was 75.6 kg. All subjects received two different treatments in a random order with a one-week washout between treatments. The two treatments were: A) a single oral dose of Example 6 tablets (2×600 mg) after an overnight fast; and B) a single oral dose of Example 6 tablets (2×600 mg) after a high fat breakfast.

Blood and plasma samples were collected from all subjects prior to dosing, and at 0.5, 1, 1.5, 2, 3, 4, 6, 8, 12, 18, 24, and 36 hours after dosing. Urine samples were collected from all subjects prior to dosing, and complete urine output was obtained at the 0-4 h, 4-8 h, 8-12 h, 12-18 h, 18-24 h, and 24-36 h intervals after dosing. Blood samples were quenched immediately with methanol and stored frozen at ≦70° C. Sample aliquots were prepared for analysis of gabapentin and compound (3) using sensitive and specific LC/MS/MS methods.

The mean±SD Cₘₐₓ for gabapentin in blood after oral dosing of the tablets (fasted) was 4.21±1.15 μg/mL. Following administration of the tablets after a high fat breakfast, the Cₘₐₓ of gabapentin in blood was further increased to 6.24±1.55 μg/mL. The mean±SD AUC for gabapentin in blood after oral dosing of the tablets (fasted) was 54.5±12.2 μg·h/mL. Following administration of the tablets ’ after a high fat breakfast, the AUC of gabapentin in blood was further increased to 83.0±21.8 μg·h/mL. In the presence of food, exposure to gabapentin after oral administration of the tablets increased an additional 52% compared to that in fasted subjects.

The time to peak blood levels (Tₘₐₓ) of gabapentin was significantly delayed after oral administration of the tablets. In fasted subjects, oral administration of the tablets gave a gabapentin Tₘₐₓ of 5.08±1.62 h. This compares to a typical Tₘₐₓ of immediate release gabapentin of about 2-4 h. The gabapentin Tₘₐₓ after oral administration of the tablets was further delayed to 8.40±2.07 h in the presence of food. The apparent terminal elimination half-life for gabapentin in blood was similar for all treatments: 6.47±0.77 h for the tablets in fasted subjects, and 5.38±0.80 h for the tablets in fed subjects.

Following oral administration of the tablets, the percent of the gabapentin dose recovered in urine was 46.5±15.8% for fasted subjects and 73.7±7.2% for fed subjects.

Exposure to intact prodrug in blood after oral administration of the tablets was low. After oral dosing of the tablets in fasted subjects, concentrations of intact compound (3) in blood reached a maximum of 0.040 μg/mL, approximately 1.0% of the corresponding peak gabapentin concentration. Similarly, the AUC of compound (3) in blood of these subjects was 0.3% of the corresponding AUC of gabapentin in blood. After oral dosing of the tablets in fed subjects, concentrations of intact compound (3) in blood reached a maximum of 0.018 μg/mL, approximately 0.3% of the corresponding peak gabapentin concentration. Similarly, the AUC of compound (3) in blood of these subjects was less than 0.1% of the corresponding AUC of gabapentin in blood.

### Example 8

### Use of Animal Models and Clinical Trials to Assess the Efficacy of Compounds of Formulae (I)-(IV) for Treating Migraine

Therapeutic activity of compounds provided by the present disclosure may be determined in various animal models of neuropathic pain or in clinically relevant studies of different types of neuropathic pain. Animal models for neuropathic pain are known in the art and include, but are not limited to, animal models that determine analgesic activity or compounds that act on the CNS to reduce the phenomenon of central sensitization that results in pain from nonpainful or nonnoxious stimuli. Other animal models that are known in the art, such as hot plate tests, model acute pain and are useful for determining analgesic properties of compounds that are effective when painful or noxious stimuli are present. The progression of migraines is believed to be similar to the progression of epilepsy (because an episodic phenomenon underlies the initiation of the epileptic episode) and, as such, it is believed that epilepsy animal models may be useful in determining efficacy in treating migraine.

### Analgesic Activity

The following test may be used to evaluate the analgesic activity of a GABA analog prodrug. Test compound is administered orally to mice. Morphine is administered as a reference substance at 64 mg/kg to mice under the same experimental conditions. A vehicle is administered to mice as a control substance under the same experimental conditions. Test compound, morphine, or vehicle is administered to the mice in a blinded study. Sixty minutes after the test compound, morphine, or vehicle are administered, the mice are placed onto a hot metal plate maintained at 54° C. and surrounded by a Plexiglass cylinder (see e.g., Eddy et al., J. Pharmacol. Exp. Ther. 1953, 107, 385-393). The time taken for the mice to lick their feet is an index of analgesic activity. Effective analgesics increase the latency or amount of time to licking. Latency to the first foot lick is measured, up to a maximum time of 30 sec to prevent tissue damage to the mice.

### Hyperreflexia and Flexor Reflex Tests

Assessment of hyperreflexia, pain, and muscle tone in chronic spinally transected rats is performed using male albino Holtzman-derived rats weighing 270-530 gm. The rats are housed independently and have continuous access to food and water throughout the experiments. Animals are anesthetized using a mixture of isoflurane and oxygen at a flow rate of 4 L/min.

Rats are placed in a stereotaxic frame and anesthesia is maintained. An incision is made so that the paraspinal muscles can be retracted and a laminectomy, performed between T6-T9. A one- to two-millimeter portion of the spinal cord is removed by evacuation and replaced with gel foam to reduce bleeding, after which the incision is closed in layers.

Following the transection, rats are placed in a room in which the ambient temperature is raised to about 80° F. with a space heater to maintain body temperature. On the following morning post-surgery, the hindquarters of the spinalized rats are bathed and their urine expressed manually by applying pressure to their bladders. Experiments are conducted between 21 and 28 days after surgery. For the first two weeks post-surgery, 0.25 mL of an antibiotic is administered to the rats to prevent bladder infection. A topical antibiotic is applied to any part of the skin that shows signs of decubitus lesions. Within approximately two weeks, all animals regain bladder control and are no longer given antibiotic treatment. Assessment of hyperreflexia and flexor reflex is performed before and after drug treatment so that each animal serves as its own control.

Initial assessment of hyperreflexia is performed by rating the hyperreflexia response elicited with an innocuous stimulus, such as a metal probe. A metal probe is pressed against the lower abdomen at four specific sites. The response is evaluated for each of four trials using a scale ranging from zero (no response in all four trials) to four (a maximum, tonic-clonic reaction elicited in all four trials). All scores, pre- and post-treatment, are transformed to indicate the percent of hyperreflexia, pain, or muscle tone. The data is analyzed using appropriate statistical methods.

After determining hyperreflexia before drug treatment, test compound is administered to the rats.

Polysynaptic flexor-reflex responses, elicited by stimuli that activate high-threshold afferents, are recorded as EMG activity from the ipsilateral hamstring muscle. Supramaximal electric shocks are applied to the hindpaw and recording electrodes are placed in the biceps femoris semitendinosus muscle. Five sets of stimuli are made at each time point. The flexor reflex is recorded, in periods with and without test compound, every 30 min once a stable baseline response is achieved. The data at time zero represent pre-treatment control values. The responses are determined in spinalized rats by observing the flexor-reflex response before treatment and at each of 30, 60, 90, and 120 min following administration of test compound, baclofen (10 mg/kg s.c.) and vehicle (water, 12 ml/kg p.o.), respectively. Efficacy is indicated when a test compound is shown to reduce the magnitude of the flexor-reflex responses in a chronic spinalized rat at all time points with similar efficacy to baclofen, the positive control.

### Cutaneous Hypersensitivity Tests

The effects of test compound on nociceptive activation of the trigeminovascular system is determined using the migraine model described in Goadsby et al., Brain 2002, 125, 1392-1401. A pharmaceutical composition comprising a test compound is administered to cats. To serve as positive and negative controls, a vehicle control is administered to the cats. Efficacy is indicated for compounds that inhibit trigeminovascular activation compared to the trigeminovascular activation in the cats that receive the vehicle.

### Yawning

Yawning is a behavior that has been linked to activation of dopaminergic neurotransmission. Yawning is part of a behavioral syndrome occurring in most patients during a migraine attack. Blockage of quinpirole-induced yawning in rats has been used as an animal model to study the potential antagonism of migraine symptoms.

Male Sprague Dawley rats are acclimatized for 12 days before testing and at the time of the study. The rats are housed in standard size steel cages with four animals per cage and are maintained on a 12 hour light/dark schedule.

Test compound or vehicle is administered 15 min before the dopamine D2 agonist quinpirole in vehicle or the vehicle alone is administered to the animals. The animals are then placed individually in a 6 in×6 in plexiglass observation cages and the number of yawns is counted for the subsequent 30 min. The data is analyzed by an appropriate statistical method.

The dopamine D2 agonist quinpirole can produce an average of 13-15 yawns per 30 minutes while no yawning behavior is typically observed in vehicle treated animals. Compounds that inhibit quinpirole-induced yawning may be efficacious in treating migraine.

### Animal Model of Dural Protein Extravasation

The following animal model can be employed to determine the ability of a GABA analog prodrug to inhibit protein extravasation, an exemplary functional assay of the neuronal mechanism of migraine.

Rats or guinea pigs are anesthetized with sodium pentobarbital intraperitoneally (65 mg/kg or 45 mg/kg respectively) and placed in a stereotaxic frame with the incisor bar set at −3.5 mm for rats or −4.0 mm for guinea pigs. Following a midline sagital scalp incision, two pairs of bilateral holes are drilled through the skull (6 mm posterially, 2.0 and 4.0 mm laterally in rats; 4 mm posteriorly and 3.2 and 5.2 mm laterally in guinea pigs, all coordinates referenced to bregma). Pairs of stainless steel stimulating electrodes, insulated except at the tips are lowered through the holes in both hemispheres to a depth of 9 mm (rats) or 10.5 mm (guinea pigs) from dura.

The femoral vein is exposed and a dose of a test compound is administered. About 7 min later a fluorescent dye (e.g., Evans Blue) is administered. The fluorescent dye complexes with proteins in the blood and functions as a marker for protein extravasation. Ten (10) min post-injection of the test compound, the left trigeminal ganglion is stimulated for 3 minutes at a current intensity of 1.0 mA (5 Hz, 4 msec duration) with a potentiostat/galvanostat. Fifteen minutes following stimulation, the animals are killed and exsanguinated with 20 mL of saline. The top of the skull is removed to facilitate the collection of the dural membranes. The membrane samples are removed from both hemispheres, rinsed with water, and spread flat on microscopic slides. Once dried, the tissues are coverslipped with a 70% glycerol/water solution. A fluorescence microscope equipped with a grating monchromator and a spectrophotometer is used to quantify the amount of fluorescent dye in each sample.

The extravasation induced by the electrical stimulation of the trigeminal ganglion is an ipsilateral effect (i.e. occurs only on the side of the dura in which the trigeminal ganglion is stimulated). This allows the other (unstimulated) half of the dura to be used as a control. The ratio of the amount of extravasation in the dura from the stimulated side, over the amount of extravasation in the unstimulated side, is calculated. Control animals dosed with only saline, yield, for example, a ratio of about 2.0 in rats and about 1.8 in guinea pigs. In contrast, a compound that effectively prevents the extravasation in the dura from the stimulated side yields a ratio of about 1.0. Dose-response curves can be generated for a test compound and the dose that inhibits the extravasation by 50% (ID₅₀) or 100% (ID₁₀₀) can be determined.

### Amygdala Kindling Model

A relationship has been reported between migraine, affective illness, and epilepsy. Although the three disorders are distinct, they all are paroxysmal dysregulations of the nervous system that partially overlap in their pharmacology. The kindling model for complex-partial seizures is based on the progressive development of seizures combined with electroencephalographic (EEG) paroxysmal patterns induced by repeated initially subconvulsive electrical stimulation of limbic structures, e.g., the basolateral nucleus of the amygdala. Once established, the phenomenon persists for months. Since the amygdala-kindled seizures in animals share numerous characteristics with complex-partial seizures in humans, it is a useful animal model of complex partial seizures (Loscher et al., Epilepsy Res. 1993, 15(3), 207-19). An advantage of using the amygdala kindling model is that both behavioral and EEG parameters of the partial and generalized seizures can be measured. Furthermore, the amygdala kindling model is reported to be appropriate for studying diseases such as migraine, affective illness and epilepsy which increase in severity overtime and in a manner which is related to the number of symptomatic episodes.

Rats are obtained at an age of 11-12 weeks (body weight 180-200 gm). Rats are maintained separately in plastic cages at controlled temperature (23° C.) and humidity (about 50% RH) with a 12-h light cycle. The rats receive standard diet and tap water ad libitum.

For implantation of stimulation and recording electrodes, rats are anesthetized and receive stereotaxic implantation of one bipolar electrode in the right basolateral amygdala. Coordinates for electrode implantation are AP-2.2 mm, L-4.8 mm, V-8.5 mm. All coordinates are measured from bregma. Skull screws serve as the reference electrode. The electrode assembly is attached to the skull by dental acrylic cement. After a postoperative period of 2 weeks, constant current stimulations (500 μA, 1 ms, monophasic square-wave pulses, 50/sec for 1 sec) are delivered to the amygdala at intervals of 1/day until ten stage 5 seizures are elicited. The electrical susceptibility of the stimulated region (threshold for induction of afterdischarges) is recorded on the first day of the experiment (initial afterdischarge threshold) as well as after kindling acquisition (with an interval of at least 4 days after the tenth stage 5 seizure) using an ascending staircase procedure. The initial current intensity is 1 μA, and the current intensity is increased in steps of about 20% of the previous current at intervals of 1 min until an afterdischarge of at least 3 sec duration is elicited. In addition to afterdischarge threshold, the following parameters of kindled seizures are measured in fully-kindled rats after stimulation with the afterdischarge threshold current: seizure severity is classified as follows: (1)immobility, eye closure, twitching of vibrissae, sniffing, facial clonus; (2) head nodding associated with more severe facial clonus; (3) clonus of one forelimb; (4) rearing, often accompanied by bilateral forelimb clonus; (5) rearing with loss of balance and falling accompanied by generalized clonic seizures. Seizure duration 1 is the duration of limbic (stage 1-2) and/or motor seizures (stage 3-5). Seizure duration 2 includes the time of limbic and/or motor seizures plus the adjacent time of immobility. Afterdischarge duration 1 (ADD 1) is the time of spikes in the EEG recorded from the site of stimulation with a frequency of at least 1/sec. Afterdischarge duration 2 (ADD 2) is the total time of spikes occurring in the EEG including those, which followed the ADD 1 with lower frequency and amplitude.

Test compound is administered to the prepared animals. Control experiments are performed 2-3 days before each test compound experiment. For control determinations, rats receive vehicle (e.g., saline) with the pretreatment time of the respective test compound experiment. For all test compound experiments, at least 4 days are interposed between 2 drug injections in order to avoid alterations in drug potency due to cumulation or tolerance. The data is analyzed using appropriate statistical methods.

In addition to recording anticonvulsant parameters, kindled rats can be observed for adverse effects in order to estimate a therapeutic index. Tests include open field observations, the rotarod test, and body temperature. Tests used to evaluate adverse effects are performed in the same manner in control and drug experiments at two different times, just before application of drug or vehicle and 13 min after application.

The rotarod test is carried out with a rod of 6 cm diameter and rotation speed of 8 rpm. Neurological deficit is indicated by inability of the animals to maintain their equilibrium for at least 1 min on the rotating rod. Rats are trained prior to the rotarod evaluation to maintain their balance on the rod. After treatment with a test compound or vehicle, rats that are not able to maintain their equilibrium on the rod for three subsequent 1 min attempts are considered to exhibit neurological deficit.

In addition to these quantitative estimations of neurological deficit, behavioral alterations after administration of test compound are noted in the cage and after placing the animals in an open field of 90-100 cm diameter. Muscle tone is estimated by palpation of the abdomen. The extent of deficits in behavior after administration of a test compound is determined by a rating system. Animals are removed from the cage, placed in an open field, observed for about 1 minute and rated separately for ataxia, abducted hindlimbs, reduced righting, flat body posture, circling, Straub tail, piloerection, hypolocomotion and hyperlocomotion (abdominal muscle tone is evaluated by palpation at the end of the period of observation). All other parameters except ataxia are scored from 0 to 3: (0) absent; (1) equivocal; (2) present; (3) intense. For ataxia: (1) slight ataxia in hind-legs (tottering of the hind quarters); (2) more pronounced ataxia with dragging of hind legs; (3) further increase of ataxia and more pronounced dragging of hind legs; (4) marked ataxia, animals lose balance during forward locomotion; (5) very marked ataxia with frequent loss of balance during forward locomotion; (6) permanent loss of righting reflexes, but animal still attempts to move forward. Rectal body temperature is measured. Body weight of the animals is recorded once daily before the test compound is administered. Data is analyzed by an appropriate statistical method. The ability of a test compound to the electrical threshold for induction of after discharges, decrease the severity of seizures, reduce seizure duration, and reduce total afterdischarge duration indicates efficacy in treating migraine.

### Clinical Trial Protocol

The efficacy of one or more compounds of Formula (I), Formula (II), Formula (III), or Formula (IV) in treating migraine may be assessed using a randomized, double-blind, placebo-controlled, parallel group, clinical trial. The primary objective of the study is to evaluate the safety and efficacy of a test compound versus placebo in the treatment of recurrent episodes of migraine based on change from the baseline phase to the double-blind phase in the monthly (28 days) migraine episode rate. The secondary objectives are to (a) evaluate the effect of treatment with a test compound versus placebo in migraine patients on percentage of subjects responding to treatment (50% or more reduction in monthly migraine episode rate) and change from the baseline phase to the double-blind phase in (b) migraine days per month, (c) average migraine duration, (d) rescue medication use, (e) average severity of migraine headache, (f) average severity of migraine associated symptoms (nausea, vomiting, photophobia, phonophobia); to provide safety and efficacy data for the comparison a dose of a test compound in the treatment of migraine; and to evaluate the effect of treatment with a dose of a test compound versus placebo in migraine patients on migraine-specific measures of health-related quality of life (HRQL) and SF-36 quality-of-life measures, as well as the correlation between HRQL and migraine frequency.

The clinical trial is a randomized, double-blind, placebo controlled, parallel-group, multicenter study to evaluate the efficacy and safety of one or more doses of a test compound versus placebo in migraine prophylaxis. Patients are randomized into treatment groups. The patients must have been diagnosed with migraine for at least twelve months, with or without aura, as defined by the International Headache Society (HIS). The IHS diagnostic criteria differ from the definition of a migraine period utilized in this study for evaluation of efficacy. For the purposes of this study a migraine period is defined as the twenty-four hour duration starting with the onset of painful migraine symptoms, or aura with successful abortive/rescue treatment. Any recurrence during the twenty-four hour period is considered part of the initial episode. If the migraine pain persists beyond the twenty-four hour period, for the purposes of this study, this is considered a new episode.

There are four phases in the clinical trial: Baseline, Core Double-Blind, Blinded Extension, and Taper/Exit.

The Baseline Phase lasts up to 42 days and included two periods: Washout and Prospective Baseline. At Baseline Visit 1 (screening), subjects are evaluated to ensure that they meet inclusion/exclusion criteria. In addition, a three-month retrospective headache history is recorded. During each of the three months prior to Visit 1, patients should have had no more than 8 migraines and no more than 15 total headache days (migraine plus other headache types). Eligible patients then undergo other study procedures and are given a headache/rescue medication record. Patients maintain this record from Visit 1 throughout their participation in the clinical trial, documenting the occurrence of any headaches, or auras, as well as the duration, severity and symptomatology of any migraine attacks. Patients also record the use of any abortive/rescue medication taken for the relief of migraine pain and associated symptoms, or during an aura to prevent migraine pain or relieve symptoms. In addition, for each migraine attack, patients answer the questions on the headache record regarding work loss and productivity.

If at the start of the trial, eligible patients are on any prophylactic medication to treat their migraines, they enter a Washout Period of up to 14 days to taper from these medications. This washout is from the time the patient enters the Prospective Baseline Period, 28 days prior to Visit 2 (randomization).

At Baseline Visit 2 (Day 1), headache/rescue medication record information is reviewed. To be eligible for randomization into the trial a patient must have had 3 to 12 migraine episodes but no greater than 15 (migraine and non-migraine), headache days during the 28 days prior to Visit 2.

In the Core Double-Blind Phase, patients who complete the Baseline Phase and meet the entry criteria (including Prospective Baseline Period migraine/headache rate) are randomized into treatment groups representing one or more doses of test compound or placebo. The Core Double-Blind Phase has two periods: Titration and Maintenance.

The Titration Period immediately follows the Baseline Phase and extends for eight weeks (56 days). During this period, patients randomized to test compound are started at an initial dose and the daily dose is increased weekly until the assigned dose is achieved (or maximum tolerated dose, whichever is less). From the third week of Titration until the end of the Maintenance Period, a maximum of two dose level reductions are permitted for unacceptable tolerability problems. If a patient is still in the Titration Period, after a dose reduction, rechallenge is attempted to approach the patient's assigned dose, and, if unsuccessful, the dose is reduced again to the original reduced dose. Patients who have already had their study medication dose decreased by two levels, and are still experiencing unacceptable tolerability problems, which warrant additional dose reductions, exit the study, or enter the Open Label Extension Phase, where their dose is further adjusted. Clinic visits occur on, for example, Day 29 (Visit 3) and Day 57 (Visit 4/End of Titration).

During the 18-week Maintenance Period, patients remain on the dose of test compound reached at the end of the Titration Period (the assigned dose or the maximum tolerated dose). If a patient experiences unacceptable tolerability problems, the dose is reduced, but only to the point that there are no more than two dose reductions for the entire Core Phase (Titration plus Maintenance). No rechallenge is permitted during the Maintenance Period, so a patient continues on the reduced dose for the remainder of the period. Patients who have already had their study medication dose decreased by two levels, and are still experiencing unacceptable tolerability problems, which would warrant additional dose reductions, exit the study. Clinic visits occur, for example, on Day 83 (Visit 5), Day 113 (Visit 6), Day 141 (Visit 7) and Day 183 (Visit 8/Core Double-Blind Final Visit or Early Withdrawal).

Patients are considered to have completed the Core Double-Blind Phase if they complete all 26 weeks of the Phase (8 weeks of Titration and 18 weeks of Maintenance) without prematurely discontinuing study medication. Only patients who complete all 26 weeks of the Core Phase have the option of entering the Blinded Extension Phase.

During the Blinded Extension Phase, patients remain on test compound at the same dose they achieved during the Core Phase for six months, or until they withdraw. During this phase, patients are not permitted to adjust the dose of test compound. Patients are seen quarterly during this phase (Visits 10 and 11/Blinded Extension Final Visit). Patients are considered to have completed the Blinded Extension Phase if they complete all six months of the Phase without prematurely discontinuing the test compound.

In the Taper/Exit Phase, patients exiting the study are tapered from study medication. If a patient exits the study during the Core Double-Blind Phase (Titration or Maintenance Period), he or she is tapered from study medication in a blinded fashion. The length of the taper is as long as seven weeks, but varied according to the dose the patient achieves. Patients who exit the study during the Blinded Extension Phase are tapered from their medication following the recommended taper schedule.

Physical examinations (including height) and neurologic examinations are performed at the beginning and end of the study. A baseline electrocardiogram is performed at the beginning of the study. Vital signs and weight are recorded at each clinic visit. Adverse events are recorded. Quality of Life assessments are performed at intervals, for example, Visits 2 (Day 1), 4 (Day 57/Exit from Titration), 6 (Day 113) and 8 (Day 183/Core Double-Blind Final Visit/Early Withdrawal). Health Care Resource Use information is recorded at intervals, for example, Visits 3 through 8. The occurrence of any headaches or auras, severity and symptomatology of any migraine headaches, and the use of rescue medication is transcribed from a patient's headache record to their case record form at each visit.

Efficacy evaluations are based on information recorded on the subject's headache/rescue medication record and Health-Related Quality of Life assessments. On the headache/rescue medication record the patients document the following throughout his/her study participation: occurrence and duration of headaches (and auras if no headache pain develops), severity of migraine pain and associated symptoms, as well as the use of medication taken to relieve migraine pain or symptoms (or taken during an aura to relieve symptoms or prevent migraine pain). Health-Related Quality of Life (HRQL) assessments are completed at specified intervals throughout the study. The Migraine-Specific Quality of Life questionnaire (MSQ), and the Medical Outcomes Study Short Form-36 (SF-36) can be used to assess HRQL.

The primary efficacy criterion is the reduction in migraine episodes per month (28 days) during the Core Double-Blind Phase compared to the 28 day Prospective Baseline Period. The secondary efficacy criteria include the percentage of patients responding to treatment (50% or more reduction in the monthly (28 day) migraine episode rate) and reduction from the Prospective Baseline Period to the Core Double-Blind Phase in (a) migraine days per month, (b) monthly rate of all types of headaches, (c) average migraine duration, (d) rescue medication use, (e) average severity of migraine headache, and (f) average severity of migraine-associated symptoms (nausea, vomiting, photophobia, phonophobia). Also included in the secondary efficacy criteria is the effect of treatment with test compound versus placebo on migraine-specific measures of health-related quality of life (HRQL) and SF-36 quality-of-life measures, as well as the correlation between HRQL and migraine frequency. The Medical Outcomes Study Short Form-36 (SF-36) is the most frequently used generic measure of HRQL in migraine patients and has been used in several studies of migraine. The SF-36 is a 36-item questionnaire measuring eight domains. The SF-36 has been shown to be reliable and valid in a wide variety of patient populations as well as for migraine patients. The migraine specific quality of life questionnaire (MSQ) can also be administered. The MSQ is a disease-specific instrument developed to assess quality of life relating to migraine. For example, the 2.1 version has 14 items within three domains. The MSQ has been used most often in published clinical trials of migraine therapy and has demonstrated evidence of reliability, validity, and responsiveness.

### Example 9

### Use of Animal Models to Assess the Efficacy of Compounds of Formulae (I)-(IV) for Treating Fibromyalgia

Efficacy for treating fibromyalgia may be evaluated using animal models of neuropathic pain known in the art such as, for example, the carrageenan-induced paw hyperalgesia model, the von Frey filament test, chronic constriction injury, the Chung model of rat neuropathic pain, the Hargreaves test, the cold allodynia model, as well as other tests.

The carrageenan-induced paw hyperalgesia test is a model of inflammatory pain. A subcutaneous injection of carrageenan is made into the left hind paws of rats. The rats are treated with a compound to be evaluated before the carrageenan injection (e.g., 30 minutes) or after the carrageenan injection (e.g., two hours). Paw pressure sensitivity for each animal is tested with an analgesymeter three hours after the carrageenan injection (see e.g., Randall et al., Arch. Int. Pharmacodyn. 1957, 111, 409-419).

The effects of test compounds on carrageenan-induced paw edema can also be examined. This test allows an assessment of the ability of a compound to reverse or prevent the formation of edema evoked by paw carrageenan injection (Vinegar et al., J. Phamacol. Exp. Ther. 1969, 166, 96-103). The paw edema test is carried out using a plethysmometer for paw measurements. After administration of a test compound, a carrageenan solution is injected subcutaneously into the lateral foot pad on the plantar surface of the left hind paw of an animal. At three hours post-carrageenan treatment, the volume of the treated hind paw (left) and the un-treated hind paw (right) is measured using a plethysmometer.

The effect of compounds on mechanical allodynia can be determined using the von Frey filament test in rats with a tight ligation of the L-5 spinal nerve. The von Frey test is recognized as a model of painful peripheral neuropathy. The surgical procedure is described by Kim et al., Pain 1992, 50, 355-363. The von Frey test is performed within the sciatic or saphenous innervation area of the hindpaws. A logarithmic series of 10 calibrated Semmes-Weinstein monofilaments is applied randomly to the right hind paws to determine the stimulus intensity threshold stiffness required to elicit a paw withdrawal response (Chaplan et al., J. Neurosci. Methods 1994, 53, 55-63). Flinching and licking of the paw and paw withdrawal on the ligated side are considered positive responses. Log stiffness of the hairs is determined by log₁₀ (milligrams×10). The range of monofilaments used in these experiments (0.407-15.136 gm) produce a logarithmically graded slope. Interpolated 50% response threshold data is expressed as stimulus intensity in log₁₀ (milligrams×10) or as gram fiber force. Assessments are made prior to (baseline) and at specific times after administration of test compound. Behavioral observers are blinded to treatment groups. Responses are used to calculate the 50% paw withdrawal threshold (absolute threshold) by fitting a Gaussian integral psychometric function using a maximum-likelihood fitting method and this fitting method allows parametric statistical analyses.

Heat and cold allodynia responses as well as mechanical allodynia sensations can be evaluated in rats having a chronic constriction injury (CCI). In the CCI model a unilateral mononeuropathy can be produced in anesthetized rats as described in Bennett et al., Pain 1988, 33, 87-107. The lateral aspect of each hind limb is shaved and scrubbed with Nolvasan. Using aseptic techniques, an incision is made on the lateral aspect of the hind limb at the mid-thigh level. The biceps femoris is bluntly dissected to expose the sciatic nerve. On the right hind limb of each rat, four loosely tied ligatures are made around the sciatic nerve approximately 1-2 mm apart. On the left side of each rat, an identical dissection is performed except that the sciatic nerve is not ligated (sham). The muscle and the overlying skin is closed. The responses of the CCI rats can then be determined using a model of heat or cold allodynia.

In the Chung model of rat neuropathic pain heat and cold allodynia responses as well as mechanical allodynia sensations are evaluated as described infra in rats following spinal nerve injury (e.g. ligation, transaction). The Chung model is initially described in Kim and Chung, Pain 1992, 50, 355-363.

The Hargreaves test is another radiant heat model for pain (Hargreaves et al., Pain 1998, 32, 77-88). CCI rats are tested for thermal hyperalgesia at least 10 days post-operation. The test apparatus comprises an elevated heated (80-82° F.) glass platform. Eight rats at a time, representing all testing groups, are confined individually in inverted plastic cages on the glass floor of the platform at least 15 minutes before testing. A radiant heat source placed underneath the glass is aimed at the plantar hind paw of each rat. The application of heat is continued until the paw is withdrawn (withdrawal latency) or the time elapsed is 20 seconds. This trial is also applied to the sham operated leg. Two to four trials are conducted on each paw, alternately, with at least 5 minutes interval between trials. The average of these values represents the withdrawal latency.

The test apparatus and methods of behavioral testing for the cold allodynia model are described in Gogas et al., Analgesia 1997, 3, 111-118. The apparatus for testing cold allodynia in neuropathic (CCI) rats comprises of a Plexiglass chamber with a metal plate 6 cm from the bottom of the chamber. The chamber is filled with ice and water to a depth of 2.5 cm above the metal plate, with the temperature of the bath maintained at 0-4° C. throughout the test. Each rat is placed into the chamber individually, a timer started, and the animal's response latency is measured. A “response” is defined as a rapid withdrawal of the right ligated hind paw completely out of the water when the animal is stationary and not pivoting. An exaggerated limp while the animal is walking and turning is not scored as a response. Baseline scores for withdrawal of the ligated leg from the water typically range from 7-13 seconds. The maximum immersion time is 20 seconds with a 20-minute interval between trials.

These and other models of neuropathic pain are disclosed, for example, in Bennett and Xie, Pain 1988, 33, 87-107; Chaplan et al., J. Neurosci. Meth. 1994, 53, 55-63; Fox et al., Pain 2003, 105, 355-362; Milligan et al., Brain Res. 2000, 861, 105-116; De Vry et al., Eur. J. Pharmacol. 2004, 491, 137-148; and Polomano et al., Pain 2001, 94, 293-304.

### Example 10

### Use of Animal Models to Assess the Efficacy of Compounds of Formulae (I)-(IV) for Treating Irritable Bowel Syndrome

Increase of defecation number induced by restraint stress can be used as a pathological model of irritable bowel syndrome (Miyata et al., J. Pharmacol. Exp. Ther 1992, 261, 297-303). Using the method of Williams et al., the effectiveness of test compounds on reducing the defecation number in rats is evaluated (Williams et al., Gastroenterology 1988, 94, 611-621). A test compound is administered to rats, and after 1 hour, their forelegs are fixed on their trunks with adhesive tape under ether anesthesia (load of restraint stress). One hour after affixing the restraint, defecation number is counted and the number compared with that of a control group. Due to the load of the restraint stress, the defecation number of rats in the control group increases significantly. Compounds showing usefulness in treating IBS reduce the increase in defecation number induced by the restraint stress.

A zymosan-induced hyperalgesia model has also been shown useful for evaluating the efficacy of treatment for IBS. Intracolonic instillation of zymosan, a yeast cell wall derivative, which acts as an inflamogen, produces colonic inflammation and enhanced visceromotor responses to colorectal distention as a measurement of response to pain (Coutinho et al., Brain Res 1996, 736, 7-15).

To surgically prepare rats in the hyperalgesia model, rats are first deeply anesthetized. Electrodes are stitched into the external oblique musculature for electromyographic (EMG) recording. Electrode leads are tunneled subcutaneously and exteriorized at the nape of the neck for future access. After surgery, the rats are housed separately and allowed to recuperate for at least 3 days prior to testing.

At the time of the test, the descending colon and rectum are distended by pressure-controlled inflation of a 7-8 cm-long flexible latex balloon tied around a flexible tube. The balloon is lubricated, inserted into the colon via the anus, and anchored by taping the balloon catheter to the base of the tail. Noxious phasic colorectal distension (CRD, 80 mmHg, 20 sec) is achieved by opening a solenoid gate to a constant pressure air reservoir. Intracolonic pressure is continuously monitored by the aid of a pressure control device. The response, quantified as the visceromotor response (VMR), is a contraction of the abdominal and hind limb musculature. EMG activity produced by contraction of the external oblique musculature is quantified. Each distension trial lasts for about 60 sec, and EMG activity is quantitated before, during, and after distension. The increase in EMG activity during distention is defined as the response.

Stable baseline responses to CRD (80 mmHg, 20 sec, 4 min apart) are obtained in conscious, unsedated rats before treatment, followed by oral gavage with a test compound. Control animals receive vehicle only. Sixteen (16) h following administration, a pre-zymosan response to distention is measured, followed by additional doses of test compound. The animals are then briefly anesthetized with halothane, and zymosan (1 mL, 25 mg/mL) is instilled into the colon with a gavage needle inserted to a depth of about 7-8 cm, to produce inflammation and enhance the VMR to CRD. Four hours after intracolonic treatment, responses to CRD are quantified as described in the preceding paragraph.

The efficacy of a compound for treating IBS may also be assessed using other distention models of visceral pain. A variety of assays can be used to assess visceromotor and pain responses to rectal distension (see e.g., Gunter et al., Physiol. Behav. 2000, 69(3), 379-82; Depoortere et al., J. Pharmacol. and Exp. Ther. 2000, 294(3), 983-990; Morteau et al., Fund. Clin. Pharmacol. 1994, 8(6), 553-62; Gibson et al., Gastroenterology 2001, (Suppl. 1), 120(5), A19-A20; and Gschossmann et al., Eur. J. Gastro. Hepat. 2002, 14(10), 1067-72. Visceral pain can lead to visceral reactions, which can manifest as, for example, contractions of the abdominal muscles. The number of contractions of the abdominal muscles occurring after a mechanical pain stimulus produced by distending the large intestine can thus be used as a measurement for determining visceral sensitivity to pain. For example, the ability of a test compound to reverse acetic acid-induced colonic hypersensitivity in a rodent model can be used to assess the efficacy of a test compound for treating irritable bowel syndrome. Adult rats are housed in an animal facility at standard conditions. Following one week of acclimatization to the animal facility, the rats are brought to the laboratory and handled daily for another week for the purposes of accommodation. The visceromotor behavioral response to colorectal distension is measured by counting the number of abdominal contractions recorded by a strain gauge sutured onto the abdominal musculature in awake, unrestrained animals as described in Gunter et al., Physiol. Behav. 2000, 69(3). 379-82. A 5 cm latex balloon catheter inserted via the anal canal into the colon is used for colorectal distensions. Constant pressure tonic distensions are performed in a graded manner (15,_{—}30, or 60 mmHg) and are maintained for a period of 10 min and the numbers of abdominal muscle contractions are recorded to measure the level of colonic sensation. A 10 min recovery is allowed between distensions.

Acetic acid-induced colonic hypersensitivity in rats is described by Langlois et al., Eur. J. Pharmacol. 1996, 318, 141-144; and Plourde et al., Am. J. Physiol. 1997, 273,G191-G196. For example, a low concentration of acetic acid (1.5 ml, 0.6%) is administered intracolonically to sensitize the colon without causing histological damage to the colonic mucosa. Test compound or vehicle alone is administered to the rats 30 min prior to initiation of the protocol for colorectal distension. Three consecutive colorectal distensions at 15, 30, or 60 mmHg applied at 10-min intervals are recorded. Visceromotor responses are evaluated as the number of abdominal muscle contractions recorded during the 10-min periods of colorectal distension. Non-sensitized and sensitized uninjected control animals serve to demonstrate the lower and upper levels of response, respectively. The ability of a test compound to significantly reduce colorectal sensitization-induced increases in visceromotor responses to colorectal distention in the animals tested can be predictive of the effectiveness of the test compound in treating IBS in humans.

The ability of a compound to suppress ovalbumin-induced bladder contractions in sensitized animals has also been shown to be a useful model for inflammatory gastrointestinal conditions such as IBS (see e.g., Ahluwalia et al., Br. J. Pharmacol. 1998, 124, 190-6). Sprague-Dawley rats are utilized for this study. The animals are divided into groups. A first group serves as the control and a second group is sensitized with ovalbumin (OA). Other groups are also sensitized as described but subjected to oral gavaging test compound prior to acute ovalbumin challenge. Sensitization of the animals is accomplished with an intraperitoneal injection of a mixture of 1 mg OA and 100 mg aluminum hydroxide suspended in 1 mL of saline. Fourteen days later, these sensitized rats are anaesthetized with a subcutaneous injection of urethane (1.2 g/kg) for intravesical OA (10 mg/mL) administration and evaluation of bladder hyperactivity. The animals in the first group receive saline (control), the second group of animals receive about 2 mL of OA (10 mg/mL OA in sterile saline), and other animal groups receive oral test compound 60 min prior to acute OA challenge (2 mL of 10 mg/mL OA).

To evaluate bladder overactivity in the animals, a 1 cm incision is made along the centerline of the lower ventral abdomen. The bladder is exteriorized, and catheterized by means of a polyethylene tube inserted into the bladder dome and sutured in place using a 2-0 braided silk suture. The bladder is returned to the abdomen, with the catheter line escaping through the incision. The catheter is then connected to a pressure transducer and connected to an infusion pump. During the continuous filling bladder cystometry, the pressure is recorded with the transducer.

To obtain the cystometry measurements, the bladder is first infused with warm 0.9% saline (37° C.) at 40 μL/min (2.4 mL/hr) and at least 20 minutes of stable voiding cycles are recorded during infusion measurements (Chuang et al., Urology 2003, 61, 664-70). This procedure is followed by intravesical infusion of OA (10 mg/mL) and bladder contractions are recorded. Frequency of contractions (voids), inter-contractile interval (ICI), and non-voiding contractions (NVC) are calculated from these recordings. Inhibition of bladder smooth muscle contractions, which lead to an increase in NVC and a decrease in ICI induced by acute intravesical challenge of OA-sensitized animals can indicate usefulness in treating IBS.

### Example 11

### Use of Animal Models to Assess the Efficacy of Compounds of Formula (I), Formula (II), Formula (III), and Formula (IV) for Treating Social Phobia

Animal models may be used for assessing the efficacy of a compound in treating an affective disorder such as social phobia.

### Fear-Potentiated Startle Model

The fear-potentiated startle paradigm, e.g., increased startle in the presence of a conditioned fear stimulus (CFS), is a learned fear paradigm that has been shown to involve the central amygdala (see, e.g., Davis, Behav. Neurosci. 1986, 100, 814-824; and Helton et al., J. Pharmacol. Exp. Ther 1998, 284, 651-660). Fear potentiated startle evokes a neurological process that mimics the behavioral pathology manifest in post-traumatic stress disorder and other anxiety-based diseases. Elevated anxiety impairs sensory processing with consequent deterioration of memory, cognition, and social function. Anxiogenic states seen in human conditions such as generalized social phobia (Stein et al., Arch. Gen. Psychiatry 2002, 59, 1027-1034) or drug-induced animal models of anxiety (Sanders and Shekhar, Pharmacol. Biochem. Behav. 1995, 52, 701-706) are accompanied by abnormal amygdala function. Human studies have demonstrated that both the baseline and fear-potentiated responses can be inhibited by anxiolytic drugs such as the benzodiazepine, alprazolam (Riba et al., Psychopharmacology (Berl) 2001, 157, 358-367). Measures of fear-potentiated startle response in rats and humans provide an indication for the potential anxiolytic activity of a drug (see e.g., Belzung, Current Opinion in Investigational Drugs. 2001, 2(8), 1108-1111; and Nestler et al., Neuron, 2002, 34, 13-25). Thus, these known models can be used to confirm the efficacy of one or more compounds of Formula (I), Formula (II), Formula (III), and Formula (IV) as therapies for affective disorders.

Male Sprague-Dawley rats weighing 350-450 g are used. Animals are maintained on a 12:12 hour light-dark cycle with food and water continuously available. Animals are trained and tested in 8×15×15 cm³ Plexiglas™ and wire-mesh cages. Each cage floor consists of four 6 mm diameter stainless-steel bars spaced 18 mm apart. Each cage is suspended between compression springs within a steel frame and located within a 90×70×70 cm³ ventilated sound-attenuating chamber. Background noise (60 dB wide-band) is provided by a noise generator and delivered through high-frequency speakers located 5 cm in front of each cage. Sound level measurements (sound pressure level) are made with a sound-level meter (A scale; random input) with a microphone located 7 cm from the center of the speaker (approximating the distance of the rat's ear from the speaker).

Startle responses are evoked by 50 msec, 95 dB white noise bursts (5 msec rise-decay) delivered through the same speakers used to provide background noise. An accelerometer affixed to the bottom of each cage produces a voltage output proportional to the velocity of cage movement. This output is amplified and digitized. Startle amplitude is defined as the maximal peak-to-peak voltage that occurs during the first 200 msec after onset of the startle-eliciting stimulus.

The conditioned stimulus (CS) is a 3.7 sec light (80 lux) produced by an 8 W fluorescent bulb (100 msec rise time) located 10 cm behind each cage. Luminosity is measured using a light meter. A second stimulus is a 0.5 second shock, delivered to the floorbars and produced by a shock generator. Shock intensities (measured as in, for example, Cassella et al., Physiol Behav 1986, 36, 1187-91) are 0.4 mA.

Test compounds are administered prior to evaluation. To match the animals for assessment, on each of the next 2 days animals are placed in the test chambers and presented with 30 95 dB noise bursts at a 30 sec interstimulus interval (ISI). The mean startle amplitude across the 30 stimuli on the second day is used to divide rats into groups with similar startle amplitudes.

To fear condition the test animals, on each of the next 2 days, rats are returned to the test chambers and 5 min later given the first of 10 light-footshock pairings. The 0.4 mA 0.5 sec shock is delivered during the last 0.5 sec of the 3.7 sec light. The average intertrial interval (ITT) is 4 min (range, 3-5 min).

Twenty-four (24) hours after the last fear conditioning session, test compound or vehicle is administered to the rats and the rats are immediately placed into the test chambers. After 5 min the rats receive 30 95 dB noise bursts (30 sec ISI) to habituate the startle response to a stable baseline prior to the test trials. Each test trial (18 total) involves the presentation of a noise burst of one of 3 intensities (95, 100, or 105 dB); half of these occurring in the presence and half in the absence of the light CS. On the CS trials the startle stimulus is presented 3.2 sec after the onset of the 3.7 sec light. Trial types are presented in a balanced, irregular order (30 sec ITI) with the restriction that each of the 6 trial types occur once within each of the 3 trial blocks.

The initial startle stimuli of the test session are used to habituate startle responses to asymptotic levels and are not included in statistical analyses. Subsequent startle responses generated by the three different startle intensities are averaged for each animal to obtain a single score for both the startle stimulus alone (baseline) and the CS and startle stimulus trials. A difference score is computed for each animal by subtracting the mean baseline startle amplitudes from the mean startle amplitudes in the presence of the CS. Analysis of the data is performed using appropriate statistical methods.

### Activity and Functional Observation Measurements

Rats are dosed with vehicle or test compound from postnatal day 25 to post natal day 70. The locomotor activity of 10 randomly selected rats/sex/group is measured on postnatal day 30 (adolescent) and postnatal day 72 (adult). On postnatal day 30 and postnatal day 72, each rat is placed in a shoebox cage equipped with the automated Photobeam Activity System. Locomotor activity is monitored during a 60 min session composed of 12, 5-min intervals. The total number of photobeam breaks that occur during each of the 12, 5-min intervals is recorded. Changes in habituation to novel environments are assessed by comparing locomotor activity over 3 session intervals between the control versus test groups for habituation. Emotionality is determined by following behavioral facets including defecation, urination, rearing, grooming, and backing (see e.g., Hall, J. Comp. Physiol. Psychol., 1936, 22, 325-352; and Spyker, in Behavioral Toxicology, Ed. Weiss and Laties, Plenum Press, New York, pp 311-349, 1975). A functional observation assessment is performed on postnatal day 75 according to the parameters described by Irwin, Psychopharmacologia 1968, 13, 222-257, to evaluate gait, posture, abnormal behavior, and vocalization.

### Spatial Navigation in an M Swim Maze

Drugs with anxiolytic or anti-depression activity often demonstrate unwanted side-effects, such as sedation, amnesia or other cognitive impairment, hyperactivity, or hypoactivity. A standard test for these unwanted side-effects is to quantify activity and emotionality to a novel surrounding in rats after repeated exposure to drug. An additional test to measure effects of a drug on aspects of learning and memory is the M Swim Maze.

The M Swim Maze was developed to test spatial learning and memory (e.g., functional memory). The animal has no visual or spatial cues in the pool and must rely on extra-maze cues (e.g., light setup outside the pool that can be seen by the swimming animal). Through a series of trials a rat develops “place learning” or knowledge about the position of the escape platform based upon the extra-maze cues. The platform can be moved to a different arm of the M configuration each day, combining spatial memory with working memory. This paradigm involves extinction of the prior memory and resolution of a new spatial problem. Many drugs that have anxiolytic or anti-depressive effects have detrimental effects on functional memory important for daily life. Additionally, spatial learning and memory tasks in rodents during stressful activities, such as escape from water, are useful to evaluate drugs for unwanted side effects of impairment of functional memory. Results in rodents correlate well with those in humans and other mammals. Decreased performance in this model indicates a negative locomotor or cognitive side-effect of drug treatment. An improvement may indicate improved cognition due to reduced stress or anxiety from task performance.

As an example, vehicle or test compound is administered to rats on postnatal day 25 through postnatal day 70 (45 days). Learning and memory are evaluated in a water M-maze. The evaluation consists of 10 trials/day for each animal on 4 successive days to assess short-term memory. The animals are evaluated for their ability to escape from the maze via a platform located on the lighted arm of an M-shaped maze. After placement of the animal in the central arm of the M-shaped maze, the goal side is varied for each animal at each trial according to a predetermined computer generated sequence. The same animals are also tested 5 days after the initial testing to assess long-term memory. On each day each animal is allowed 10 trials in the maze and time to escape is measured. Analysis of the data is performed using appropriate statistical analysis methods.

### Social Interaction Test

The Social Interaction Test is another test that can be used to assess anxiolytic properties (see e.g., File and Hyde, Pharmacol Biochem Behav 1979, July 11(1), 65-69).

Rats are allowed to acclimate to the animal care facility for 5 days and are housed singly for 5 days prior to testing with free access to food and water. Animals are handled for 5 min per day. The Social Interaction Test can be performed as described by Kennett, et al. Neuropharmacology 1997, 36(4-S), 601-608). On the test day, weight matched pairs of rats, unfamiliar to each other, are given identical treatments and returned to their home cages. Animals are randomly divided into treatment groups and are administered test compound, vehicle, or chlordiazepoxide (5 mg/kg). Dosing is at least 1 h prior to testing. Rats are subsequently placed in a white Perspex test box or arena (54×37×26 cm³) in which the floor is divided up into 24 equal squares, for 15 min. Background noise is applied. Sessions are videotaped. Active social interaction, defined as time involved in grooming, sniffing, biting, boxing, wrestling, following, and crawling over or under, is scored. The number, of episodes of rearing (animal completely raises its body on its hind limbs), grooming (licking, biting, scratching of body), and face washing (i.e., hands are moved repeatedly over face), and number of squares crossed are scored. Passive social interaction (animals lying beside or on top of each other) is not scored. The social interaction data is analyzed by appropriate statistical methods.

### Example 12

### Use of Animal Models to Assess the Efficacy of Compounds of Formula (I), Formula (II), Formula (III), and Formula (IV) for Treating Amyotrophic Lateral Sclerosis

A murine model of SOD 1 mutation-associated ALS has been developed in which mice express the human superoxide dismutase (SOD) mutation glycine.fwdarw.alanine at residue 93 (SOD1). These SOD1 mice exhibit a dominant gain of the adverse property of SOD, and develop motor neuron degeneration and dysfunction similar to that of human ALS (Gurney et al., Science 1994, 264(5166), 1772-1775; Gurney et al., Ann. Neurol. 1996, 39, 147-157; Gurney, J. Neurol. Sci. 1997, 152, S67-73; Ripps et al., Proc Natl Acad Sci U.S.A. 1995, 92(3), 689-693; and Bruijn et al., Proc Natl Acad Sci U.S.A. 1997, 94(14), 7606-7611). The SOD1 transgenic mice show signs of posterior limb weakness at about 3 months of age and die at 4 months. Features common to human ALS include astrocytosis, microgliosis, oxidative stress, increased levels of cyclooxygenase/prostaglandin, and, as the disease progresses, profound motor neuron loss.

Studies are performed on transgenic mice overexpressing human Cu/Zn-SOD G93A mutations (B6SJL-TgN (SOD1-G93A) 1 Gur) and non-transgenic B6/SJL mice and their wild litter mates. Mice are housed on a 12-h day/light cycle and (beginning at 45 d of age) allowed ad libitum access to either test compound-supplemented chow, or, as a control, regular formula cold press chow processed into identical pellets. Genotyping can be conducted at 21 days of age as described in Gurney et al., Science 1994, 264(5166), 1772-1775. The SOD1 mice are separated into groups and treated with a test compound or serve as controls.

The mice are observed daily and weighed weekly. To assess health status mice are weighed weekly and examined for changes in lacrimation/salivation, palpebral closure, ear twitch and pupillary responses, whisker orienting, postural and righting reflexes and overall body condition score. A general pathological examination is conducted at the time of sacrifice.

Motor coordination performance of the animals can be assessed by one or more methods known to those skilled in the art. For example, motor coordination can be assessed using a neurological scoring method. In neurological scoring, the neurological score of each limb is monitored and recorded according to a defined 4-point scale: (0) normal reflex on the hind limbs (animal will splay its hind limbs when lifted by its tail); (1) abnormal reflex of hind limbs (lack of splaying of hind limbs weight animal is lifted by the tail); (2) abnormal reflex of limbs and evidence of paralysis; (3) lack of reflex and complete paralysis; and (4) inability to right themselves when placed on the side sin 30 seconds or found dead. The primary end point is survival with secondary end points of neurological score and body weight. Neurological score observations and body weight are made and recorded five days per week. Data analysis is performed using appropriate statistical methods.

The rotarod test evaluates the ability of an animal to stay on a rotating dowel allowing evaluation of motor coordination and proprioceptive sensitivity. The apparatus is a 3 cm diameter automated rod turning at, for example, 12 rounds per min. The rotarod test measures how long the mouse can maintain itself on the axle without falling. The test can be stopped after an arbitrary limit of 120 sec. Should the animal fall down before 120 sec, the performance is recorded and two additional trials are performed. The mean time of 3 trials is calculated. A motor deficit is indicated by a decrease of walking time.

In the grid test, mice are placed on a grid (length 37 cm, width 10.5 cm, mesh size 1×1 cm²) situated above a plane support. The number of times the mice put their paws through the grid is counted and serves as a measure for motor coordination.

The hanging test evaluates the ability of an animal to hang on a wire. The apparatus is a wire stretched horizontally 40 cm above a table. The animal is attached to the wire by its forepaws. The time needed by the animal to catch the string with its hind paws is recorded (60 sec max) during three consecutive trials.

Electrophysiological measurements (EMG) can also be used to assess motor activity condition. Electromyographic recordings are performed using an electromyography apparatus. During EMG monitoring the mice are anesthetized. The measured parameters are the amplitude and the latency of the compound muscle action potential (CMAP). CMAP is measured in gastrocnemius muscle after stimulation of the sciatic nerve. A reference electrode is inserted near the Achilles tendon and an active needle placed at the base of the tail. A ground needle is inserted on the lower back of the mice. The sciatic nerve is stimulated with a single 0.2 msec pulse at supramaximal intensity (12.9 mA). The amplitude (mV) and the latency of the response (ms) are measured. The amplitude is indicative of the number of active motor units, while distal latency reflects motor nerve conduction velocity.

The efficacy of test compounds can also be evaluated using biomarker analysis. To assess the regulation of protein biomarkers in SOD1 mice during the onset of motor impairment, samples of lumbar spinal cord (protein extracts) are applied to ProteinChip Arrays with varying surface chemical/biochemical properties and analyzed, for example, by surface enhanced laser desorption ionization time of flight mass spectrometry. Then, using integrated protein mass profile analysis methods, data is used to compare protein expression profiles of the various treatment groups. Analysis can be performed using appropriate statistical methods.

### Example 13

### Use of Clinical Trials to Assess the Efficacy of Compounds of Formula (I), Formula (II), Formula (III), and Formula (IV) for Treating Parkinson's Disease

The following clinical study may be used to assess the efficacy of a compound in treating Parkinson's disease.

Patients with idiopathic PD fulfilling the Queen Square Brain Bank criteria (Gibb et al., J Neurol Neurosurg Psychiatry 1988, 51, 745-752) with motor fluctuations and a defined short duration GABA analog response (1.5-4 hours) are eligible for inclusion. Clinically relevant peak dose dyskinesias following each morning dose of their current medication are a further pre-requisite. Patients are also required to have been stable on a fixed dose of treatment for a period of at least one month prior to starting the study. Patients are excluded if their current drug regime includes slow-release formulations of L-Dopa, COMT inhibitors, selegiline, anticholinergic drugs, or other drugs that could potentially interfere with gastric absorption (e.g. antacids). Other exclusion criteria include patients with psychotic symptoms or those on antipsychotic treatment patients with clinically relevant cognitive impairment, defined as MMS (Mini Mental State) score of less than 24 (Folstein et al., J Psychiatr Res 1975, 12, 189-198), risk of pregnancy, Hoehn & Yahr stage 5 in off-status, severe, unstable diabetes mellitus, and medical conditions such as unstable cardiovascular disease or moderate to severe renal or hepatic impairment. Full blood count, liver, and renal function blood tests are taken at baseline and after completion of the study.

A randomized, double-blind, and cross-over study design is used. Each patient is randomized to the order in which either LD/DC or one of the two dosages of test compound is administered in a single-dose challenge in double-blind fashion in three consecutive sessions. Patients are admitted to a hospital for an overnight stay prior to administration of test compound the next morning on three separate occasions at weekly intervals. After withdrawal of all antiparkinsonian medication from midnight the previous day, test compound is administered at exactly the same time in the morning in each patient under fasting conditions.

Patients are randomized with respect to the order of the days on which they receive placebo or test compound. The pharmacokinetics of a test compound can be assessed by monitoring plasma GABA analog concentration over time. Prior to administration, a 22 G intravenous catheter is inserted in a patient's forearm. Blood samples of 5 mL each are taken at baseline and 15, 30, 45, 60, 75, 90, 105, 120, 140, 160, 180, 210, and 240 minutes after administering a test compound or until a full off-state has been reached if this occurs earlier than 240 minutes after administration. Samples are centrifuged immediately at the end of each assessment and stored deep frozen until assayed. Plasma GABA analog levels are assessed by high-pressure liquid chromatography (HPLC). On the last assessment additional blood may be drawn for routine hematology, blood sugar, liver, and renal function.

For clinical assessment, motor function is assessed using UPDRS (United Parkinson's Disease Rating Scale) motor score and BrainTest (Giovanni et al., J Neurol Neurosurg Psychiatry 1999, 67, 624-629), which is a tapping test performed with the patient's more affected hand on the keyboard of a laptop computer. These tests are carried out at baseline and immediately following each blood sample until patients reach their full on-stage, and thereafter at 3 intervals of 20 min, and 30 min intervals until patients reach their baseline off-status. Once patients reach their full on-state, video recordings are performed three times at 20 min intervals. The following mental and motor tasks, which have been shown to increase dyskinesia (Duriff et al., Mov Disord 1999, 14, 242-245) are monitored during each video session: (1) sitting still for 1 minute; (2) performing mental calculations; (3) putting on and buttoning a coat; (4) picking up and drinking from a cup of water; and (5) walking. Videotapes are scored using, for example, versions of the Goetz Rating Scale and the Abnormal Involuntary Movements Scale to document a possible increase in test compound induced dyskinesia.

Actual occurrence and severity of dyskinesia is measured with a Dyskinesia Monitor (Manson et al., J Neurol Neurosurg Psychiatry 2000, 68, 196-201). The device is taped to a patient's shoulder on their more affected side. The monitor records during the entire time of a challenging session and provides a measure of the frequency and severity of occurring dyskinesias.

Results can be analyzed using appropriate statistical methods.

### Example 14

### Use of a Clinical Trial o Assess the Efficacy of Compounds of Formula (I), Formula (II), Formula (III), and Formula (IV) for Treating Asthma

Adult subjects (nonsmokers) with stable mild-to-moderate asthma are enrolled (see, e.g., Van Schoor and Pauwels, Eur Respir J 2002, 19, 997-1002). A randomized, double-blind, placebo-controlled, two-period crossover design is used. On screening day 1, patients undergo a methacholine challenge (<8 mg/mL). The baseline forced expiratory volume in one second (FEV1) prior to each subsequent challenge must be within 15% of the screening baseline FEV1 obtained at the first visit. A neurokinin challenge (1×10-6 mol/mL) on screening day 2 is performed 24-72 h later. Study-period one commences within 10 days after visit two. First, a methacholine and a neurokinin-A (NKA) challenge is performed on days 1 and 0, respectively. At visit four, test compound is administered at an appropriate dose and for an appropriate period of time. On the last 2 days of the treatment period, methacholine and NKA challenges are repeated. Following treatment-period one, there is a washout period of about 5 weeks, following which the patients crossed over to another medication or placebo in study period two, which is identical to period one. Pulmonary function tests are performed using a spirometeor. The metacholine challenge is performed by inhaling doubling concentrations of methacholine until the FEV1 falls by >20% of the postdiluent baseline FEV1 of that day as described by Cockcroft et al., Clin Allergy 1977, 7, 235-243. NKA challenge is performed by inhaling increasing concentrations of NKA as described by Van Schoor et al., Eur Respir J 1998, 12, 17-23. The effect of a treatment on airway responsiveness is determined using appropriate statistical methods.

### Example 15

### Use of Animal Models and Clinical Trials to Assess the Efficacy of Compounds of Formula (I), Formula (II), Formula (III), and Formula (IV) for Treating Cough

Male guinea pigs are individually placed into a sealed perspex exposure chamber and allowed to acclimatize prior to adminstration of tussive stimuli or test compound by aerosol. Cough responses are induced by exposure to an aerosol of either citric acid (20%, 10 min) or capsaicin (15 μM, 4 min) at flow rates of 2 L/min and 3 L/min, respectively. An observer continuously monitors the animals, and the number of coughs counted over a 15 min period from commencement of the aerosol administration of the tussive stimuli. Guinea pigs are then randomly allocated to receive either test compound or control, and exposure to the tussive stimuli repeated and the number of coughs recorded.

### Human Model

Healthy, nonsmoking subjects who do not experience symptoms of respiratory tract infection or seasonal allergy for at least 4 weeks prior to evaluation and who demonstrate normal pulmonary function are enrolled. Subjects inhale single breaths of capsaicin solution (ranging from 0.98 μmol/L to 1,000 μmol/L) from a compressed-air driven nebulizer controlled by a dosimeter. Single breaths of capsaicin solution are given in ascending order, with inhalations of saline solution randomly interspersed to increase challenge blindness, until the concentration inducing five or more coughs is reached. Breaths are delivered at 1-min intervals. The number of coughs in response to each concentration of capsaicin during the 1-min period immediately after each inhalation is recorded by a blinded observer. Subjects are unaware that the end point of the study is the number of coughs induced. After undergoing baseline capsaicin cough challenge, subjects are randomly assigned, in a double-blind manner, and administered a test compound at an appropriate dose or placebo, after which the cough challenge is repeated. A significant response can be defined as a/fourfold or greater increment in the capsaicin concentration required to elicit five or more coughs.

### Example 16

### Use of an Animal Model to Assess the Efficacy of Compounds of Formula (I), Formula (II), Formula (III), and Formula (IV) for Treating Chronic Obstructive Pulmonary Disease

An animal model using mice chronically exposed to cigarette smoke can be used for assessing efficacy in treating emphysema in mice chronically exposed to cigarette smoke is used (see, e.g., Martorana et al, Am J Respir Crit Care Med, 2005, 172, 848-835; and Cavarra et al., Am J Respir Crit Care Med 2001, 164, 886-890). Six-week old C57B1/6J male mice are used. In the acute study, the mice are exposed either to room air or to the smoke of five cigarettes for 20 minutes. In the chronic study, the mice are exposed to either room air or to the smoke three cigarettes/day for 5 days/week for 7 months.

For the acute study, mice are divided into three groups of 40 animals each. These groups are then divided into four subgroups of 10 mice each as follows: (1) no treatment/air-exposed; (2) no treatment/smoke-exposed; (3) an first dose of test compound plus smoke-exposed; and (4) a second dose of test compound. In the first group, trolox equivalent antioxidant capacity is assessed at the end of the exposure in bronchoalveolar lavage fluid. In the second group, cytokines and chemokines are determined in bronchoalveolar lavage fluid using a commercial cytokine panel at 4 hours; and in the third group bronchoalveolar lavage fluid cell count is assessed at 24 hours.

For the chronic study, five groups of animals are used: (1) no treatment/air-exposed; (2) a first dose of a test compound plus air-exposed; (3) no treatment/smoke-exposed; (4) a second dose of the test compound plus smoke-exposed; and (5) the first dose of the test compound plus smoke exposed. Seven months after chronic exposure to room air or cigarette smoke, 5 to 12 animals form each group are killed an the lungs fixed intratracheally with formalin. Lung volume is measured by water displacement. Lungs are stained. Assessment of emphysema includes mean linear intercept and internal surface area. The volume density of macrophages, marked immunohistochemically with antimouse Mac-3 monoclonal antibodies is determined by point counting. A mouse is considered to have goblet cell metaplasia when at least one or more midsize bronchi/lung showed a positive periodic acid-Schiff staining. For the determination of desmosine, fresh lungs are homogenized, processed, and analyzed by high-pressure liquid chromatography.

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

1. A method of treating a disease chosen from fibromyalgia, amyotrophic lateral sclerosis, social phobia, Parkinson's disease, asthma, cough, or chronic obstructive pulmonary disease in a patient, comprising administering to a patient in need of such treatment a therapeutically effective amount of at least one compound chosen from Formula (I), Formula (II), Formula (III), Formula (IV): 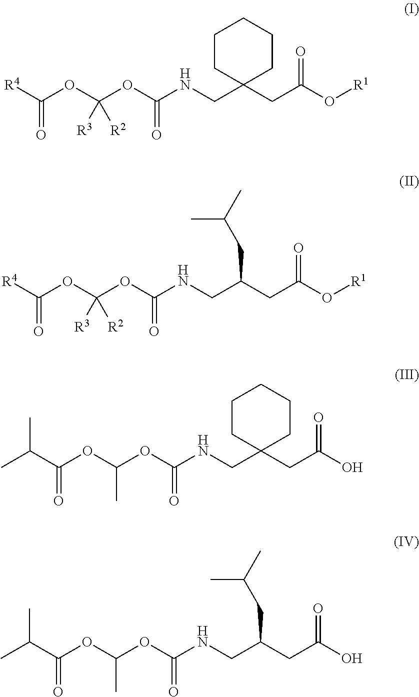
a pharmaceutically acceptable salt of any of the foregoing, a pharmaceutically acceptable solvate of any of the foregoing, and a pharmaceutically acceptable N-oxide of any of the foregoing, wherein:
R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a ring chosen from cycloalkyl, substituted cycloalkyl, cycloheteroalkyl, and substituted cycloheteroalkyl ring; and
R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

2. The method of claim 1, wherein R¹ is hydrogen.

3. The method of claim 1, wherein R² and R³ are independently chosen from hydrogen and C₁₋₆ alkyl.

4. The method of claim 1, wherein at least one of R² and R³ is other than hydrogen.

5. The method of claim 1, wherein R³ is chosen from methyl, ethyl, n-propyl, isopropyl, n-butyl, isobutyl, and sec-butyl, and R² is hydrogen.

6. (canceled)

7. The method of claim 1, wherein R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

8. The method of claim 1, wherein R¹ and R² are each hydrogen, R³ is C₁₋₆ alkyl, and R⁴ is chosen from C₁₋₆ alkyl and C₁₋₆ substituted alkyl.

9. The method of claim 1, wherein R¹ and R² are each hydrogen, R³ is chosen from methyl, ethyl, n-propyl, isopropyl, n-butyl, isobutyl, and sec-butyl, and R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

10. The method of claim 1, wherein the compound is a compound of Formula (III), 1-{[(α-isobutanoyloxyethoxy)carbonyl]aminomethyl}-1-cyclohexane acetic acid, a pharmaceutically acceptable salt thereof, a pharmaceutical acceptable solvate of any of the foregoing, or a pharmaceutically acceptable N-.oxide of any of the foregoing.

11. (canceled)

12. The method of claim 1, wherein the compound is chosen from Formula (I) and Formula (III) and is administered in an amount from about 10 mg-equivalents to about 3600 mg-equivalents of gabapentin per day.

13. (canceled)

14. The method of claim 1, wherein the compound is administered orally.

15. The method of claim 14, comprising administering the compound in a sustained release oral dosage form.

16. The method of claim 15, wherein a therapeutically effective amount of gabapentin or pregabalin is maintained in the plasma of the patient for a period of at least about 4 hours after administrating the compound.

17. (canceled)

18. The method of claim 1, wherein the disease is fibromyalgia.

19. The method of claim 1, wherein the disease is amyotrophic lateral sclerosis.

20. The method of claim 1, wherein the disease is social phobia.

21. The method of claim 1, wherein the disease is Parkinson's disease.

22. The method of claim 1, wherein the disease is cough.

23. The method of claim 1, wherein the disease is asthma.

24. The method of claim 1, wherein the disease is chronic obstructive pulmonary disease.

25. (canceled)
